(19) United States
(12) Patent Application Publication
(10) Pub. No.: US 2011/0113498 A1
(43) Pub. Date: May 12, 2011

(54) SIRT1 POLYMORPHIC VARIANTS AND METHODS OF USE THEREOF

(76) Inventors: Christoph H. Westphal, Cambridge, MA (US); Marc Donath, Zurich (CH); Peter Elliott, Cambridge, MA (US); Michael Jirousek, Cambridge, MA (US); Jill Milne, Cambridge, MA (US)

(21) Appl. No.: 12/992,765

(22) PCT Filed: May 15, 2009
(86) PCT No.: PCT/US2009/044054

§ 371 Date: Nov. 15, 2010

Related U.S. Application Data
(60) Provisional application No. 61/127,716, filed on May 15, 2008.

(51) Int. Cl. C12Q 1/68; C07H 21/00; C12N 15/63; C40B 40/08; C12N 9/78; C12N 1/21; A01K 67/027

(52) U.S. Cl. 800/14; 536/23.2; 435/320.1; 506/17; 435/227; 435/252.33; 435/6

(57) ABSTRACT

Provided herein are Sirt1 polymorphic variants having a substitution at amino acid residue 107 or nucleotide 373. In certain embodiments, the Sirt1 polypeptide variants have a L107P substitution and the nucleic acid variants have a T373C substitution. Genetic and/or biochemical testing may be performed to identify whether a patient carries one of the disclosed polymorphic variants. Based on the polymorphic variant the patient carries, a medical practitioner may administer an appropriate therapy, such as a sirtuin activator.

### BACKGROUND

Type 1 and Type 2 Diabetes Mellitus have become a significant epidemic throughout the world. There is a significant unmet medical need for novel mechanism of action therapeutics for the treatment of metabolic diseases such as diabetes. One novel therapeutic approach to treating insulin resistance and diabetes has come from the study of Calorie Restriction (CR), a dietary regimen of consuming 30-40% fewer calories, which has been shown to improve a number of metabolic parameters including insulin sensitivity (Heilbronn, L. K. & Ravussin, E., Calorie restriction and aging: review of the literature and implications for studies in humans. Am J Clin Nutr 78, 361-9 (2003); Roth, G. S., Ingram, D. K. & Lane, M. A. Caloric restriction in primates and relevance to humans Ann N Y Acad Sci 928, 305-15 (2001)). The molecular components of the pathway(s) downstream of CR may provide relevant intervention points for the development of therapeutic drugs to treat metabolic disease (Weindruch, R. et al. Caloric restriction mimetics: metabolic interventions. J Gerontol A Biol Sci Med Sci 56 Spec No 1, 20-33 (2001); Ingram, D. K. et al. Calorie restriction mimetics: an emerging research field. Aging Cell 5, 97-108 (2006)).

Studies in lower organisms including Saccharomyces cerevisiae and Drosophila melanogaster have led to the identification of key players in these pathways. The protein Sir2 has been identified as one such player that may mediate some of the physiological benefits of CR (Bordone, L. & Guarente, L. Calorie restriction, Sirt1 and metabolism: understanding longevity. Nat Rev Mol Cell Biol 6, 298-305 (2005); Sinclair, D. A. & Guarente, L. Extrachromosomal rDNA circles—a cause of aging in yeast. Cell 91, 1033-42 (1997)). In yeast and flies, Sir2 is a histone deacetylase that when overexpressed extends lifespan, and when deleted decreases lifespan (Kaeberlein, M., McVey, M. & Guarente, L. The SIR2/3/4 complex and SIR2 alone promote longevity in Saccharomyces cerevisiae by two different mechanisms. Genes Dev 13, 2570-80 (1999); Rogina, B. & Helfand, S. L. Sir2 mediates longevity in the fly through a pathway related to calorie restriction. Proc Natl Acad Sci USA 101, 15998-6003 (2004)). In addition, the ability of CR to extend lifespan in yeast and flies is abrogated when Sir2 is deleted underscoring the importance of this protein in pathways downstream of CR (Rogina (2004), supra; Anderson, R. M., Bitterman, K. J., Wood, J. G., Medvedik, O. & Sinclair, D. A. Nicotinamide and PNC1 govern lifespan extension by calorie restriction in Saccharomyces cerevisiae. Nature 423, 181-5 (2003); Lin, S. J., Defossez, P. A. & Guarente, L. Requirement of NAD and SIR2 for life-span extension by calorie restriction in Saccharomyces cerevisiae. Science 289, 2126-8 (2000)).

The Sir2 homolog in mammals is Sirt1. Several lines of data support a link between Sirt1 and CR, and suggest a role for this enzyme in mediating some of the health benefits of CR in mammals. Sirt1 levels in several tissues in rodents are increased following a regimen of CR (Cohen, H. Y. et al. Calorie restriction promotes mammalian cell survival by inducing the Sirt1 deacetylase. Science 305, 390-2 (2004); Heilbronn, L. K. et al. Glucose tolerance and skeletal muscle gene expression in response to alternate day fasting. Obes Res 13, 574-81 (2005)). Resveratrol, a compound that has been shown to induce activation of Sirt1 and mimic the effects of CR in lower organisms, has recently been shown to improve insulin sensitivity, increase mitochondrial content, and survival of mice on a high calorie diet (Howitz, K. T. et al. Small molecule activators of sirtuins extend Saccharomyces cerevisiae lifespan. Nature 425, 191-6 (2003); Jarolim, S. et al. A novel assay for replicative lifespan in Saccharomyces cerevisiae. FEMS yeast research 5, 169-77 (2004); Wood, J. G. et al. Sirtuin activators mimic caloric restriction and delay ageing in metazoans. Nature (London, United Kingdom) 430, 686-689 (2004); Baur, J. A. et al. Resveratrol improves health and survival of mice on a high-calorie diet. Nature (2006)).

Sirt1 is a member of the sirtuin family of NAD⁺-dependent deacetylases. These enzymes have evolved to catalyze a unique reaction in which deacetylation of a lysine residue in a substrate protein is coupled to the consumption of NAD⁺ (Frye, R. A. Characterization of five human cDNAs with homology to the yeast SIR2 gene: Sir2-like proteins (sirtuins) metabolize NAD and may have protein ADP-ribosyltransferase activity. Biochem Biophys Res Commun 260, 273-9 (1999); Frye, R. A. Phylogenetic classification of prokaryotic and eukaryotic Sir2-like proteins. Biochem Biophys Res Commun 273, 793-8 (2000); Imai, S., Armstrong, C. M., Kaeberlein, M. & Guarente, L. Transcriptional silencing and longevity protein Sir2 is an NAD-dependent histone deacetylase. Nature 403, 795-800 (2000)). A number of cellular substrates for Sirt1 have been identified including PGC-1α, NCoR, p300, NFkB, Foxo, and p53 (Bouras, T. et al. Sirt1 deacetylation and repression of p300 involves lysine residues 1020/1024 within the cell cycle regulatory domain 1. J Biol Chem 280, 10264-76 (2005); Brunet, A. et al. Stress-dependent regulation of FOXO transcription factors by the Sirt1 deacetylase. Science 303, 2011-5 (2004); Luo, J. et al. Negative control of p53 by Sir2alpha promotes cell survival under stress. Cell 107, 137-48 (2001); Motta, M. C. et al. Mammalian Sirt1 represses forkhead transcription factors. Cell 116, 551-63 (2004); Nemoto, S., Fergusson, M. M. & Finkel, T. Sirt1 functionally interacts with the metabolic regulator and transcriptional coactivator PGC-1alpha. J Biol Chem 280, 16456-60 (2005); Picard, F. et al. Sirt1 promotes fat mobilization in white adipocytes by repressing PPAR-gamma. Nature 429, 771-6 (2004); Rodgers, J. T. et al. Nutrient control of glucose homeostasis through a complex of PGC-1alpha and Sirt1. Nature 434, 113-8 (2005); van der Horst, A. et al. FOXO4 is acetylated upon peroxide stress and deacetylated by the longevity protein hSir2(Sirt1). J Biol Chem 279, 28873-9 (2004); Vaziri, H. et al. hSIR2(Sirt1) functions as an NAD-dependent p53 deacetylase. Cell 107, 149-59 (2001); Yeung, F. et al. Modulation of NF-kappaB-dependent transcription and cell survival by the Sirt1 deacetylase. Embo J 23, 2369-80 (2004)). Through modulation of the activities of these proteins, Sirt1 regulates mitochondrial biogenesis, metabolism in muscle and adipose tissue, and cellular survival (Cohen (2004) supra; Heilbronn (2005), supra; Picard (2004), supra; Nisoli, E. et al. Calorie restriction promotes mitochondrial biogenesis by inducing the expression of eNOS. Science 310, 314-7 (2005)).

### SUMMARY

Provided herein are Sirt1 polymorphic variants having an amino acid other than leucine at position 107, or a nucleotide other than thymine at position 373. In certain embodiments, amino acid 107 is a proline and nucleotide 373 is a cytosine. Genetic and/or biochemical testing may be performed to identify whether a patient carries one of the disclosed polymorphic variants. Based on the polymorphic variant the patient carries, a medical practitioner may administer an appropriate therapy, such as a sirtuin activator.

In one aspect, the application provides an isolated nucleic acid comprising a Sirt1 polymorphic variant having a nucleotide other than thymine at nucleotide 373. In some embodiments, nucleotide 373 is cytosine. In some embodiments, the isolated nucleic acid has the sequence of SEQ ID NO: 1, wherein N is cytosine, guanine or adenine. In a related aspect, the variants comprise an isolated Sirt1 nucleic acid having a T373N substitution comprising: SEQ ID NO: 1 or a nucleotide sequence having 97% identity to SEQ ID NO: 1, wherein the nucleotide at position 373 is adenine, guanine, or cytosine. The isolated nucleic acid may be operably linked to a nucleotide tag sequence, or a promoter sequence. Furthermore, the instant disclosure provides a vector comprising any of the above nucleic acids.

This disclosure also comprises an isolated oligonucleotide comprising, for example, 8 to 20, 10 to 15, 10 to 50, 20 to 50, 50 to 70, 30 to 80, 20 to 100, 20 to 200, or 20 to 300 consecutive nucleotides, from SEQ ID No. 1 or the complement thereof, wherein nucleotide 373 is included in said oligonucleotide. This isolated oligonucleotide may be attached to a solid substrate, for example as part of a microarray or as part of a nucleic acid hybridization assay. The isolated oligonucleotide may be labeled with a detectable label. The present application also discloses a microarray comprising a solid substrate and a plurality of oligonucleotides, wherein at least one oligonucleotide is an oligonucleotide disclosed herein.

Also provided, is an isolated polypeptide comprising a Sirt1 polymorphic variant, wherein amino acid 107 is an amino acid other than leucine. Amino acid 107 may be proline. In some aspects, the isolated polypeptide has the sequence of SEQ ID NO: 2, wherein X is an amino acid other than leucine. In a related aspect, the polypeptides comprise an isolated Sirt1 polypeptide having a L107X substitution comprising: SEQ ID NO: 2 or an amino acid sequence having 99% identity to SEQ ID NO: 2, wherein the amino acid at position 107 is any amino acid other than leucine. The isolated polypeptide may be operably linked to a polypeptide tag sequence. This disclosure also contemplates a nucleic acid encoding the isolated polypeptides taught herein, as well as a host cell comprising any nucleic acid, polypeptide, oligonucleotide, or vector taught herein. The host cell may be a bacterial host cell or a mammalian cell (notably, a human cell or a murine cell). Additionally, Applicants provide a non-human transgenic mammal (such as a mouse) comprising a nucleic acid, polypeptide, oligonucleotide, or vector as set out in this application.

In another aspect, the application provides an antibody or antigen-binding portion thereof having an affinity for a polypeptide having a L107X substitution that is at least 2, 5, 10, 20, 50, or 100 times the affinity of said antibody or antigen-binding portion thereof for a Sirt1 polypeptide having a leucine at position 107. The antibody or antigen-binding portion thereof may be attached to a solid substrate. Applicants also provide a protein microarray comprising a solid substrate and a plurality of antibodies or antigen-binding portions thereof, wherein at least one antibody or antigen-binding portion thereof is an antibody or antigen-binding portion thereof disclosed herein.

Applicants also provide a method for evaluating a subject's risk of developing a sirtuin mediated disease or disorder, comprising determining the identity of nucleotide 373 or amino acid 107 of Sirt1 in a biological sample from said subject. In this method, a nucleotide other than thymine at position 373 or an amino acid other than leucine at position 107 may be indicative of a subject at risk for developing a sirtuin mediated disease or disorder.

Also provided is a method for identifying a subject that would be responsive to or would benefit from treatment with a sirtuin modulating compound, comprising determining the identity of nucleotide 373 or amino acid 107 of Sirt1 in a biological sample from said subject. In this method, a nucleotide other than thymine at position 373 or an amino acid other than leucine at position 107 may be indicative of a subject that would be responsive to or would benefit from treatment with a sirtuin modulating compound. The sirtuin-mediated disease or disorder may be, for example, an autoimmune disease (such as ulcerative colitis) or a metabolic disease such as diabetes (such as type 1 and type 2 diabetes). In these methods, the identity of nucleotide 373 can be determined by nucleic acid sequencing, primer extension, restriction enzyme cleavage pattern, or by use of a nucleic acid probe that hybridizes to the nucleic acid sequence. Alternatively, the identity of amino acid 107 can be determined by mass spectrometry or binding of an antibody or antigen-binding portion of said antibody. In certain aspects, a cytosine at nucleotide 373 is indicative of a subject at risk for developing a sirtuin mediated disease or disorder. In certain embodiments, a cytosine at nucleotide 373 is indicative of a subject that would be responsive to or would benefit from treatment with a sirtuin modulating compound. In other embodiments, a proline at amino acid 107 is indicative of a subject at risk for developing a sirtuin mediated disease or disorder. Additionally, a proline at amino acid 107 may be indicative of a subject that would be responsive to or would benefit from treatment with a sirtuin modulating compound.

The methods herein may further comprise performing at least one additional test to measure the risk of developing a sirtuin mediated disease, such as a metabolic disease, e.g. diabetes. Such a test for diabetes may comprise determining the nucleotide or amino acid sequence of one, more than one, part of one, or part of more than one of the following genes: DQ HLA allele, L-selectin, PPAR gamma, hepatocyte nuclear factor 1-a, HNF4-a, Insulin receptor substrate-1, Insulin receptor substrate-2, PGC-1 alpha, KCNJI1, ABCC8, GLUT1, GLUT4, calpain 10, glucagon receptor, human beta 3 adrenergic receptor, fatty acid binding protein 2, mitochondrial tRNA [Leu (UUR)], sulphonylurea receptor, UCP2, UCP3, PTPN1, adiponectin, TCF7L2, and amylin, and the regulatory nucleotide sequences thereof.

Alternatively, the diabetes test may comprise a fasting glucose test, glucose tolerance test, lipid profile test, sdLDL test, fasting insulin test, microalbumin test, hs-CRP test, blood pressure test, or test for obesity. The biological sample may be a blood sample, serum sample, or tissue sample. In these methods, the at least one additional test may be a test for an autoimmune disease such as a titer test for autoantibodies. Autoantibodies can be detected in serum, or if the symptoms are localized, in a biopsy of the affected tissue, for example by immunofluorescence or immunohistochemistry. Other tests for autoimmune diseases include tests for elevated levels of C-reactive protein or certain cytokines.

In another aspect, the application provides a method for treating a sirtuin mediated disease or disorder in a subject, comprising: a) determining the identity of nucleotide 373 or amino acid 107 of Sirt1 in a biological sample from said subject; b) analyzing the identity of nucleotide 373 or amino acid 107 of Sirt1 to determine a course of treatment, dosage regimen, or course of treatment and dosage regimen for said subject; and c) administering a sirtuin-modulating compound to said subject according to the determined course of treatment, dosage regimen, or course of treatment and dosage regimen, thereby treating the sirtuin mediated disease or disorder. In some embodiments, the sirtuin-modulating compound is a sirtuin-activating compound such as resveratrol, fisetin, butein, piceatannol or quercetin. In some embodiments, the sirtuin mediated disease is a metabolic disease or an autoimmune disease. In different embodiments, the sirtuin modulating compound modulates the activity of Sirt1, Sirt2, Sirt3, Sirt4, Sirt5, Sirt6, and/or Sirt7.

The present application also teaches a method of determining the activity of a Sirt1 polymorphic variant polypeptide, comprising: a) contacting a peptide substrate pool with the Sirt1 polymorphic variant polypeptide, wherein members of said peptide substrate pool comprise at least one acetylated lysine residue, and b) determining the acetylation level of the peptide substrate pool, wherein the Sirt1 polymorphic variant polypeptide has an amino acid other than leucine at position 107. This method may further comprise: contacting the peptide substrate pool with a reagent that cleaves members of the peptide substrate pool having non-acetylated lysine residues; and determining the fluorescence polarization value of the peptide substrate pool, wherein a decrease in the fluorescence polarization value of the peptide substrate pool is indicative of an increase in Sirt1 polymorphic variant polypeptide activity, wherein members of said peptide substrate pool comprise a fluorophore. The method may further comprise contacting the Sirt1 polymorphic variant polypeptide with a test agent and measuring Sirt1 activity after contact with said test agent. Fluorescence polarization assays are known in the art, for example see WO 2006/094239.

In another aspect, the application provides a method for identifying a compound that modulates a Sirt1 polymorphic variant polypeptide, comprising contacting a peptide substrate pool with the Sirt1 polymorphic variant polypeptide in the presence of a test compound, wherein members of said peptide substrate pool comprise at least one acetylated lysine residue, and determining the acetylation level of the peptide substrate pool, wherein the Sirt1 polymorphic variant polypeptide has an amino acid other than leucine at position 107. This method may further comprise: contacting the peptide substrate pool with a reagent that cleaves members of the peptide substrate pool having non-acetylated lysine residues; and determining the fluorescence polarization value of the peptide substrate pool, wherein a change in the fluorescence polarization value of the peptide substrate pool in the presence of the test compound as compared to a control is indicative of a compound that modulates Sirt1 polymorphic variant activity, wherein members of said peptide substrate pool comprise a fluorophore. In certain embodiments, a compound that increases the activity of the Sirt1 polymorphic variant polypeptide is identified. In different embodiments, the peptide substrate pool is in vitro, in a cell, or in an organism. In certain embodiments, amino acid 107 is a proline.

Also provided herein is a method for evaluating a Sirt1-modulating compound, comprising: a) administering a Sirt1-modulating compound to a patient population; b) determining the identity of nucleotide 373 or amino acid 107 of Sirt1 in a biological sample from the patients in said population before or after administering said Sirt1-modulating compound to said patient population; c) evaluating the efficacy of the Sirt1-modulating compound in said patient population; and d) correlating the efficacy of the Sirt1-modulating compound with identity of nucleotide 373 or amino acid 107 of Sirt1, thereby evaluating the Sirt1 variant-modulating compound. Also disclosed is a method for evaluating a Sirt1-modulating compound, comprising: a) administering a Sirt1-modulating compound to a patient population for which the identity of nucleotide 373 or amino acid 107 of Sirt1 has been determined; b) evaluating the efficacy of the Sirt1-modulating compound in said patient population; and c) correlating the efficacy of the Sirt1-modulating compound with the identity of nucleotide 373 or amino acid 107 of Sirt1, thereby evaluating the Sirt1-modulating compound. In certain embodiments, the Sirt1-modulating compound is a Sirt1-activating compound.

In another aspect, the application provides a method for evaluating a sirtuin modulating compound, comprising: a) administering a sirtuin modulating compound to a patient population; b) determining the identity of nucleotide 373 or amino acid 107 of Sirt1 in a biological sample from the patients in said population before or after administering said sirtuin modulating compound to said patient population; c) evaluating the efficacy of the sirtuin modulating compound in said patient population; and d) correlating the efficacy of the sirtuin modulating compound with identity of nucleotide 373 or amino acid 107 of Sirt1, thereby evaluating the sirtuin modulating compound.

In another aspect, the application provides a method for evaluating a sirtuin modulating compound, comprising: a) administering a sirtuin modulating compound to a patient population for which the identity of nucleotide 373 or amino acid 107 of Sirt1 has been determined; b) evaluating the efficacy of the sirtuin modulating compound in said patient population; and c) correlating the efficacy of the sirtuin modulating compound with the identity of nucleotide 373 or amino acid 107 of Sirt1, thereby evaluating the sirtuin modulating compound. In certain aspects, the sirtuin modulating compound is a sirtuin activating compound. The method, in certain embodiments, calls for the sirtuin modulating compound to modulate one or more of Sirt1, Sirt2, Sirt3, Sirt4, Sirt5, Sirt6, or Sirt7. In certain aspects, the identity of nucleotide 373 is cytosine or thymine and the identity of amino acid 107 is leucine or proline.

In another aspect, the application provides a method for quantifying the predictive value of a mutation at nucleotide 373 or amino acid 107 of Sirt1, comprising: a) determining the identity of nucleotide 373 or amino acid 107 of Sirt1 in a biological sample from patients in a patient population; b) assaying one or more physiological or metabolic parameters in the patients of said patient population; c) correlating the identity of nucleotide 373 or amino acid 107 of Sirt1 with the one or more physiological or metabolic parameters in said patient population, wherein the correlation value is a quantification of the predictive value of the mutation at nucleotide 373 or amino acid 107 of Sirt1. In some embodiments, the physiological or metabolic parameters are measured over time. In certain embodiment, the physiological or metabolic parameters are one or more of the following: energy expenditure, exercise endurance, blood glucose levels, glucose tolerance, insulin responsiveness, or insulin levels.

The appended claims are incorporated into this section by reference.

### BRIEF DESCRIPTION OF THE FIGURES

The foregoing and other features and advantages of the present invention will be more fully understood from the following detailed description of illustrative embodiments taken in conjunction with the accompanying drawings in which:

FIG. 1 portrays family tree of a patient having a Sirt1 mutation. Black symbols indicate family members in whom diabetes developed. Numbered symbols indicate age of onset of diabetes. The gray symbols a patient in whom Colitis developed. Symbols with a slash denote deceased family members.

FIGS. 2A and 2B illustrate plasma glucose levels and plasma insulin levels in a subject carrying a Sirt1 mutation and a control subject with wild-type Sirt1 during an oral glucose tolerance tests. The subject carrying the Sirt1 mutation is the index patient (squares) and the control subject is a 20-year-old non-affected male family member (triangles). Panel A, plasma glucose levels: The y axis indicates glucose levels (in mM) and the x axis indicates time after administration of a bolus of glucose. Panel B, plasma insulin levels: The y axis indicates insulin levels (in pM) and the x axis indicates time after administration of a bolus of glucose.

FIG. 3 shows the data obtained by sequencing the Sirt1 gene in different subjects. Upper left panel, raw sequencing data obtained by sequencing the Sirt1 gene of a subject having a Sirt1 mutation. Upper right panel, DNA (SEQ ID NO: 43) and protein sequence (SEQ ID NO: 44) of mutant Sirt1 (L107P) extrapolated from the sequencing of mutant Sirt1. Lower left panel, raw sequencing data obtained by sequencing the Sirt1 gene of a subject having a wild-type copy of Sirt1. Lower right panel, DNA (SEQ ID NO: 45) and protein sequence (SEQ ID NO: 46) of mutant Sirt1 extrapolated from the sequencing of wild-type Sirt1.

FIG. 4 shows the human Sirt1 nucleic acid sequence with residue 373 indicated as an N (SEQ ID NO: 1) and N represents any nucleotide. In wild-type Sirt1, residue 373 is a thymine. Variant Sirt1 nucleic acids have a T373N mutation wherein N is A, G or C. In an exemplary embodiment, a variant Sirt1 nucleic acid has a T373C mutation.

FIG. 5 shows the human Sirt1 protein sequence with residue 107 indicated as an X (SEQ ID NO: 2) and X represents any amino acid. In wild-type Sirt1, residue 107 is a leucine. Variant Sirt1 proteins have a L107X mutation wherein L is leucine and X is any residue other than leucine. In an exemplary embodiment, a variant Sirt1 protein has a L107P mutation where L is leucine and P is proline.

### DETAILED DESCRIPTION

Studies provided herein have linked metabolic diseases (such as diabetes) and autoimmune diseases (such as ulcerative colitis) with the T373C and L107P polymorphic variants of Sirt1. Sirtuin activity has been linked to a variety of diseases and disorders including, for example, diseases or disorders related to aging or stress, diabetes, cancer, obesity, neurodegenerative diseases, diseases or disorders associated with mitochondrial dysfunction, chemotherapeutic induced neuropathy, neuropathy associated with an ischemic event, ocular diseases and/or disorders, cardiovascular disease, blood clotting disorders, inflammation, and/or flushing, etc.

1. Sirt1 Polymorphic Variants Associated with Sirtuin Mediated Diseases and Disorders

Provided herein are T373N and L107X Sirt1 polymorphic variants that are associated with a sirtuin mediated diseases or disorders.

The terminology for nucleotide and amino acid substitutions used herein is as follows. For Sirt1 polypeptide variants, the first letter represents the amino acid residue (represented using the one letter code) that is naturally present in the human Sirt1 polypeptide sequence. The following number represents the position of the amino acid residue in the full length human Sirt1 amino acid sequence (SEQ ID NO: 2). The second letter represents the amino acid substituting for or replacing the naturally occurring amino acid at the specified position. As an example, L107P denotes that the leucine residue at position 107 of the human Sirt1 protein has been replaced with a proline residue. For Sirt1 nucleic acid variants, the first letter represents the nucleotide residue that is naturally present at a position in the human Sirt1 nucleotide or polypeptide sequence. The following number represents the position of the nucleotide residue in the full length human Sirt1 nucleotide sequence (SEQ ID NO: 1). The second letter represents the nucleotide substituting for or replacing the naturally occurring nucleotide at the specified position. As an example, T373C denotes that the thymine residue at position 373 of human Sirt1 nucleic acid sequence has been replaced with a cytosine residue.

In one embodiment, a T373N Sirt1 polymorphic variant is provided. A T373N polymorphic variant refers to a Sirt1 nucleic acid variant having a substitution at position 373 of the human Sirt1 nucleic acid sequence in which the naturally occurring thymine nucleotide has been replaced with a residue other than thymine (T), e.g., a cytosine (C), guanine (G), or adenine (A) nucleotide. A T373N Sirt1 polymorphic variant also includes a Sirt1 polypeptide encoded by a Sirt1 T373N polymorphic variant. An exemplary T373N Sirt1 polymorphic variant is a T373C variant.

In another embodiment, a L107X Sirt1 polymorphic variant is provided. A L107X polymorphic variant refers to a Sirt1 polypeptide variant having a substitution at position 107 of the human Sirt1 amino acid sequence in which the naturally occurring leucine residue has been replaced with an amino acid other than Leucine. A L107X Sirt1 polymorphic variant also includes nucleic acids encoding a Sirt1 L107X polymorphic variant. An exemplary L107X Sirt1 polymorphic variant is a L107P variant.

Isolated Sirt1 nucleic acids comprising a T373N mutation, wherein N is adenine, cytosine or guanine are provided herein. In an exemplary embodiment, N is cytosine. A “Sirt1 nucleic acid” as used herein refers to a nucleic acid encoding full length human Sirt1 polypeptide, or a fragment, analog or derivative thereof. Wild-type Sirt1 nucleic acid refers to SEQ ID NO: 1 wherein N is thymine. An isolated Sirt1 nucleic acid may comprise SEQ ID NO: 1 wherein N is adenine, guanine or cytosine. In other embodiments, a Sirt1 nucleic acid refers to a nucleic acid that is at least at least 70%, 80%, 90%, 95%, 97%, 98%, or 99% identical to SEQ ID NO: 1, wherein N is adenine, guanine, or cytosine. In certain embodiments, a Sirt1 nucleic acid encodes a polypeptide having at least one biological activity of a wild-type Sirt1 polypeptide, such as, deacetylase activity. Isolated Sirt1 nucleic acids also include any nucleic acid encoding a L107X Sirt1 polymorphic variant.

Also provided are isolated Sirt1 polypeptides comprising a L107X mutation, wherein X is any amino acid other then a leucine. In an exemplary embodiment, X is proline. A “Sirt1 polypeptide” as used herein refers to full length human Sirt1 polypeptide, or a fragment, analog or derivative thereof. Wild-type Sirt1 polypeptide refers to SEQ ID NO: 2 wherein X is leucine. An isolated Sirt1 polypeptide may comprise SEQ ID NO: 2 wherein X is an amino acid other than leucine. In other embodiments, a Sirt1 polypeptide refers to a polypeptide comprising an amino acid sequence that is at least at least 70%, 80%, 90%, 95%, 97%, 98%, or 99% identical to SEQ ID NO: 2, wherein X is an amino acid residue other than leucine. In certain embodiments, a Sirt1 polypeptide has at least one biological activity of a wild-type Sirt1 polypeptide, such as, deacetylase activity.

As used herein, “amino acid 107” (or “amino acid at position 107”) refers to the amino acid residue at position 107 of SEQ ID NO: 2. That is, amino acid 107 refers to the amino acid that is flanked by DNGPG in the N-terminal direction and QGPSR in the C-terminal direction, in wild-type Sirt1. The identity of amino acid 107 is not to be offset if, for example, an amino acid sequence is added to the N-terminus of Sirt1. Similarly, “nucleotide 373” (or “nucleotide at position 373”) refers to the nucleotide at position 373 of SEQ ID NO: 1. That is, nucleotide 373 refers to the nucleotide that is flanked by GGGCC upstream of nucleotide 373 and GCAGG downstream of nucleotide 373, in wild-type Sirt1. The identity of nucleotide 373 is not to be offset if, for example, a nucleic acid sequence is added upstream of the first codon of Sirt1. It will be understood that reference to nucleotide 373 and amino acid 107 is made with respect to the human Sirt1 sequence. One of skill in the art will be able to determine the equivalent position in a Sirt1 variant or homolog using standard alignment methods (e.g., using the Blast tool available from NCBI on the world wide web at blast.ncbi.nlm.nih.gov/Blast.cgi). For example, human Sirt1 may be aligned with the mouse Sirt1 homolog to identify the nucleotide or amino acid residue in mouse Sirt1 that corresponds to position 373 of the human Sirt1 nucleic acid or position 107 of the human Sirt1 polypeptide.

In some embodiments, the isolated Sirt1 nucleic acids provided herein may be operably linked to a nucleotide tag sequence, or a promoter sequence. As used herein, a “nucleotide tag sequence” refers to a nucleotide sequence that, when operably linked to a second nucleotide sequence, facilitates identification, isolation, or other manipulation of the second nucleotide sequence or the protein product encoded therein. For example, a nucleotide tag sequence may encode a detectable label such as a fluorescent protein, an affinity purification moiety, or a targeting moiety (such as a nuclear localization sequence or an export sequence). Furthermore, the instant disclosure provides vectors comprising any of the above nucleic acids.

The isolated Sirt1 polypeptides may be operably linked to a polypeptide tag sequence. As used herein, a “polypeptide tag sequence” refers to a polypeptide sequence that, when operably linked to a second polypeptide sequence, facilitates identification, isolation, or other manipulation of the second polypeptide sequence. For example, a polypeptide tag sequence may comprise a detectable label (such as a fluorescent protein or moiety with affinity for a detectable label e.g. FlAsH), an affinity purification moiety, or a targeting moiety (such as a nuclear localization sequence or an export sequence).

“Operably linked” refers to a juxtaposition wherein the components so described are in a relationship permitting them to function in their intended manner. For instance, a promoter is operably linked to a coding sequence if the promoter affects its transcription or expression. In addition, a polypeptide tag operably linked to a protein may direct the localization of that protein to a specific region of a cell, facilitate affinity purification of that protein, and the like.

This disclosure also contemplates host cells comprising any nucleic acid, polypeptide, oligonucleotide, or vector taught herein. The host cell may be a bacterial host cell or a mammalian cell (notably, a human cell or a murine cell). Additionally, Applicants provide a transgenic non-human mammal (such as a mouse) comprising a nucleic acid, polypeptide, oligonucleotide, or vector as set out in this application.

Also provided are isolated oligonucleotides comprising, for example, 8 to 20, 10 to 15, 10 to 50, 20 to 50, 50 to 70, 30 to 80, 20 to 100, 20 to 200, or 20 to 300 consecutive nucleotides, from SEQ ID NO: 1 or the complement thereof, wherein nucleotide 373 is included in said oligonucleotide. The isolated oligonucleotides may be labeled with a detectable label. Exemplary detectable labels include, for example, radioactive isotopes (such as P³² or H³), fluorophores (such as fluorescein isothiocyanate (FITC), TRITC, rhodamine, tetramethylrhodamine, R-phycoerythrin, Cy-3, Cy-5, Cy-7, Texas Red, Phar-Red, or allophycocyanin (APC)), epitope tags (such as the FLAG or HA epitope), enzyme tags (such as alkaline phosphatase, horseradish peroxidase, or beta-galactosidase), hapten conjugates (such as digoxigenin or dinitrophenyl), chemiluminescent molecules, chromogenic molecules, optical or electron density markers, or semiconductor nanocrystals such as quantum dots (i.e., Qdots) (see e.g., U.S. Pat. No. 6,207,392). The isolated oligonucleotides may be attached to a solid substrate, for example as part of a microarray or as part of a nucleic acid hybridization assay. The present application also provides a microarray comprising a solid substrate and a plurality of oligonucleotides, wherein at least one oligonucleotide is an oligonucleotide disclosed herein.

In another embodiment, the application provides an antibody or antigen-binding portion thereof having a binding affinity for L107X Sirt1 variant that is at least two-fold greater than the binding affinity of the antibody or antigen-binding portion thereof for wild-type Sirt1. In certain embodiments, the antibody or antigen-binding portion thereof has a binding affinity for L107X Sirt1 variant that is at least five-fold, ten-fold, twenty-fold, fifty-fold, or 100-fold greater than the binding affinity of the antibody or antigen-binding portion thereof for wild-type Sirt1. The antibody or antigen-binding portion thereof may be attached to a solid substrate. Applicants also provide a protein microarray comprising a solid substrate and a plurality of antibodies or antigen-binding portions thereof, wherein at least one antibody or antigen-binding portion thereof is an antibody or antigen-binding portion thereof disclosed herein.

As used herein, the term “polymorphic variant” refers to an allele of a gene, wherein at least two different alleles of the same gene are observed in a population, or a gene product thereof. Compared to one polymorphic variant, another polymorphic variant may have a substitution mutation, an inserted nucleotide or nucleotide sequence, a deleted nucleotide or nucleotide sequence, or a microsatellite, for example. One polymorphic variant often differs from another polymorphic variant of the same gene by a single nucleotide, which varying nucleotide is referred to herein as a “single nucleotide polymorphism” or a “SNP.”

Where two polymorphic variants exist, for example, the polymorphic variant represented in a minority of samples from a population is sometimes referred to as a “minor allele” and the polymorphic variant that is more prevalently represented is sometimes referred to as a “major allele.” In certain embodiments, the major allele of Sirt1 has leucine at amino acid 107, and the minor allele has proline at amino acid 107. Many organisms possess a copy of each chromosome (e.g., humans), and those individuals who possess two major alleles or two minor alleles are often referred to as being “homozygous” with respect to the polymorphism, and those individuals who possess one major allele and one minor allele are normally referred to as being “heterozygous” with respect to the polymorphism. Individuals who are homozygous with respect to one allele are sometimes predisposed to a different phenotype as compared to individuals who are heterozygous or homozygous with respect to another allele.

In the genetic studies presented herein that associate metabolic diseases (such as diabetes) and autoimmune diseases (such as ulcerative colitis) with polymorphic variants of Sirt1, samples from healthy, normal weight, non-diabetic and diabetic patients were allelotyped and genotyped. The term “genotyped” as used herein refers to a process for determining a genotype of one or more individuals, where a “genotype” is a representation of one or more polymorphic variants in a population.

As used herein, the term “phenotype” refers to a trait which can be compared between individuals, such as presence or absence of a condition, a visually observable difference in appearance between individuals, metabolic variations, physiological variations, variations in the function of biological molecules, and the like. An example of a phenotype is occurrence of diabetes.

Methods for detecting a polymorphic variant in a population are described herein. A polymorphic variant may be detected on either or both strands of a double-stranded nucleic acid. For example, a thymine at a particular position in a given sequence can be reported as an adenine from the complementary strand.

### 2. Methods for Detecting Polymorphic Variants

In certain embodiments, the methods described herein involve determining the presence or absence of a T373N or L107X Sirt1 polymorphic variant. Any method for determining the presence or absence of a polymorphic variant may be used in accordance with the methods described herein. Such methods include, for example, detection of a polymorphic variant in a nucleic acid sequence such as genomic DNA, cDNA, or mRNA. T373N or L107X Sirt1 polymorphic variants are located in the coding region or exon region of Sirt1. T373N or L107X Sirt1 polymorphic variants may be associated with differences in gene expression (mRNA and/or protein), post-transcriptional regulation and/or protein activity. For such polymorphic variants, determining the presence or absence of the polymorphic variant may involve determining the level of transcription, mRNA maturation, splicing, translation, protein level, protein stability, and/or protein activity. T373N or L107X Sirt1 polymorphic variants that lead to a change in protein sequence may also be determined by identifying a change in protein sequence and/or structure. A variety of methods for detecting and identifying polymorphic variants are known in the art and are described herein.

Polymorphic variants may be detected in a subject using a biological sample from said patient. Various types of biological samples may be used to detect the presence or absence of a polymorphic variant in said subject, such as, for example, samples of blood, serum, urine, saliva, cells (including cell lysates), tissue, hair, etc. Biological samples suitable for use in accordance with the methods described herein will comprise a Sirt1 nucleic acid or polypeptide sequence. Biological samples may be obtained using known techniques such as venipuncture to obtain blood samples or biopsies to obtain cell or tissue samples.

Diagnostic procedures may also be performed in situ directly upon tissue sections (fixed and/or frozen) obtained from a patient such that no nucleic acid purification is necessary. Nucleic acids may be used as probes and/or primers for such in situ procedures (see, for example, Nuovo, G. J., 1992, PCR in situ hybridization: protocols and applications, Raven Press, New York).

The methods described herein may be used to determine the genotype of a subject with respect to both copies of the polymorphic site present in the genome. For example, the complete genotype may be characterized as −/−, as −/+, or as +/+, where a minus sign indicates the presence of the reference sequence at the polymorphic site, and the plus sign indicates the presence of a polymorphic variant other than the reference sequence. If multiple polymorphic variants exist at a site, this can be appropriately indicated by specifying which ones are present in the subject. Any of the detection means described herein may be used to determine the genotype of a subject with respect to one or both copies of the polymorphism present in the subject's genome.

According to certain embodiments of the invention it is preferable to employ methods that can detect the presence of multiple polymorphic variants (e.g., polymorphic variants at a plurality of polymorphic sites) in parallel or substantially simultaneously. Oligonucleotide arrays represent one suitable means for doing so. Other methods, including methods in which reactions (e.g., amplification, hybridization) are performed in individual vessels, e.g., within individual wells of a multi-well plate or other vessel may also be performed so as to detect the presence of multiple polymorphic variants (e.g., polymorphic variants at a plurality of polymorphic sites) in parallel or substantially simultaneously according to certain embodiments of the invention.

Examples of techniques for detecting differences of at least one nucleotide between two nucleic acids include, but are not limited to, selective oligonucleotide hybridization, selective amplification, or selective primer extension.

A preferred detection method is allele specific hybridization using probes overlapping the polymorphic site and having about 5, 10, 20, 25, or 30 nucleotides around the polymorphic site. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al (1989) Proc. Natl. Acad. Sci. USA 86:6230; and Wallace et al. (1979) Nucl. Acids Res. 6:3543). Such allele specific oligonucleotide hybridization techniques may be used for the simultaneous detection of several nucleotide changes in different polymorphic regions of gene. Examples of probes for detecting specific polymorphic variants of the Sirt1 gene are probes comprising about 5, 10, 20, 25, 30, 50, 75 or 100 nucleotides of SEQ ID NO: 1 or about 5, 10, 20, 25, 30, 50, 75 or 100 nucleotides of a sequence complementary to SEQ ID NO: 1, wherein said probes include nucleotide 373. In one embodiment, oligonucleotides having nucleotide sequences of specific polymorphic variants are attached to a hybridizing membrane and this membrane is then hybridized with labeled sample nucleic acid. Analysis of the hybridization signal will then reveal the identity of the polymorphic variants of the sample nucleic acid. In a preferred embodiment, several probes capable of hybridizing specifically to polymorphic variants are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described e.g., in Cronin et al. (1996) Human Mutation 7:244 and in Kozal et al. (1996) Nature Medicine 2:753. The solid phase support may be contacted with a test nucleic acid and hybridization to the specific probes may be detected. Accordingly, the identity of numerous polymorphic variants of one or more genes, including Sirt1, can be identified in a simple hybridization experiment. For example, the identity of the T373N or L107X Sirt1 polymorphic variant can be determined in a single hybridization experiment, optionally in conjunction with detection of other polymorphic variants of Sirt1 or polymorphic variants of other genes. For example, the identity of the T373N or L107X Sirt1 polymorphic variant can be determined in conjunction with other Sirt1 polymorphic variants such as one or more of the following: SNPs rs12778366, rs3740051, rs2236319, rs2273773, and rs10997870 (see PCT/US2007/022982), in a single hybridization experiment. Alternatively, the identity of the T373N or L107X Sirt1 polymorphic variant can be determined in conjunction with determining the nucleotide or amino acid sequence of a DQ HLA allele, L-selectin, PPAR gamma, hepatocyte nuclear factor 1-a, HNF4-a, Insulin receptor substrate-1, Insulin receptor substrate-2, PGC-1 alpha, KCNJI1, ABCC8, GLUT1, GLUT4, calpain 10, glucagon receptor, human beta 3 adrenergic receptor, fatty acid binding protein 2, mitochondrial tRNA [Leu (UUR)], sulphonylurea receptor, UCP2, UCP3, PTPN1, adiponectin, TCF7L2, or amylin, or the regulatory nucleotide sequences thereof.

Alternatively, allele specific amplification technology which depends on selective PCR amplification may be used. Oligonucleotides used as primers for specific amplification may carry the polymorphic variant of interest in the center of the molecule (so that amplification depends on differential hybridization) (Gibbs et al. (1989) Nucleic Acids Res. 17:2437-2448) or at the extreme 3′ end of one primer where, under appropriate conditions, a mismatch can prevent or reduce polymerase extension (Prossner (1993) Tibtech 11:238; Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al. (1992) Mol. Cell. Probes 6:1).

Various detection methods described herein involve first amplifying at least a portion of a gene prior to identifying the polymorphic variant. Amplification can be performed, e.g., by PCR and/or LCR, according to methods known in the art. In one embodiment, genomic DNA of a cell is exposed to two PCR primers and amplification is carried out for a number of cycles that is sufficient to produce the required amount of amplified DNA. The primers may be about 5-50, about 10-50, about 10-40, about 10-30, about 10-25, about 15-50, about 15-40, about 15-30, about 15-25, or about 25-50 nucleotides in length and may be designed to hybridize to sites about 40-500 base pairs apart (e.g., to amplify a nucleotide sequence of about 40-500 base pairs in length). Exemplary primers for amplifying a region of Sirt1 comprising nucleotide 373 are provided in the examples.

Additional amplification methods include, for example, self sustained sequence replication (Guatelli, J. C. et al., 1990, Proc. Natl. Acad. Sci. U.S.A. 87:1874-1878), transcriptional amplification system (Kwoh, D. Y. et al., 1989, Proc. Natl. Acad. Sci. U.S.A. 86:1173-1177), Q-Beta Replicase (Lizardi, P. M. et al., 1988, Bio/Technology 6:1197), or any other nucleic acid amplification method, followed by the detection of the amplified molecules using techniques well known to those of skill in the art. These detection schemes are especially useful for the detection of nucleic acid molecules that may be present in very low numbers.

Any of a variety of sequencing reactions known in the art can be used to directly sequence at least a portion of a gene and detect polymorphic variants by comparing the sequence of the sample sequence with the corresponding control sequence. Exemplary sequencing reactions include those based on techniques developed by Maxam and Gilbert (Proc. Natl. Acad Sci USA (1977) 74:560) or Sanger (Sanger et al (1977) Proc. Nat. Acad. Sci 74:5463). It is also contemplated that any of a variety of automated sequencing procedures may be utilized to identify polymorphic variants (Biotechniques (1995) 19:448), including sequencing by mass spectrometry. See, for example, U.S. Pat. No. 5,547,835 and international patent application Publication Number WO 94/16101, entitled DNA Sequencing by Mass Spectrometry by H. Koster; U.S. Pat. No. 5,547,835 and international patent application Publication Number WO 94121822 entitled “DNA Sequencing by Mass Spectrometry Via Exonuclease Degradation” by H. Koster, and U.S. Pat. No. 5,605,798 and International Patent Application No. PCT/US96/03651 entitled DNA Diagnostics Based on Mass Spectrometry by H. Koster; Cohen et al. (1996) Adv Chromatogr 36:127-162; and Griffin et al. (1993) Appl Biochem Biotechnol 38:147-159. It will be evident to one skilled in the art that, for certain embodiments, the occurrence of only one, two or three of the nucleic acid bases need be determined in the sequencing reaction. For instance, for a single nucleotide run, such as an A-track, only one nucleotide needs to be detected and therefore modified sequencing reactions can be carried out.

Yet other suitable sequencing methods are disclosed, for example, in U.S. Pat. No. 5,580,732 entitled “Method of DNA sequencing employing a mixed DNA-polymer chain probe” and U.S. Pat. No. 5,571,676 entitled “Method for mismatch-directed in vitro DNA sequencing”.

In some cases, the presence of a specific polymorphic variant in a DNA sample from a subject can be shown by restriction enzyme analysis. For example, a specific polymorphic variant can result in a nucleotide sequence comprising a restriction site which is absent from a nucleotide sequence of another polymorphic variant.

In other embodiments, alterations in electrophoretic mobility may be used to identify the polymorphic variant. For example, single strand conformation polymorphism (SSCP) may be used to detect differences in electrophoretic mobility between polymorphic variants (Orita et al. (1989) Proc Natl. Acad. Sci. USA 86:2766, see also Cotton (1993) Mutat Res 285:125-144; and Hayashi (1992) Genet Anal Tech Appl 9:73-79). Single-stranded DNA fragments of sample and control nucleic acids are denatured and allowed to renature. The secondary structure of single-stranded nucleic acids varies according to sequence and the resulting alteration in electrophoretic mobility enables the detection of even a single base change. The DNA fragments may be labeled or detected with labeled probes. The sensitivity of the assay may be enhanced using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (see e.g., Keen et al. (1991) Trends Genet. 7:5).

In yet another embodiment, the identity of a Sirt1 polymorphic variant may be obtained by analyzing the movement of a nucleic acid comprising the polymorphic variant in polyacrylamide gels containing a gradient of denaturant, e.g., denaturing gradient gel electrophoresis (DGGE) (Myers et al (1985) Nature 313:495). When DGGE is used as the method of analysis, DNA will be modified to ensure that it does not completely denature, for example by adding a GC clamp of approximately 40 by of high-melting GC-rich DNA by PCR. In other embodiments, a temperature gradient may be used in place of a denaturing agent gradient to identify differences in the mobility of control and sample DNA (Rosenbaum and Reissner (1987) Biophys Chem 265:1275).

In another embodiment, identification of the polymorphic variant is carried out using an oligonucleotide ligation assay (OLA), as described, e.g., in U.S. Pat. No. 4,998,617 and in Landegren, U. et al., Science 241:1077-1080 (1988). The OLA protocol uses two oligonucleotides which are designed to be capable of hybridizing to abutting sequences of a single strand of a target. One of the oligonucleotides is linked to a separation marker, e.g, biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using a biotin ligand, such as avidin. Nickerson, D. A. et al. have described a nucleic acid detection assay that combines attributes of PCR and OLA (Nickerson, D. A. et al., Proc. Natl. Acad. Sci. (U.S.A.) 87:8923-8927 (1990). In this method, PCR is used to achieve the exponential amplification of target DNA which is then detected using OLA.

Several techniques based on this OLA method have been developed and can be used to detect specific polymorphic variants of a gene. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each OLA reaction can be detected using hapten specific antibodies that are differently labeled, for example, with enzyme reporters such as alkaline phosphatase or horseradish peroxidase. This system permits the detection of the two alleles using a high throughput format that leads to the production of two different colors.

Polymorphic variants may also be identified using methods for detecting single nucleotide polymorphisms. Because single nucleotide polymorphisms constitute sites of variation flanked by regions of invariant sequence, their analysis requires no more than the determination of the identity of the single nucleotide present at the site of variation and it is unnecessary to determine a complete gene sequence for each patient. Several methods have been developed to facilitate the analysis of such single nucleotide polymorphisms.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a subject. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data

In another embodiment, a solution-based method is used for determining the identity of a polymorphic variant. Cohen, D. et al. (French Patent 2,650,840; PCT Publication No. WO 91/02087). As in the Mundy method of U.S. Pat. No. 4,656,127, a primer is employed that is complementary to allelic sequences immediately 3′ to a polymorphic site. The method determines the identity of the nucleotide at that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet et al. (PCT Publication No. WO 92/15712). The method uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase.

Recently, several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990), Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)).

Since the T373N and L107X Sirt1 polymorphic variant are located in an exon, the identity of the polymorphic variant can be determined by analyzing the molecular structure of the mRNA, pre-mRNA, or cDNA. The molecular structure can be determined using any of the above described methods for determining the molecular structure of the genomic DNA, e.g., sequencing and SSCP. In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

Additional methods may be used for determining the identity of a polymorphic variant located in the coding region of a gene. For example, identification of a polymorphic variant which encodes a protein having a sequence variation can be performed using an antibody that specifically recognizes the protein variant, for example, using immunohistochemistry, immunoprecipitation or immunoblotting techniques. Antibodies to protein variants may be prepared according to methods known in the art and as described herein.

In certain embodiments, polymorphic variants may be detected by determining variations in sirtuin protein expression and/or activity. The expression level (i.e., abundance), expression pattern (e.g., temporal or spatial expression pattern, which includes subcellular localization, cell type specificity), size, sequence, association with other cellular constituents, etc., of Sirt1 in a sample obtained from a subject may be determined and compared with a control, e.g., the expression level or expression pattern that would be expected in a sample obtained from a normal subject.

In general, such detection and/or comparison may be performed using any of a number of suitable methods known in the art including, but not limited to, immunoblotting (Western blotting), immunohistochemistry, ELISA, radioimmunoassay, protein chips (e.g., comprising antibodies to the relevant proteins), mass spectrometry, etc. Historical data (e.g., the known expression level, activity, expression pattern, or size in the normal population) may be used for purposes of the comparison. Such methods may utilize Sirt1 antibodies that can distinguish between Sirt1 variants that differ at sites encoded by polymorphic variants. In certain embodiments, the Sirt1 antibody binds to a Sirt1 epitope that includes amino acid 107. In certain embodiments, the Sirt1 antibody distinguishes between Sirt1 having a leucine at position at 107 and a proline at position 107.

Generally applicable methods for producing antibodies are well known in the art and are described extensively in references cited above, e.g., Current Protocols in Immunology and Using Antibodies: A Laboratory Manual. It is noted that antibodies can be generated by immunizing animals (or humans) either with a full length polypeptide, a partial polypeptide, fusion protein, or peptide (which may be conjugated with another moiety to enhance immunogenicity). The specificity of the antibody will vary depending upon the particular preparation used to immunize the animal and on whether the antibody is polyclonal or monoclonal. For example, if a peptide is used the resulting antibody will bind only to the antigenic determinant represented by that peptide. It may be desirable to develop and/or select antibodies that specifically bind to particular regions of Sirt1, such as a region comprising amino acid 107. Such specificity may be achieved by immunizing the animal with peptides or polypeptide fragments that correspond to the desired region of Sirt1. For example, a peptide of about 15 to 50 consecutive amino acids of Sirt1, including amino acid 107, may be used as an immunogen. Alternately, a panel of monoclonal antibodies can be screened to identify those that specifically bind to the desired region of Sirt1. Antibodies that specifically bind to antigenic determinants that comprise a region encoded by a polymorphic site of Sirt1 are useful in accordance with the methods described herein. According to certain embodiments, such antibodies are able to distinguish between Sirt1 polypeptides that differ by a single amino acid. Any of the antibodies described herein may be labeled. The methods described herein may also utilize panels of antibodies able to specifically bind to a variety of polymorphic variants of Sirt1. In general, preferred antibodies will possess high affinity, e.g., a K_{d} of <200 nM, and preferably, of <100 nM for a specific polymorphic variant of Sirt1. Exemplary antibodies do not show significant reactivity (e.g., less than about 50%, 25%, 10%, 5%, 1%, or less, cross reactivity) with a different Sirt1 polymorphic variant or wild-type Sirt1.

In other embodiments, polymorphic variants may be determined by determining a change in level of activity of a Sirt1 protein. Such activity may be measured in a biological sample obtained from a subject. Methods for measuring Sirt1 activity, e.g., deacetylase activity, are known in the art and are further described in the Exemplification section herein.

### 3. Methods of Diagnosis and Prognosis

Provided herein are methods for diagnosis and prognosis of sirtuin mediated diseases and disorders, particularly Sirt1 mediated diseases and disorders, in patients carrying a T373N or L107X Sirt1 polymorphic variant. The methods disclosed herein may be used, for example, to identify a subject suffering from or susceptible to a sirtuin mediated disease or disorder, to identify a subject that would benefit from treatment with a sirtuin modulating compound, to predict the immediacy of onset and/or severity of a sirtuin mediated disease or disorder, to evaluate a subject's risk of developing a sirtuin mediated disease or disorder, to determine appropriate dosage and/or treatment regimens for subjects having one or more Sirt1 polymorphic variants, to determine the responsiveness of an individual with a sirtuin mediated disease or disorder to treatment with a sirtuin modulating compound, and/or to design individualized therapeutic treatments based on the presence or absence of one or more polymorphic variants in a subject.

A sirtuin protein refers to a member of the sirtuin deacetylase protein family, or preferably to the sir2 family, which include human Sirt1 (GenBank Accession No. NM_{—}012238 and NP_{—}036370 (or AF083106)), SIRT2 (GenBank Accession No. NM_{—}012237, NM_{—}030593, NP_{—}036369, NP_{—}085096, and AF083107), SIRT3, SIRT4, SIRT5, SIRT6 and SIRT7 (Brachmann et al. (1995) Genes Dev. 9:2888 and Frye et al. (1999) BBRC 260:273) proteins.

The distribution of one or more Sirt1 polymorphic variants in a large number of individuals exhibiting particular markers of disease status or drug response may be determined by any of the methods described above and compared with the distribution of polymorphic variants in patients that have been matched for age, ethnic origin, and/or any other statistically or medically relevant parameters, who exhibit quantitatively or qualitatively different status markers. Correlations are achieved using any method known in the art, including nominal logistic regression, chi square tests or standard least squares regression analysis. In this manner, it is possible to establish statistically significant correlations between particular polymorphic variants and particular disease statuses (given in p values). It is further possible to establish statistically significant correlations between particular polymorphic variants and changes in disease status or drug response such as would result, e.g., from particular treatment regimens. In this manner, it is possible to correlate polymorphic variants with responsivity to particular treatments.

In certain embodiments, a panel of polymorphic variants may be defined that predict the risk of a sirtuin mediated disease or disorder and/or predict drug response to a sirtuin modulating compound. This predictive panel is then used for genotyping of patients on a platform that can genotype multiple polymorphic variants, such as SNPs, at the same time (Multiplexing). Preferred platforms include, for example, gene chips (Affymetrix) or the Luminex LabMAP reader. The subsequent identification and evaluation of a patient's genotype and/or haplotype can then help to guide specific and individualized therapy.

For example, the methods disclosed herein permit the identification of patients exhibiting polymorphic variants that are associated with an increased risk for adverse drug reactions (ADR). In such cases, dose of a sirtuin modulating compound can be lowered to reduce or eliminate the risk for ADR. Also if a patient's response to drug administration is particularly high (e.g., the patient does not metabolize the drug well), the dose of the sirtuin modulating compound can be lowered to avoid the risk of ADR. In turn if the patient's response to drug administration is low (e.g., the patient is a particularly high metabolizer of the drug), and there is no evident risk of ADR, the dose of the sirtuin modulating compound can be raised to an efficacious level.

The ability to predict a patient's individual drug response to a sirtuin modulating compound permits formulation of sirtuin modulating compounds to be tailored in a way that suits the individual needs of the patient or class of patients (e.g., low/high responders, poor/good metabolizers, ADR prone patients, etc.). For example, formulations of sirtuin modulating compounds may be individualized to encompass different sirtuin modulating compounds, different doses of the drug, different modes of administration, different frequencies of administration, and different pharmaceutically acceptable carriers. The individualized sirtuin modulating formulation may also contain additional substances that facilitate the beneficial effects and/or diminish the risk for ADR (Folkers et al. 1991, U.S. Pat. No. 5,316,765).

The present invention also provides a method for determining whether a subject has a sirtuin mediated disease or disorder or a pre-disposition to a sirtuin mediated disease or disorder. Such methods may comprise, for example, obtaining information about the presence or absence of a T373N or L107X Sirt1 polymorphic variant. Other information such as phenotypic information about said subject may also be obtained. This information may then be analyzed to correlate the T373N or L107X Sirt1 polymorphic variant with a risk of developing a sirtuin mediated disease or disorder, severity of a sirtuin mediated disease or disorder, optimal therapeutic treatments, dosage schedules, etc. The method may further comprise the step of recommending a particular treatment for treating or preventing the sirtuin mediated disease or disorder.

In another embodiment, the invention provides a method for predicting the lifespan of an individual. The method comprises determining the presence or absence of a T373N or L107X Sirt1 polymorphic variant in a subject, and using the information to calculate a predicted lifespan for said individual. Additional information, such as, one or more additional lifespan factors including age, gender, weight, smoking, disease, etc. may be used in conjunction with the Sirt1 haplotype to calculate the predicted lifespan. Such information can be used, for example, in association with pricing and issuance of insurance policies such as life insurance policies.

In another embodiment, the invention provides a method for evaluating stem cells to be used in association with various cell therapies and methods of treatment using such stem cells. For example, stem cells having a more favorable Sirt1 haplotype may be selected over stem cells having a less favorable Sirt1 haplotype for cell therapy. Stem cells include any type of stem cells suitable for cell therapy including embryonic stem cells. Such stem cells may be used for treating a variety of diseases and disorders including, for example, Parkinson's disease, Huntington's disease and Alzheimer's disease. Exemplary methods may comprise, for example: identifying the presence or absence of one or more polymorphic variants in one or more stem cell samples, identifying a stem cell sample having a favorable Sirt1 haplotype, and using the identified population of stem cells in association with cell therapy for treatment of a disease or disorder that would benefit from the cell therapy.

### 4. Pharmacogenetic and Pharmacogenomic Uses

In various embodiments, knowledge of the status of the T373N or L107X Sirt1 polymorphic variant can be used to help identify patients most suited to therapy with particular pharmaceutical agents (this is often termed “pharmacogenetics”). Pharmacogenetics can also be used in pharmaceutical research to assist the drug selection process. Polymorphisms are used in mapping the human genome and to elucidate the genetic component of diseases. The following references give further background details on pharmacogenetics and other uses of polymorphism detection: Linder et al. (1997), Clinical Chemistry, 43, 254; Marshall (1997), Nature Biotechnology, 15, 1249; International Patent Application WO 97/40462, Spectra Biomedical; and Schafer et al. (1998), Nature Biotechnology, 16, 33.

Pharmacogenetics is generally regarded as the study of genetic variation that gives rise to differing response to drugs, while pharmacogenomics is the broader application of genomic technologies to new drug discovery and further characterization of older drugs. Pharmacogenetics considers one or at most a few genes of interest, while pharmacogenomics considers the entire genome. Much of current clinical interest is at the level of pharmacogenetics, involving variation in genes involved in drug metabolism with a particular emphasis on improving drug safety.

Pharmacogenomics is the science of utilizing human genetic variation to optimize patient treatment and drug design and discovery. An individual's genetic make up affects each stage of drug response: absorption, metabolism, transport to the target molecule, structure of the intended and/or unintended target molecules, degradation and excretion.

Pharmacogenomics provides the basis for a new generation of personalized pharmaceuticals, the targeting of drug therapies to genetic subpopulations. Currently drugs are developed to benefit the widest possible populations. However the variations in drug reactions attributed to genetic variation are increasingly being taken into account when developing new drugs. There are multiple benefits to such an approach to drug design. The development of genetic tests may reduce the need for the standard trial and error method of drug prescription. Targeted prescriptions would further reduce the incidence of adverse drug reactions, which are estimated to be the fifth ranking cause of death in the United States. Furthermore, dosage decisions can be made on a more informed basis than currently used parameters such as age, sex and weight. Drug discovery and approval processes will likely be speeded up by the specific genetic targeting of candidate drugs. Moreover, this may allow the revival of previously failed candidate drugs. Overall it is expected that the development of personalized pharmaceuticals will reduce the costs of healthcare.

The present disclosure provides methods for analyzing Sirt1 gene polymorphisms, particularly T373N or L107X Sirt1 polymorphic variants, of a subject in a variety of settings that may be before, during or after a medical event including, but not limited to, treatment with an approved drug, treatment with an experimental drug during a clinical trial, trauma, surgery, preventative therapy, vaccination, drug dosing determination, drug efficacy determination, progress or course of therapy with a drug, monitoring disease stage or status or progression, aging, drug addiction, weight loss or gain, cardiovascular or other cardiac-related events, reactions to treatment with a drug, exposure to radiation or other environmental events, exposure to weightlessness or other environmental conditions, exposure to chemical or biological agents (both natural and man-made), and/or diet (ingestion of foodstuffs). In addition, the present invention provides a database of Sirt1 gene polymorphism data for a subject or group of subjects obtained before, during or after a medical event. In one embodiment, the Sirt1 gene polymorphism data obtained according to the present invention is from a subject involved in a clinical trial. In another embodiment, the Sirt1 gene polymorphism data identifies any gene, or collection of genes, that undergoes a change in its level of expression without regard for the function of the encoded protein or association of the gene with any particular function, pathway, disease or other attribute other than its ability to be detected.

In another embodiment, other gene or genes of interest may be known to have an association with the gene expression profile of the subject or the medical event of interest. In one embodiment, for example, another gene known to predispose a subject to a particular disease when expressed, may be monitored before any symptoms are present in the subject to establish a baseline expression level in that subject. Monitoring the Sirt1 gene polymorphisms in the patient may be used to treat, suppress or prevent diseases or disorders related to aging or stress, diabetes, cancer, obesity, neurodegenerative diseases, diseases or disorders associated with mitochondrial dysfunction, chemotherapeutic induced neuropathy, neuropathy associated with an ischemic event, ocular diseases and/or disorders, cardiovascular disease, blood clotting disorders, inflammation, and/or flushing, etc. and other chronic and non-chronic diseases as detailed in The Merck Manual of Diagnosis and Therapy (Beers & Berkow, Eds.).

Adverse drug reactions are a principal cause of the low success rate of drug development programs (less than one in four compounds that enter human clinical testing is ultimately approved for use by the U.S. Food and Drug Administration (FDA)). Drug-induced disease or toxicity presents a unique series of challenges to drug developers, as these reactions are often not predictable from preclinical studies and may not be detected in early clinical trials involving small numbers of subjects. When such effects are detected in later stages of clinical development they often result in termination of a drug development program. When a drug is approved despite some toxicity, its clinical use is frequently severely constrained by the possible occurrence of adverse reactions in even a small group of patients. The likelihood of such a compound becoming a first line therapy is small (unless there are no competing products). Clinical trials that use this invention may allow for improved predictions of possible toxic reactions in studies involving a small number of subjects. The methods of this invention offer a quickly derived prediction of likely future toxic effects of an intervention.

Absorption is the first pharmacokinetic parameter to consider when determining variation in drug response. The actual effects of absorption on an individual or group of individuals may be quickly determined using this invention.

Once a drug or candidate therapeutic intervention is absorbed, injected or otherwise enters the bloodstream it is distributed to various biological compartments via the blood. The drug may exist free in the blood, or, more commonly, may be bound with varying degrees of affinity to plasma proteins. One classic source of variation in drug response is attributable to amino acid polymorphisms in serum albumin, which affect the binding affinity of drugs such as warfarin. Consequent variation in levels of free warfarin has a significant effect on the degree of anticoagulation. From the blood a compound diffuses into and is retained in interstitial and cellular fluids of different organs to different degrees. The invention allows for use of genetic haplotyping to be used instead of measurements of the proteins reducing the time and complexity of measurements.

Once absorbed by the gastrointestinal tract, compounds encounter detoxifying and metabolizing enzymes in the tissues of the gastrointestinal system. Many of these enzymes are known to be polymorphic in man and account for well studied variation in pharmacokinetic parameters of many drugs. Subsequently compounds enter the hepatic portal circulation in a process commonly known as first pass. The compounds then encounter a vast array of xenobiotic detoxifying mechanisms in the liver, including enzymes that are expressed solely or at high levels only in liver. These enzymes include the cytochrome P450s, glucuronlytransferases, sulfotransferases, acetyltransferases, methyltransferases, the glutathione conjugating system, flavine monooxygenases, and other enzymes known in the art.

Biotransformation reactions in the liver often have the effect of converting lipophilic compounds into hydrophilic molecules that are then more readily excreted. Variation in these conjugation reactions may affect half-life and other pharmacokinetic parameters. It is important to note that metabolic transformation of a compound not infrequently gives rise to a second or additional compounds that have biological activity greater than, less than, or different from that of the parent compound. Metabolic transformation may also be responsible for producing toxic metabolites.

Genomic expressions can be a precursor to medical events such as clinical responses. The methods of the present invention allow for a prediction of clinical responses on an individual or generally across a population due to an event or intervention. A “Medical Event” is any occurrence that may result in death, may be life-threatening, may require hospitalization, or prolongation of existing hospitalization, may result in persistent or significant disability/incapacity, may be a congenital anomaly/birth defect, may require surgical or non-surgical intervention to prevent one or more of the outcomes listed in this definition, may result in a change in clinical symptoms, or otherwise may result in change in the health of an individual or group of individuals whether naturally or as a result of human intervention.

Different events or interventions may present different responses in gene expression within a subject or between subjects. The invention allows the gene expression responses from differing interventions to be compared to help determine relative effectiveness and toxicity among different interventions and medical events and interventions, including those described in Behrman: Nelson Textbook of Pediatrics, Braunwald: Heart Disease: A Textbook of Cardiovascular Medicine, Brenner: Brenner & Rector's The Kidney, Canale: Campbell's Operative Orthopaedics, Cotran: Robbins Pathologic Basis of Disease, Cummings et al: Otolaryngology—Head and Neck Surgery, DeLee: DeLee and Drez's Orthopaedic Sports Medicine, Duthie: Practice of Geriatric, Feldman: Sleisenger & Fordtran's Gastrointestinal and Liver Disease, Ferri: Ferri's Clinical Advisor, Ferri: Practical Guide to the Care of the Medical Patient, Ford: Clinical Toxicology, Gabbe: Obstetrics: Normal and Problem Pregnancies, Goetz: Textbook of Clinical Neurology, Goldberger: Clinical Electrocardiography, Goldman: Cecil Textbook of Medicine, Grainger: Grainger & Allison's Diagnostic Radiology, Habif: Clinical Dermatology: Color Guide to Diagnosis and Therapy, Hoffman: Hematology: Basic Principles and Practice, Jacobson: Psychiatric Secrets, Johns Hopkins. The Harriet Lane Handbook, Larsen: Williams Textbook of Endocrinology, Long: Principles and Practices of Pediatric Infectious Disease, Mandell: Principles and Practice of Infectious Diseases, Marx: Rosen's Emergency Medicine Concepts and Clinical Practice, Middleton: Allergy: Principles and Practice, Miller: Anesthesia, Murray & Nadel: Textbook of Respiratory Medicine, Noble: Textbook of Primary Care Medicine, Park: Pediatric Cardiology for Practitioners, Pizzorno: Textbook of Natural Medicine, Rakel: Conn's Current Therapy, Rakel: Textbook of Family Medicine, Ravel: Clinical Laboratory Medicine, Roberts: Clinical Procedures in Emergency Medicine, Ruddy: Kelley's Textbook of Rheumatology, Ryan: Kistner's Gynecology and Women's Health Townsend: Sabiston Textbook of Surgery, Yanoff: Ophthalmology, and Walsh: Campbell's Urology.

The terms “disease” or “condition” are commonly recognized in the art and designate the presence of signs and/or symptoms in an individual or patient that are generally recognized as abnormal. Diseases or conditions may be diagnosed and categorized based on pathological changes. Signs may include any objective evidence of a disease such as changes that are evident by physical examination of a patient or the results of diagnostic tests. Symptoms are subjective evidence of disease or a patient's condition, i.e. the patient's perception of an abnormal condition that differs from normal function, sensation, or appearance, which may include, without limitations, physical disabilities, morbidity, pain, and other changes from the normal condition experienced by an individual. Various diseases or conditions include, but are not limited to; those categorized in standard textbooks of medicine including, without limitation, textbooks of nutrition, allopathic, homeopathic, and osteopathic medicine. In certain aspects of this invention, the disease or condition is selected from the group consisting of the types of diseases listed in standard texts such as Harrison's Principles of Internal Medicine, 14.sup.th Edition (Fauci et al, Eds., McGraw Hill, 1997), or Robbins Pathologic Basis of Disease, 6.sup.th Edition (Cotran et al, Ed. W B Saunders Co., 1998), or the Diagnostic and Statistical Manual of Mental Disorders: DSM-IV, 4.sup.th Edition, (American Psychiatric Press, 1994), or other texts described below.

The term “suffering from a disease or condition” means that a person is either presently subject to the signs and symptoms, or is more likely to develop such signs and symptoms than a normal person in the population. Thus, for example, a person suffering from a condition can include a developing fetus, a person subject to a treatment or environmental condition which enhances the likelihood of developing the signs or symptoms of a condition, or a person who is being given or will be given a treatment which increase the likelihood of the person developing a particular condition. For example, tardive dyskinesia is associated with long-term use of anti-psychotics; dyskinesias, paranoid ideation, psychotic episodes and depression have been associated with use of L-dopa in Parkinson's disease; and dizziness, diplopia, ataxia, sedation, impaired mentation, weight gain, and other undesired effects have been described for various anticonvulsant therapies, alopecia and bone marrow suppression are associated with cancer chemotherapeutic regimens, and immunosuppression is associated with agents to limit graft rejection following transplantation. Thus, methods of the present invention which relate to treatments of patients (e.g., methods for selecting a treatment, selecting a patient for a treatment, and methods of treating a disease or condition in a patient) can include primary treatments directed to a presently active disease or condition, secondary treatments which are intended to cause a biological effect relevant to a primary treatment, and prophylactic treatments intended to delay, reduce, or prevent the development of a disease or condition, as well as treatments intended to cause the development of a condition different from that which would have been likely to develop in the absence of the treatment.

The term “intervention” refers to a process that is intended to produce a beneficial change in the condition of a mammal, e.g., a human, often referred to as a patient. A beneficial change can, for example, include one or more of: restoration of function, reduction of symptoms, limitation or retardation of progression of a disease, disorder, or condition or prevention, limitation or retardation of deterioration of a patient's condition, disease or disorder. Such intervention can involve, for example, nutritional modifications, administration of radiation, administration of a drug, surgery, behavioral modifications, and combinations of these, among others.

The term “intervention” includes administration of “drugs” and “candidate therapeutic agents”. A drug is a chemical entity or biological product, or combination of chemical entities or biological products, administered to a person to treat or prevent or control a disease or condition. The chemical entity or biological product is preferably, but not necessarily a low molecular weight compound, but may also be a larger compound, for example, an oligomer of nucleic acids, amino acids, or carbohydrates including without limitation proteins, oligonucleotides, ribozymes, DNAzymes, glycoproteins, lipoproteins, and modifications and combinations thereof. A biological product is preferably a monoclonal or polyclonal antibody or fragment thereof such as a variable chain fragment; cells; or an agent or product arising from recombinant technology, such as, without limitation, a recombinant protein, recombinant vaccine, or DNA construct developed for therapeutic, e.g., human therapeutic, use. The term may include, without limitation, compounds that are approved for sale as pharmaceutical products by government regulatory agencies (e.g., U.S. Food and Drug Administration (USFDA or FDA), European Medicines Evaluation Agency (EMEA), and a world regulatory body governing the International Conference of Harmonization (ICH) rules and guidelines), compounds that do not require approval by government regulatory agencies, food additives or supplements including compounds commonly characterized as vitamins, natural products, and completely or incompletely characterized mixtures of chemical entities including natural compounds or purified or partially purified natural products. The term “drug” as used herein is synonymous with the terms “medicine”, “pharmaceutical product”, or “product”. Most preferably the drug is approved by a government agency for treatment of a specific disease or condition. The term “candidate therapeutic agent” refers to a drug or compound that is under investigation, either in laboratory or human clinical testing for a specific disease, disorder, or condition.

The intervention may involve either positive selection or negative selection or both, meaning that the selection can involve a choice that a particular intervention would be an appropriate method to use and/or a choice that a particular intervention would be an inappropriate method to use. Thus, in certain embodiments, the presence of the at least one Sirt1 haplotype may be indicative that the treatment will be effective or otherwise beneficial (or more likely to be beneficial) in the patient. Stating that the treatment will be effective means that the probability of beneficial therapeutic effect is greater than in a person not having the appropriate presence or absence of a particular Sirt1 haplotype. In other embodiments, the presence of the at least one Sirt1 haplotype is indicative that the treatment will be ineffective or contra-indicated for the patient. For example, a treatment may be contra-indicated if the treatment results, or is more likely to result, in undesirable side effects, or an excessive level of undesirable side effects. A determination of what constitutes excessive side-effects will vary, for example, depending on the disease or condition being treated, the availability of alternatives, the expected or experienced efficacy of the treatment, and the tolerance of the patient. As for an effective treatment, this means that it is more likely that desired effect will result from the treatment administration in a patient showing a Sirt1 haplotype consistent with the desired clinical outcome. Also in preferred embodiments, the presence of the at least Sirt1 haplotype is indicative that the treatment is both effective and unlikely to result in undesirable effects or outcomes, or vice versa (is likely to have undesirable side effects but unlikely to produce desired therapeutic effects).

The invention may be useful in predicting a patient's tolerance to an intervention. In reference to response to a treatment, the term “tolerance” refers to the ability of a patient to accept a treatment, based, e.g., on deleterious effects and/or effects on lifestyle. Frequently, the term principally concerns the patients' perceived magnitude of deleterious effects such as nausea, weakness, dizziness, and diarrhea, among others. Such experienced effects can, for example, be due to general or cell-specific toxicity, activity on non-target cells, cross-reactivity on non-target cellular constituents (non-mechanism based), and/or side effects of activity on the target cellular substituents (mechanism based), or the cause of toxicity may not be understood. In any of these circumstances one may identify an association between the undesirable effects and Sirt1 haplotype.

Adverse responses to drugs constitute a major medical problem, as shown in two recent meta-analyses (Lazarou et al, “Incidence of Adverse Drug Reactions in Hospitalized Patients: A Meta-Analysis of Prospective Studies”, 279 JAMA 1200-1205 (1998); and Bonn, “Adverse Drug Reactions Remain a Major Cause of Death”, 351 LANCET 1183 (1998). An estimated 2.2 million hospitalized patients in the United Stated had serious adverse drug reactions in 1994, with an estimated 106,000 deaths (Lazarou et al.). To the extent that some of these adverse events are predictable based on changes in RNA expression, the identification of changes that are predictive of such effects will allow for more effective and safer drug use.

The disclosed methods also have uses in the area of eliminating treatments. The phrase “eliminating a treatment” refers to removing a possible treatment from consideration, e.g., for use with a particular patient based on one or more changes in Sirt1 haplotype, or to stopping the administration of a treatment which was in the course of administration.

Also in preferred embodiments, the method of selecting a treatment involves selecting a method of administration of a compound, combination of compounds, or pharmaceutical composition, for example, selecting a suitable dosage level and/or frequency of administration, and/or mode of administration of a compound. The method of administration can be selected to provide better, preferably maximum therapeutic benefit. In this context, “maximum” refers to an approximate local maximum based on the parameters being considered, not an absolute maximum. The term “suitable dosage level” refers to a dosage level which provides a therapeutically reasonable balance between pharmacological effectiveness and deleterious effects. Often this dosage level is related to the peak or average serum levels resulting from administration of a drug at the particular dosage level.

In certain specific embodiments, if a patient has a L107X Sirt1 polymorphic variant, that patient should receive a higher dose of a sirtuin-activating compound than a patient that has leucine at amino acid 107 of Sirt1. In certain specific embodiments, if a patient has a T373N Sirt1 polymorphic variant, that patient should receive a higher dose of a sirtuin-activating compound than a patient that has thymine at nucleotide 373 of Sirt1.

Similarly, a “frequency of administration” refers to how often in a specified time period a treatment is administered, e.g., once, twice, or three times per day, every other day, once per week, etc. For a drug or drugs, the frequency of administration is generally selected to achieve a pharmacologically effective average or peak serum level without excessive deleterious effects (and preferably while still being able to have reasonable patient compliance for self-administered drugs). Thus, it is desirable to maintain the serum level of the drug within a therapeutic window of concentrations for the greatest percentage of time possible without such deleterious effects as would cause a prudent physician to reduce the frequency of administration for a particular dosage level.

In certain specific embodiments, if a patient has a L107X Sirt1 polymorphic variant, that patient should receive a more frequent administration of a sirtuin-activating compound than a patient that has leucine at amino acid 107 of Sirt1. In certain specific embodiments, if a patient has a T373N Sirt1 polymorphic variant, that patient should receive more frequent administration of a sirtuin-activating compound than a patient that has thymine at nucleotide 373 of Sirt1.

Thus, in connection with the administration of a drug, a drug which is “effective against” a disease or condition indicates that administration in a clinically appropriate manner results in a beneficial effect for at least a statistically significant fraction of patients, such as a improvement of symptoms, a cure, a reduction in disease load, reduction in tumor mass or cell numbers, extension of life, improvement in quality of life, or other effect generally recognized as positive by medical doctors familiar with treating the particular type of disease or condition.

Effectiveness is measured in a particular population. In conventional drug development the population is generally every subject who meets the enrollment criteria (i.e. has the particular form of the disease or condition being treated). It is an aspect of the present invention that segmentation of a study population by genetic criteria can provide the basis for identifying a subpopulation in which a drug is effective against the disease or condition being treated.

The term “deleterious effects” refers to physical effects in a patient caused by administration of a treatment which are regarded as medically undesirable. Thus, for example, deleterious effects can include a wide spectrum of toxic effects injurious to health such as death of normally functioning cells when only death of diseased cells is desired, nausea, fever, inability to retain food, dehydration, damage to critical organs such as arrhythmias, renal tubular necrosis, fatty liver, or pulmonary fibrosis leading to coronary, renal, hepatic, or pulmonary insufficiency among many others. In this regard, the term “adverse reactions” refers to those manifestations of clinical symptomology of pathological disorder or dysfunction induced by administration of a drug, agent, or candidate therapeutic intervention. In this regard, the term “contraindicated” means that a treatment results in deleterious effects such that a prudent medical doctor treating such a patient would regard the treatment as unsuitable for administration. Major factors in such a determination can include, for example, availability and relative advantages of alternative treatments, consequences of non-treatment, and permanency of deleterious effects of the treatment.

It is recognized that many treatment methods, e.g., administration of certain compounds or combinations of compounds, may produce side-effects or other deleterious effects in patients. Such effects can limit or even preclude use of the treatment method in particular patients, or may even result in irreversible injury, disorder, dysfunction, or death of the patient. Thus, in certain embodiments, the variance information is used to select both a first method of treatment and a second method of treatment. Usually the first treatment is a primary treatment which provides a physiological effect directed against the disease or condition or its symptoms. The second method is directed to reducing or eliminating one or more deleterious effects of the first treatment, e.g., to reduce a general toxicity or to reduce a side effect of the primary treatment. Thus, for example, the second method can be used to allow use of a greater dose or duration of the first treatment, or to allow use of the first treatment in patients for whom the first treatment would not be tolerated or would be contra-indicated in the absence of a second method to reduce deleterious effects or to potentiate the effectiveness of the first treatment.

In a related aspect, the instant disclosure provides a method for selecting a method of treatment for a patient suffering from a disease or condition by comparing changes in gene expression to pharmacokinetic parameters, or organ and tissue damage, or inordinate immune response, which are indicative of the effectiveness or safety of at least one method of treatment.

Similar to the above aspect, in preferred embodiments, at least one method of treatment involves the administration of a compound effective in at least some patients with a disease or condition; the presence or absence of the at least one change in gene expression is indicative that the treatment will be effective in the patient; and/or the presence or absence of the at least one change in gene expression is indicative that the treatment will be ineffective or contra-indicated in the patient; and/or the treatment is a first treatment and the presence or absence of the at least one change in gene expression is indicative that a second treatment will be beneficial to reduce a deleterious effect or potentiate the effectiveness of the first treatment; and/or the at least one treatment is a plurality of methods of treatment. For a plurality of treatments, preferably the selecting involves determining whether any of the methods of treatment will be more effective than at least one other of the plurality of methods of treatment. Yet other embodiments are provided as described for the preceding aspect in connection with methods of treatment using administration of a compound; treatment of various diseases, and variances in genetic expressions.

In addition to the basic method of treatment, often the mode of administration of a given compound as a treatment for a disease or condition in a patient is significant in determining the course and/or outcome of the treatment for the patient. Thus, the invention also provides a method for selecting a method of administration of a compound to a patient suffering from a disease or condition, by determining changes in gene expression where such presence or absence is indicative of an appropriate method of administration of the compound. Preferably, the selection of a method of treatment (a treatment regimen) involves selecting a dosage level or frequency of administration or route of administration of the compound or combinations of those parameters. In preferred embodiments, two or more compounds are to be administered, and the selecting involves selecting a method of administration for one, two, or more than two of the compounds, jointly, concurrently, or separately. As understood by those skilled in the art, such plurality of compounds may be used in combination therapy, and thus may be formulated in a single drug, or may be separate drugs administered concurrently, serially, or separately. Other embodiments are as indicated above for selection of second treatment methods, methods of identifying Sirt1 haplotypes, and methods of treatment as described for aspects above.

In another aspect, the invention provides a method for selecting a patient for administration of a method of treatment for a disease or condition, or of selecting a patient for a method of administration of a treatment, by analyzing Sirt1 haplotype as identified above in peripheral blood of a patient, where the Sirt1 haplotype is indicative that the treatment or method of administration that will be effective in the patient.

In one embodiment, the disease or the method of treatment is as described in aspects above, specifically including, for example, those described for selecting a method of treatment.

In another aspect, the invention provides a method for identifying patients with enhanced or diminished response or tolerance to a treatment method or a method of administration of a treatment where the treatment is for a disease or condition in the patient. The method involves correlating one or more Sirt1 haplotypes as identified in aspects above in a plurality of patients with response to a treatment or a method of administration of a treatment. The correlation may be performed by determining the one or more Sirt1 haplotypes in the plurality of patients and correlating the presence or absence of each of the changes (alone or in various combinations) with the patient's response to treatment. The Sirt1 haplotype(s) may be previously known to exist or may also be determined in the present method or combinations of prior information and newly determined information may be used. The enhanced or diminished response should be statistically significant, preferably such that p=0.10 or less, more preferably 0.05 or less, and most preferably 0.02 or less. A positive correlation between the presence of one or more Sirt1 haplotypes and an enhanced response to treatment is indicative that the treatment is particularly effective in the group of patients showing certain patterns of Sirt1 haplotypes. A positive correlation of the presence of the one or more expression changes with a diminished response to the treatment is indicative that the treatment will be less effective in the group of patients having those variances. Such information is useful, for example, for selecting or de-selecting patients for a particular treatment or method of administration of a treatment, or for demonstrating that a group of patients exists for which the treatment or method of treatment would be particularly beneficial or contra-indicated. Such demonstration can be beneficial, for example, for obtaining government regulatory approval for a new drug or a new use of a drug.

Preferred embodiments include drugs, treatments, variance identification or determination, determination of effectiveness, and/or diseases as described for aspects above or otherwise described herein.

In other embodiments, the correlation of patient responses to therapy according to Sirt1 haplotype is carried out in a clinical trial, e.g., as described herein according to any of the variations described. Detailed description of methods for associating variances with clinical outcomes using clinical trials is provided below. Further, in preferred embodiments the correlation of pharmacological effect (positive or negative) to Sirt1 haplotype in such a clinical trial is part of a regulatory submission to a government agency leading to approval of the drug. Most preferably the compound or compounds would not be approvable in the absence of this data.

As indicated above, in aspects of this invention involving selection of a patient for a treatment, selection of a method or mode of administration of a treatment, and selection of a patient for a treatment or a method of treatment, the selection may be positive selection or negative selection. Thus, the methods can include eliminating a treatment for a patient, eliminating a method or mode of administration of a treatment to a patient, or elimination of a patient for a treatment or method of treatment.

The present invention provides a method for treating a patient at risk for drug responsiveness, i.e., efficacy differences associated with pharmacokinetic parameters, and safety concerns, i.e. drug-induced disease, disorder, or dysfunction or diagnosed with organ failure or a disease associated with drug-induced organ failure. The methods include identifying such a patient and determining the patient's changes in genetic expressions. The patient identification can, for example, be based on clinical evaluation using conventional clinical metrics.

In a related aspect, the invention provides a method for identifying a patient for participation in a clinical trial of a therapy for the treatment of a disease, disorder, or dysfunction, or an associated drug-induced toxicity. The method involves determining the T373N or L107X Sirt1 polymorphic variant of a patient with (or at risk for) a disease, disorder, or dysfunction. The trial would then test the hypothesis that a statistically significant difference in response to a treatment can be demonstrated between two groups of patients based on Sirt1 polymorphic variant(s). Said response may be a desired or an undesired response. In a preferred embodiment, the treatment protocol involves a comparison of placebo vs. treatment response rates in two or more groups. For example a group with Sirt1 having leucine at amino acid 107 or thymine at nucleotide 373 may be compared to a group with Sirt1 having an amino acid other than leucine at position 107 or a nucleotide other than thymine at position 373.

In another preferred embodiment, patients in a clinical trial can be grouped (at the end of the trial) according to treatment response, and statistical methods can be used to compare Sirt1 polymorphic variants of the patients. For example responders can be compared to nonresponders, or patients suffering adverse events can be compared to those not experiencing such effects. Alternatively response data can be treated as a continuous variable and the ability of Sirt1 polymorphic variant status to predict response can be measured. In a preferred embodiment, patients who exhibit extreme responses are compared with all other patients or with a group of patients who exhibit a divergent extreme response. For example if there is a continuous or semi-continuous measure of treatment response (for example the Alzheimer's Disease Assessment Scale, the Mini-Mental State Examination or the Hamilton Depression Rating Scale) then the 10% of patients with the most favorable responses could be compared to the 10% with the least favorable, or the patients one standard deviation above the mean score could be compared to the remainder, or to those one standard deviation below the mean score. One useful way to select the threshold for defining a response is to examine the distribution of responses in a placebo group. If the upper end of the range of placebo responses is used as a lower threshold for an ‘outlier response’ then the outlier response group should be almost free of placebo responders. This is a useful threshold because the inclusion of placebo responders in a ‘true’ response group decreases the ability of statistical methods to detect changes in gene expression between responders and nonresponders.

In a related aspect, the invention provides a method for developing a disease management protocol that entails diagnosing a patient with a disease or a disease susceptibility, determining the Sirt1 polymorphic variant(s) of the patient and then selecting an optimal treatment based on the disease and the Sirt1 polymorphic variant(s). The disease management protocol may be useful in an education program for physicians, other caregivers or pharmacists; may constitute part of a drug label; or may be useful in a marketing campaign.

“Disease management protocol” or “treatment protocol” is a means for devising a therapeutic plan for a patient using laboratory, clinical and genetic data, including the patient's diagnosis and genotype. The protocol clarifies therapeutic options and provides information about probable prognoses with different treatments. The treatment protocol may provide an estimate of the likelihood that a patient will respond positively or negatively to a therapeutic intervention. The treatment protocol may also provide guidance regarding optimal drug dose and administration and likely timing of recovery or rehabilitation. A “disease management protocol” or “treatment protocol” may also be formulated for asymptomatic and healthy subjects in order to forecast future disease risks based on laboratory, clinical and gene expression variables. In this setting the protocol specifies optimal preventive or prophylactic interventions, including use of compounds, changes in diet or behavior, or other measures. The treatment protocol may include the use of a computer program.

In other embodiments of above aspects involving prediction of drug efficacy, the prediction of drug efficacy involves candidate therapeutic interventions that are known or have been identified to be affected by pharmacokinetic parameters, i.e. absorption, distribution, metabolism, or excretion. These parameters may be associated with hepatic or extra-hepatic biological mechanisms. Preferably the candidate therapeutic intervention will be effective in patients with the known changes in genetic expression but have a risk of drug ineffectiveness, i.e. nonresponsive to a drug or candidate therapeutic intervention.

In other embodiments, the above methods are used for or include identification of a safety or toxicity concern involving a drug-induced disease, disorder, or dysfunction and/or the likelihood of occurrence and/or severity of said disease, disorder, or dysfunction.

In other embodiments, the disclosed methods are suitable for identifying a patient with non-drug-induced disease, disorder, or dysfunction but with dysfunction related to aberrant enzymatic metabolism or excretion of endogenous biologically relevant molecules or compounds.

### 5. Methods of Treatment

Provided herein are methods of treating a Sirt1 mediated disease or disorder. A Sirt1 mediated disease or disorder refer to a disease, disorder or condition that is associated with a decrease in the level and/or activity of a Sirt1 protein. Examples of Sirt1 mediated diseases or disorders include, for example, aging, stress, diabetes, obesity, neurodegenerative diseases, cancer, chemotherapeutic induced neuropathy, neuropathy associated with an ischemic event, ocular diseases and/or disorders, cardiovascular disease, blood clotting disorders, inflammation, flushing, disease associated with abnormal mitochondrial activity, decreased muscle performance, decreased muscle ATP levels, or muscle tissue damage associated with hypoxia or ischemia.

Methods of treatment involve administering a pharmaceutically effective amount of a sirtuin activating compound to a subject having a T373N or L107X Sirt1 polymorphic variant. In exemplary embodiments, the subject has a T373C Sirt1 nucleic acid mutation or a L107P Sirt1 amino acid mutation. In exemplary embodiments the subject being treated is heterozygous for the T373N Sirt1 nucleic acid mutation or the L107P Sirt1 amino acid mutation. The sirtuin activating compound may activate the wild-type Sirt1 of a heterozygous patient, the Sirt1 comprising the T373N or L107X variant, or both. In certain embodiments, administration of a sirtuin activator activates a sirtuin other than Sirt1.

In certain embodiments, the methods of treatment involve first testing the subject to determine the identity of the nucleic acid residue at position 373 of the Sirt1 nucleic acid sequence or position 107 of the Sirt1 amino acid sequence. For example, using the methods described herein, it may be determined if a subject carries a T373N mutation in the Sirt1 nucleic acid sequence or a L107X mutation in the Sirt1 protein sequence. In an exemplary embodiment, the presence of a T373C mutation in the Sirt1 nucleic acid sequence or a L107P mutation in the Sirt1 protein sequence is identified.

In certain embodiments, the methods of treatment provided herein involve administering a sirtuin activating compound alone or in combination with other compounds. In one embodiment, a mixture of two or more sirtuin-modulating compounds may be administered to a subject in need thereof. In yet another embodiment, one or more sirtuin-modulating compounds may be administered with one or more therapeutic agents for the treatment or prevention of various diseases, including, for example, cancer, diabetes, neurodegenerative diseases, cardiovascular disease, blood clotting, inflammation, flushing, obesity, ageing, stress, etc. In various embodiments, combination therapies comprising a sirtuin-modulating compound may refer to (1) pharmaceutical compositions that comprise one or more sirtuin-modulating compounds in combination with one or more therapeutic agents (e.g., one or more therapeutic agents described herein); and (2) co-administration of one or more sirtuin-modulating compounds with one or more therapeutic agents wherein the sirtuin-modulating compound and therapeutic agent have not been formulated in the same compositions (but may be present within the same kit or package, such as a blister pack or other multi-chamber package; connected, separately sealed containers (e.g., foil pouches) that can be separated by the user; or a kit where the sirtuin modulating compound(s) and other therapeutic agent(s) are in separate vessels). When using separate formulations, the sirtuin-modulating compound may be administered at the same, intermittent, staggered, prior to, subsequent to, or combinations thereof, with the administration of another therapeutic agent.

### Aging/Stress

In one embodiment, the invention provides a method extending the lifespan of a cell, extending the proliferative capacity of a cell, slowing aging of a cell, promoting the survival of a cell, delaying cellular senescence in a cell, mimicking the effects of calorie restriction, increasing the resistance of a cell to stress, or preventing apoptosis of a cell, wherein the cell comprises a T373N or L107X Sirt1 polymorphic variant. The methods involve contacting the cell with a sirtuin-activating compound.

In one embodiment, cells that are intended to be preserved for long periods of time may be treated with a sirtuin-modulating compound that increases the level and/or activity of a sirtuin protein. The cells may be in suspension (e.g., blood cells, serum, biological growth media, etc.) or in tissues or organs. For example, blood collected from an individual for purposes of transfusion may be treated with a sirtuin-modulating compound that increases the level and/or activity of a sirtuin protein to preserve the blood cells for longer periods of time. Additionally, blood to be used for forensic purposes may also be preserved using a sirtuin-modulating compound that increases the level and/or activity of a sirtuin protein. Other cells that may be treated to extend their lifespan or protect against apoptosis include cells for consumption, e.g., cells from non-human mammals (such as meat) or plant cells (such as vegetables).

Sirtuin-modulating compounds that increase the level and/or activity of a sirtuin protein may also be applied during developmental and growth phases in mammals, plants, insects or microorganisms, in order to, e.g., alter, retard or accelerate the developmental and/or growth process.

In another embodiment, sirtuin-modulating compounds that increase the level and/or activity of a sirtuin protein may be used to treat cells useful for transplantation or cell therapy, including, for example, solid tissue grafts, organ transplants, cell suspensions, stem cells, bone marrow cells, etc. The cells or tissue may be an autograft, an allograft, a syngraft or a xenograft. The cells or tissue may be treated with the sirtuin-modulating compound prior to administration/implantation, concurrently with administration/implantation, and/or post administration/implantation into a subject. The cells or tissue may be treated prior to removal of the cells from the donor individual, ex vivo after removal of the cells or tissue from the donor individual, or post implantation into the recipient. For example, the donor or recipient individual may be treated systemically with a sirtuin-modulating compound or may have a subset of cells/tissue treated locally with a sirtuin-modulating compound that increases the level and/or activity of a sirtuin protein. In certain embodiments, the cells or tissue (or donor/recipient individuals) may additionally be treated with another therapeutic agent useful for prolonging graft survival, such as, for example, an immunosuppressive agent, a cytokine, an angiogenic factor, etc.

In yet other embodiments, cells may be treated with a sirtuin-modulating compound that increases the level and/or activity of a sirtuin protein in vivo, e.g., to increase their lifespan or prevent apoptosis. For example, skin can be protected from aging (e.g., developing wrinkles, loss of elasticity, etc.) by treating skin or epithelial cells with a sirtuin-modulating compound that increases the level and/or activity of a sirtuin protein. In an exemplary embodiment, skin is contacted with a pharmaceutical or cosmetic composition comprising a sirtuin-modulating compound that increases the level and/or activity of a sirtuin protein. Exemplary skin afflictions or skin conditions that may be treated in accordance with the methods described herein include disorders or diseases associated with or caused by inflammation, sun damage or natural aging. For example, the compositions find utility in the prevention or treatment of contact dermatitis (including irritant contact dermatitis and allergic contact dermatitis), atopic dermatitis (also known as allergic eczema), actinic keratosis, keratinization disorders (including eczema), epidermolysis bullosa diseases (including penfigus), exfoliative dermatitis, seborrheic dermatitis, erythemas (including erythema multiforme and erythema nodosum), damage caused by the sun or other light sources, discoid lupus erythematosus, dermatomyositis, psoriasis, skin cancer and the effects of natural aging. In another embodiment, sirtuin-modulating compounds that increase the level and/or activity of a sirtuin protein may be used for the treatment of wounds and/or burns to promote healing, including, for example, first-, second- or third-degree burns and/or thermal, chemical or electrical burns. The formulations may be administered topically, to the skin or mucosal tissue.

Topical formulations comprising one or more sirtuin-modulating compounds that increase the level and/or activity of a sirtuin protein may also be used as preventive, e.g., chemopreventive, compositions. When used in a chemopreventive method, susceptible skin is treated prior to any visible condition in a particular individual.

Sirtuin-modulating compounds may be delivered locally or systemically to a subject. In one embodiment, a sirtuin-modulating compound is delivered locally to a tissue or organ of a subject by injection, topical formulation, etc.

In another embodiment, a sirtuin-modulating compound that increases the level and/or activity of a sirtuin protein may be used for treating or preventing a disease or condition induced or exacerbated by cellular senescence in a subject; methods for decreasing the rate of senescence of a subject, e.g., after onset of senescence; methods for extending the lifespan of a subject; methods for treating or preventing a disease or condition relating to lifespan; methods for treating or preventing a disease or condition relating to the proliferative capacity of cells; and methods for treating or preventing a disease or condition resulting from cell damage or death. In certain embodiments, the method does not act by decreasing the rate of occurrence of diseases that shorten the lifespan of a subject. In certain embodiments, a method does not act by reducing the lethality caused by a disease, such as cancer.

In yet another embodiment, a sirtuin-modulating compound that increases the level and/or activity of a sirtuin protein may be administered to a subject in order to generally increase the lifespan of its cells and to protect its cells against stress and/or against apoptosis. It is believed that treating a subject with a compound described herein is similar to subjecting the subject to hormesis, i.e., mild stress that is beneficial to organisms and may extend their lifespan.

Sirtuin-modulating compounds that increase the level and/or activity of a sirtuin protein may be administered to a subject to prevent aging and aging-related consequences or diseases, such as stroke, heart disease, heart failure, arthritis, high blood pressure, and Alzheimer's disease. Other conditions that can be treated include ocular disorders, e.g., associated with the aging of the eye, such as cataracts, glaucoma, and macular degeneration. Sirtuin-modulating compounds that increase the level and/or activity of a sirtuin protein can also be administered to subjects for treatment of diseases, e.g., chronic diseases, associated with cell death, in order to protect the cells from cell death. Exemplary diseases include those associated with neural cell death, neuronal dysfunction, or muscular cell death or dysfunction, such as Parkinson's disease, Alzheimer's disease, multiple sclerosis, amniotropic lateral sclerosis, and muscular dystrophy; AIDS; fulminant hepatitis; diseases linked to degeneration of the brain, such as Creutzfeld-Jakob disease, retinitis pigmentosa and cerebellar degeneration; myelodysplasis such as aplastic anemia; ischemic diseases such as myocardial infarction and stroke; hepatic diseases such as alcoholic hepatitis, hepatitis B and hepatitis C; joint-diseases such as osteoarthritis; atherosclerosis; alopecia; damage to the skin due to UV light; lichen planus; atrophy of the skin; cataract; and graft rejections. Cell death can also be caused by surgery, drug therapy, chemical exposure or radiation exposure.

Sirtuin-modulating compounds that increase the level and/or activity of a sirtuin protein can also be administered to a subject suffering from an acute disease, e.g., damage to an organ or tissue, e.g., a subject suffering from stroke or myocardial infarction or a subject suffering from a spinal cord injury. Sirtuin-modulating compounds that increase the level and/or activity of a sirtuin protein may also be used to repair an alcoholic's liver.

### Cardiovascular Disease

In another embodiment, the invention provides a method for treating and/or preventing a cardiovascular disease by administering a sirtuin-modulating compound that increases the level and/or activity of a sirtuin protein to a subject having a T373N or L107X Sirt1 polymorphic variant.

Cardiovascular diseases that can be treated or prevented using the sirtuin-modulating compounds that increase the level and/or activity of a sirtuin protein include cardiomyopathy or myocarditis; such as idiopathic cardiomyopathy, metabolic cardiomyopathy, alcoholic cardiomyopathy, drug-induced cardiomyopathy, ischemic cardiomyopathy, and hypertensive cardiomyopathy. Also treatable or preventable using compounds and methods described herein are atheromatous disorders of the major blood vessels (macrovascular disease) such as the aorta, the coronary arteries, the carotid arteries, the cerebrovascular arteries, the renal arteries, the iliac arteries, the femoral arteries, and the popliteal arteries. Other vascular diseases that can be treated or prevented include those related to platelet aggregation, the retinal arterioles, the glomerular arterioles, the vasa nervorum, cardiac arterioles, and associated capillary beds of the eye, the kidney, the heart, and the central and peripheral nervous systems. The sirtuin-modulating compounds that increase the level and/or activity of a sirtuin protein may also be used for increasing HDL levels in plasma of an individual.

Yet other disorders that may be treated with sirtuin-modulating compounds that increase the level and/or activity of a sirtuin protein include restenosis, e.g., following coronary intervention, and disorders relating to an abnormal level of high density and low density cholesterol.

In one embodiment, a sirtuin-modulating compound that increases the level and/or activity of a sirtuin protein may be administered as part of a combination therapeutic with another cardiovascular agent. In one embodiment, a sirtuin-modulating compound that increases the level and/or activity of a sirtuin protein may be administered as part of a combination therapeutic with an anti-arrhythmia agent. In another embodiment, a sirtuin-modulating compound that increases the level and/or activity of a sirtuin protein may be administered as part of a combination therapeutic with another cardiovascular agent.

### Cell Death/Cancer

Sirtuin-modulating compounds that increase the level and/or activity of a sirtuin protein may be administered to subjects who have recently received or are likely to receive a dose of radiation or toxin, wherein the subject has a T373N or L107X Sirt1 polymorphic variant. In one embodiment, the dose of radiation or toxin is received as part of a work-related or medical procedure, e.g., administered as a prophylactic measure. In another embodiment, the radiation or toxin exposure is received unintentionally. In such a case, the compound is preferably administered as soon as possible after the exposure to inhibit apoptosis and the subsequent development of acute radiation syndrome.

Sirtuin-modulating compounds may also be used for treating and/or preventing cancer. In certain embodiments, sirtuin-modulating compounds that increase the level and/or activity of a sirtuin protein may be used for treating and/or preventing cancer. Calorie restriction has been linked to a reduction in the incidence of age-related disorders including cancer. Accordingly, an increase in the level and/or activity of a sirtuin protein may be useful for treating and/or preventing the incidence of age-related disorders, such as, for example, cancer. Exemplary cancers that may be treated using a sirtuin-modulating compound are those of the brain and kidney; hormone-dependent cancers including breast, prostate, testicular, and ovarian cancers; lymphomas, and leukemias. In cancers associated with solid tumors, a modulating compound may be administered directly into the tumor. Cancer of blood cells, e.g., leukemia, can be treated by administering a modulating compound into the blood stream or into the bone marrow. Benign cell growth, e.g., warts, can also be treated. Other diseases that can be treated include autoimmune diseases, e.g., systemic lupus erythematosus, scleroderma, and arthritis, in which autoimmune cells should be removed. Viral infections such as herpes, HIV, adenovirus, and HTLV-1 associated malignant and benign disorders can also be treated by administration of sirtuin-modulating compound. Alternatively, cells can be obtained from a subject, treated ex vivo to remove certain undesirable cells, e.g., cancer cells, and administered back to the same or a different subject.

Chemotherapeutic agents may be co-administered with modulating compounds described herein as having anti-cancer activity, e.g., compounds that induce apoptosis, compounds that reduce lifespan or compounds that render cells sensitive to stress. Chemotherapeutic agents may be used by themselves with a sirtuin-modulating compound described herein as inducing cell death or reducing lifespan or increasing sensitivity to stress and/or in combination with other chemotherapeutics agents. In addition to conventional chemotherapeutics, the sirtuin-modulating compounds described herein may also be used with antisense RNA, RNAi or other polynucleotides to inhibit the expression of the cellular components that contribute to unwanted cellular proliferation.

Combination therapies comprising sirtuin-modulating compounds and a conventional chemotherapeutic agent may be advantageous over combination therapies known in the art because the combination allows the conventional chemotherapeutic agent to exert greater effect at lower dosage. In a preferred embodiment, the effective dose (ED₅₀) for a chemotherapeutic agent, or combination of conventional chemotherapeutic agents, when used in combination with a sirtuin-modulating compound is at least 2 fold less than the ED₅₀ for the chemotherapeutic agent alone, and even more preferably at 5 fold, 10 fold or even 25 fold less. Conversely, the therapeutic index (TI) for such chemotherapeutic agent or combination of such chemotherapeutic agent when used in combination with a sirtuin-modulating compound described herein can be at least 2 fold greater than the TI for conventional chemotherapeutic regimen alone, and even more preferably at 5 fold, 10 fold or even 25 fold greater.

### Neuronal Diseases/Disorders

In certain aspects, sirtuin-modulating compounds that increase the level and/or activity of a sirtuin protein can be used to treat patients suffering from neurodegenerative diseases, and traumatic or mechanical injury to the central nervous system (CNS), spinal cord or peripheral nervous system (PNS), wherein the subject has a T373N or L107X Sirt1 polymorphic variant. Neurodegenerative disease typically involves reductions in the mass and volume of the human brain, which may be due to the atrophy and/or death of brain cells, which are far more profound than those in a healthy person that are attributable to aging. Neurodegenerative diseases can evolve gradually, after a long period of normal brain function, due to progressive degeneration (e.g., nerve cell dysfunction and death) of specific brain regions. Alternatively, neurodegenerative diseases can have a quick onset, such as those associated with trauma or toxins. The actual onset of brain degeneration may precede clinical expression by many years. Examples of neurodegenerative diseases include, but are not limited to, Alzheimer's disease (AD), Parkinson's disease (PD), Huntington's disease (HD), amyotrophic lateral sclerosis (ALS; Lou Gehrig's disease), diffuse Lewy body disease, chorea-acanthocytosis, primary lateral sclerosis, ocular diseases (ocular neuritis), chemotherapy-induced neuropathies (e.g., from vincristine, paclitaxel, bortezomib), diabetes-induced neuropathies and Friedreich's ataxia. Sirtuin-modulating compounds that increase the level and/or activity of a sirtuin protein can be used to treat these disorders and others as described below.

AD is a CNS disorder that results in memory loss, unusual behavior, personality changes, and a decline in thinking abilities. These losses are related to the death of specific types of brain cells and the breakdown of connections and their supporting network (e.g. glial cells) between them. The earliest symptoms include loss of recent memory, faulty judgment, and changes in personality. PD is a CNS disorder that results in uncontrolled body movements, rigidity, tremor, and dyskinesia, and is associated with the death of brain cells in an area of the brain that produces dopamine. ALS (motor neuron disease) is a CNS disorder that attacks the motor neurons, components of the CNS that connect the brain to the skeletal muscles.

HD is another neurodegenerative disease that causes uncontrolled movements, loss of intellectual faculties, and emotional disturbance. Tay-Sachs disease and Sandhoff disease are glycolipid storage diseases where GM2 ganglioside and related glycolipid substrates for β-hexosaminidase accumulate in the nervous system and trigger acute neurodegeneration.

It is well-known that apoptosis plays a role in AIDS pathogenesis in the immune system. However, HIV-1 also induces neurological disease, which can be treated with sirtuin-modulating compounds of the invention.

Neuronal loss is also a salient feature of prion diseases, such as Creutzfeldt-Jakob disease in human, BSE in cattle (mad cow disease), Scrapie Disease in sheep and goats, and feline spongiform encephalopathy (FSE) in cats. Sirtuin-modulating compounds that increase the level and/or activity of a sirtuin protein may be useful for treating or preventing neuronal loss due to these prior diseases.

In another embodiment, a sirtuin-modulating compound that increases the level and/or activity of a sirtuin protein may be used to treat or prevent any disease or disorder involving axonopathy. Distal axonopathy is a type of peripheral neuropathy that results from some metabolic or toxic derangement of peripheral nervous system (PNS) neurons. It is the most common response of nerves to metabolic or toxic disturbances, and as such may be caused by metabolic diseases such as diabetes, renal failure, deficiency syndromes such as malnutrition and alcoholism, or the effects of toxins or drugs. Those with distal axonopathies usually present with symmetrical glove-stocking sensory-motor disturbances. Deep tendon reflexes and autonomic nervous system (ANS) functions are also lost or diminished in affected areas.

Diabetic neuropathies are neuropathic disorders that are associated with diabetes mellitus. Relatively common conditions which may be associated with diabetic neuropathy include third nerve palsy; mononeuropathy; mononeuritis multiplex; diabetic amyotrophy; a painful polyneuropathy; autonomic neuropathy; and thoracoabdominal neuropathy.

Peripheral neuropathy is the medical term for damage to nerves of the peripheral nervous system, which may be caused either by diseases of the nerve or from the side-effects of systemic illness. Major causes of peripheral neuropathy include seizures, nutritional deficiencies, and HIV, though diabetes is the most likely cause.

In an exemplary embodiment, a sirtuin-modulating compound that increases the level and/or activity of a sirtuin protein may be used to treat or prevent multiple sclerosis (MS), including relapsing MS and monosymptomatic MS, and other demyelinating conditions, such as, for example, chromic inflammatory demyelinating polyneuropathy (CIDP), or symptoms associated therewith.

In yet another embodiment, a sirtuin-modulating compound that increases the level and/or activity of a sirtuin protein may be used to treat trauma to the nerves, including, trauma due to disease, injury (including surgical intervention), or environmental trauma (e.g., neurotoxins, alcoholism, etc.).

Sirtuin-modulating compounds that increase the level and/or activity of a sirtuin protein may also be useful to prevent, treat, and alleviate symptoms of various PNS disorders. The term “peripheral neuropathy” encompasses a wide range of disorders in which the nerves outside of the brain and spinal cord—peripheral nerves—have been damaged. Peripheral neuropathy may also be referred to as peripheral neuritis, or if many nerves are involved, the terms polyneuropathy or polyneuritis may be used.

PNS diseases treatable with sirtuin-modulating compounds that increase the level and/or activity of a sirtuin protein include: diabetes, leprosy, Charcot-Marie-Tooth disease, Guillain-Barré syndrome and Brachial Plexus Neuropathies (diseases of the cervical and first thoracic roots, nerve trunks, cords, and peripheral nerve components of the brachial plexus.

In another embodiment, a sirtuin activating compound may be used to treat or prevent a polyglutamine disease. Exemplary polyglutamine diseases include Spinobulbar muscular atrophy (Kennedy disease), Huntington's Disease (HD), Dentatorubral-pallidoluysian atrophy (Haw River syndrome), Spinocerebellar ataxia type 1, Spinocerebellar ataxia type 2, Spinocerebellar ataxia type 3 (Machado-Joseph disease), Spinocerebellar ataxia type 6, Spinocerebellar ataxia type 7, and Spinocerebellar ataxia type 17.

In certain embodiments, the invention provides a method to treat a central nervous system cell to prevent damage in response to a decrease in blood flow to the cell. Typically the severity of damage that may be prevented will depend in large part on the degree of reduction in blood flow to the cell and the duration of the reduction. In one embodiment, apoptotic or necrotic cell death may be prevented. In still a further embodiment, ischemic-mediated damage, such as cytoxic edema or central nervous system tissue anoxemia, may be prevented. In each embodiment, the central nervous system cell may be a spinal cell or a brain cell.

Another aspect encompasses administrating a sirtuin activating compound to a subject to treat a central nervous system ischemic condition. A number of central nervous system ischemic conditions may be treated by the sirtuin activating compounds described herein. In one embodiment, the ischemic condition is a stroke that results in any type of ischemic central nervous system damage, such as apoptotic or necrotic cell death, cytoxic edema or central nervous system tissue anoxia. The stroke may impact any area of the brain or be caused by any etiology commonly known to result in the occurrence of a stroke. In one alternative of this embodiment, the stroke is a brain stem stroke. In another alternative of this embodiment, the stroke is a cerebellar stroke. In still another embodiment, the stroke is an embolic stroke. In yet another alternative, the stroke may be a hemorrhagic stroke. In a further embodiment, the stroke is a thrombotic stroke.

In yet another aspect, a sirtuin activating compound may be administered to reduce infarct size of the ischemic core following a central nervous system ischemic condition. Moreover, a sirtuin activating compound may also be beneficially administered to reduce the size of the ischemic penumbra or transitional zone following a central nervous system ischemic condition.

In one embodiment, a combination drug regimen may include drugs or compounds for the treatment or prevention of neurodegenerative disorders or secondary conditions associated with these conditions. Thus, a combination drug regimen may include one or more sirtuin activators and one or more anti-neurodegeneration agents.

### Blood Coagulation Disorders

In other aspects, sirtuin-modulating compounds that increase the level and/or activity of a sirtuin protein can be used to treat or prevent blood coagulation disorders (or hemostatic disorders) in a subject having a T373N or L107X Sirt1 polymorphic variant. As used interchangeably herein, the terms “hemostasis”, “blood coagulation,” and “blood clotting” refer to the control of bleeding, including the physiological properties of vasoconstriction and coagulation. Blood coagulation assists in maintaining the integrity of mammalian circulation after injury, inflammation, disease, congenital defect, dysfunction or other disruption. Further, the formation of blood clots does not only limit bleeding in case of an injury (hemostasis), but may lead to serious organ damage and death in the context of atherosclerotic diseases by occlusion of an important artery or vein. Thrombosis is thus blood clot formation at the wrong time and place.

Accordingly, the present invention provides anticoagulation and antithrombotic treatments aiming at inhibiting the formation of blood clots in order to prevent or treat blood coagulation disorders, such as myocardial infarction, stroke, loss of a limb by peripheral artery disease or pulmonary embolism.

As used interchangeably herein, “modulating or modulation of hemostasis” and “regulating or regulation of hemostasis” includes the induction (e.g., stimulation or increase) of hemostasis, as well as the inhibition (e.g., reduction or decrease) of hemostasis.

In one aspect, the invention provides a method for reducing or inhibiting hemostasis in a subject by administering a sirtuin-modulating compound that increases the level and/or activity of a sirtuin protein. The compositions and methods disclosed herein are useful for the treatment or prevention of thrombotic disorders. As used herein, the term “thrombotic disorder” includes any disorder or condition characterized by excessive or unwanted coagulation or hemostatic activity, or a hypercoagulable state. Thrombotic disorders include diseases or disorders involving platelet adhesion and thrombus formation, and may manifest as an increased propensity to form thromboses, e.g., an increased number of thromboses, thrombosis at an early age, a familial tendency towards thrombosis, and thrombosis at unusual sites.

In another embodiment, a combination drug regimen may include drugs or compounds for the treatment or prevention of blood coagulation disorders or secondary conditions associated with these conditions. Thus, a combination drug regimen may include one or more sirtuin-modulating compounds that increase the level and/or activity of a sirtuin protein and one or more anti-coagulation or anti-thrombosis agents.

### Weight Control

In another aspect, sirtuin-modulating compounds that increase the level and/or activity of a sirtuin protein may be used for treating or preventing weight gain or obesity in a subject having a T373N or L107X Sirt1 polymorphic variant. For example, sirtuin-modulating compounds that increase the level and/or activity of a sirtuin protein may be used, for example, to treat or prevent hereditary obesity, dietary obesity, hormone related obesity, obesity related to the administration of medication, to reduce the weight of a subject, or to reduce or prevent weight gain in a subject. A subject in need of such a treatment may be a subject who is obese, likely to become obese, overweight, or likely to become overweight. Subjects who are likely to become obese or overweight can be identified, for example, based on family history, genetics, diet, activity level, medication intake, or various combinations thereof.

In yet other embodiments, sirtuin-modulating compounds that increase the level and/or activity of a sirtuin protein may be administered to subjects suffering from a variety of other diseases and conditions that may be treated or prevented by promoting weight loss in the subject. Such diseases include, for example, high blood pressure, hypertension, high blood cholesterol, dyslipidemia, type 2 diabetes, insulin resistance, glucose intolerance, hyperinsulinemia, coronary heart disease, angina pectoris, congestive heart failure, stroke, gallstones, cholescystitis and cholelithiasis, gout, osteoarthritis, obstructive sleep apnea and respiratory problems, some types of cancer (such as endometrial, breast, prostate, and colon), complications of pregnancy, poor female reproductive health (such as menstrual irregularities, infertility, irregular ovulation), bladder control problems (such as stress incontinence); uric acid nephrolithiasis; psychological disorders (such as depression, eating disorders, distorted body image, and low self esteem). Finally, patients with AIDS can develop lipodystrophy or insulin resistance in response to combination therapies for AIDS.

In another embodiment, sirtuin-modulating compounds that increase the level and/or activity of a sirtuin protein may be used for inhibiting adipogenesis or fat cell differentiation, whether in vitro or in vivo. Such methods may be used for treating or preventing obesity.

In other embodiments, sirtuin-modulating compounds that increase the level and/or activity of a sirtuin protein may be used for reducing appetite and/or increasing satiety, thereby causing weight loss or avoidance of weight gain. A subject in need of such a treatment may be a subject who is overweight, obese or a subject likely to become overweight or obese. The method may comprise administering daily or, every other day, or once a week, a dose, e.g., in the form of a pill, to a subject. The dose may be an “appetite reducing dose.”

In an exemplary embodiment, sirtuin-modulating compounds that increase the level and/or activity of a sirtuin protein may be administered as a combination therapy for treating or preventing weight gain or obesity. For example, one or more sirtuin-modulating compounds that increase the level and/or activity of a sirtuin protein may be administered in combination with one or more anti-obesity agents.

In another embodiment, sirtuin-modulating compounds that increase the level and/or activity of a sirtuin protein may be administered to reduce drug-induced weight gain. For example, a sirtuin-modulating compound that increases the level and/or activity of a sirtuin protein may be administered as a combination therapy with medications that may stimulate appetite or cause weight gain, in particular, weight gain due to factors other than water retention.

### Metabolic Disorders/Diabetes

In another aspect, sirtuin-modulating compounds that increase the level and/or activity of a sirtuin protein may be used for treating or preventing a metabolic disorder, such as insulin-resistance, an insulin-resistance disorder, a pre-diabetic state, type 1 diabetes, type 2 diabetes, type 1.5 diabetes, and/or complications thereof in a subject having a T373N or L107X Sirt1 polymorphic variant. Administration of a sirtuin-modulating compound that increases the level and/or activity of a sirtuin protein may increase insulin sensitivity and/or decrease insulin levels in a subject. A subject in need of such a treatment may be a subject who has insulin resistance or other precursor symptom of type II diabetes, who has type II diabetes, or who is likely to develop any of these conditions. For example, the subject may be a subject having insulin resistance, e.g., having high circulating levels of insulin and/or associated conditions, such as hyperlipidemia, dyslipogenesis, hypercholesterolemia, impaired glucose tolerance, high blood glucose sugar level, other manifestations of syndrome X, hypertension, atherosclerosis and lipodystrophy.

In an exemplary embodiment, sirtuin-modulating compounds that increase the level and/or activity of a sirtuin protein may be administered as a combination therapy for treating or preventing a metabolic disorder. For example, one or more sirtuin-modulating compounds that increase the level and/or activity of a sirtuin protein may be administered in combination with one or more anti-diabetic agents.

“Diabetes” refers to high blood sugar or ketoacidosis, as well as chronic, general metabolic abnormalities arising from a prolonged high blood sugar status or a decrease in glucose tolerance. “Diabetes” encompasses both the type I and type II (Non Insulin Dependent Diabetes Mellitus or NIDDM) forms of the disease. The risk factors for diabetes include the following factors: waistline of more than 40 inches for men or 35 inches for women, blood pressure of 130/85 mmHg or higher, triglycerides above 150 mg/dl, fasting blood glucose greater than 100 mg/dl or high-density lipoprotein of less than 40 mg/dl in men or 50 mg/dl in women.

The term “hyperinsulinemia” refers to a state in an individual in which the level of insulin in the blood is higher than normal.

The term “insulin resistance” refers to a state in which a normal amount of insulin produces a subnormal biologic response relative to the biological response in a subject that does not have insulin resistance.

An “insulin resistance disorder,” as discussed herein, refers to any disease or condition that is caused by or contributed to by insulin resistance. Examples include: diabetes, obesity, metabolic syndrome, insulin-resistance syndromes, syndrome X, insulin resistance, high blood pressure, hypertension, high blood cholesterol, dyslipidemia, hyperlipidemia, dyslipidemia, atherosclerotic disease including stroke, coronary artery disease or myocardial infarction, hyperglycemia, hyperinsulinemia and/or hyperproinsulinemia, impaired glucose tolerance, delayed insulin release, diabetic complications, including coronary heart disease, angina pectoris, congestive heart failure, stroke, cognitive functions in dementia, retinopathy, peripheral neuropathy, nephropathy, glomerulonephritis, glomerulosclerosis, nephrotic syndrome, hypertensive nephrosclerosis some types of cancer (such as endometrial, breast, prostate, and colon), complications of pregnancy, poor female reproductive health (such as menstrual irregularities, infertility, irregular ovulation, polycystic ovarian syndrome (PCOS)), lipodystrophy, cholesterol related disorders, such as gallstones, cholescystitis and cholelithiasis, gout, obstructive sleep apnea and respiratory problems, osteoarthritis, and prevention and treatment of bone loss, e.g. osteoporosis.

Metabolic syndrome indicates an elevated risk of developing diabetes. Metabolic syndrome may be diagnosed using a number of tests including a fasting glucose test, glucose tolerance test, lipid profile test, sdLDL test, fasting insulin test, microalbumin test, hs-CRP test, blood pressure test, or test for obesity. A glucose test may be a fasting glucose test or a postprandial glucose test. In performing a lipid profile test, one may measure levels of one or more of HDL, LDL, triglycerides, VLDL and DLDL (direct measurement of the LDL). Microalbumin tests can identify kidney dysfunction, a pathology often associated with metabolic disease. An hs-CRP test identifies inflammation, and helps to predict the risk of cardiac complications in a patient with metabolic syndrome. An sdLDL test determines the level of small dense low-density lipoprotein molecules in a patient; sdLDL comprises a fraction of the total LDL content of a patient's serum. sdLDL may be a better indicator than total LDL in predicting atherosclerosis, another complication of metabolic syndrome.

In certain embodiments, methods for determining a subject's risk for developing diabetes or methods for treating diabetes may involve determining the presence of a T373N or L107P Sirt1 polymorphic variant in conjunction with other genetic tests that are predictive of a predisposition to diabetes. For example, such a genetic test may comprise determining the nucleotide or amino acid sequence of a DQ HLA allele, L-selectin, PPAR gamma, hepatocyte nuclear factor 1-a, HNF4-a, Insulin receptor substrate-1, Insulin receptor substrate-2, PGC-1 alpha, KCNJI1, ABCC8, GLUT1, GLUT4, calpain 10, glucagon receptor, human beta 3 adrenergic receptor, fatty acid binding protein 2, mitochondrial tRNA [Leu (UUR)], sulphonylurea receptor, UCP2, UCP3, PTPN1, adiponectin, TCF7L2, or amylin, or the regulatory nucleotide sequences thereof.

Examples of genetic polymorphisms that affect predisposition to diabetes are as follows. A P12A polymorphism of the PPAR gamma gene protects against diabetes. The Ala98Val polymorphism of HNF1-a causes predisposition to type 1 diabetes. Plasma cell glycoprotein (PC-1) K121Q is associated with a higher risk of diabetes in at least some populations. Three polymorphisms of PGC-1 alpha, Thr394Thr, Gly482Ser and +A2962G correlate with type 2 diabetes. Also, the D1057 D variant of 1RS-2 predisposes a carrier to diabetes. GLUT 1 and GLUT 4 may also be tested, because glucokinase influences diabetes, and a polymorphism in GLUT1 correlates with a susceptibility to type 2 diabetes. An intronic variant (UCSNP-43: G to A) and a haplotype combination (UCSNP-43, -19, and -63) of calpain 10 correlate with development of type 2 diabetes. The T668C nucleotide mutation of L-selectin causes a F206L change in the protein, which influences diabetes susceptibility. Mutations in APM1 at nucleotide 45 (G allele) in exon 2 and 276 in intron 2 (T allele) have been linked to impaired glucose tolerance. In the amylin gene, m215T>G and m132G>A mutations are present at elevated levels in type 2 diabetes patients, suggesting that these alleles promote the development of diabetes.

In certain embodiments, the methods disclosed herein comprise administering to a subject having a T373N or L107P Sirt1 polymorphic variant an anti-diabetic agent, a sirtuin activating compound, or combinations thereof. Exemplary anti-diabetic agents include the following: an aldose reductase inhibitor, a glycogen phosphorylase inhibitor, a sorbitol dehydrogenase inhibitor, a protein tyrosine phosphatase 1B inhibitor, a dipeptidyl protease inhibitor, insulin (including orally bioavailable insulin preparations), an insulin mimetic, metformin, acarbose, a peroxisome proliferator-activated receptor-gamma (PPAR-gamma) ligand such as troglitazone, rosaglitazone, pioglitazone or GW-1929, a sulfonylurea, glipazide, glyburide, or chlorpropamide wherein the amounts of the first and second compounds result in a therapeutic effect. Other anti-diabetic agents include a glucosidase inhibitor, a glucagon-like peptide-1 (GLP-1), a PPAR alpha/gamma dual agonist, a meglitimide and an alpha-P2 inhibitor. In an exemplary embodiment, an anti-diabetic agent may be metformin, a dipeptidyl peptidase IV (DP-IV or DPP-IV) inhibitor, such as, for example LAF237 from Novartis (NVP DPP728; 1-[[[2-[(5-cyanopyridin-2-yl)amino]ethyl]amino]acetyl]-2-cyano-(S)-pyrrol-idine) or MK-04301 from Merck (see e.g., Hughes et al., Biochemistry 38: 11597-603 (1999)).

### Inflammatory Diseases

In other aspects, sirtuin-modulating compounds that increase the level and/or activity of a sirtuin protein can be used to treat or prevent a disease or disorder associated with inflammation, in a subject having a T373N or L107X Sirt1 polymorphic variant. Sirtuin-modulating compounds that increase the level and/or activity of a sirtuin protein may be administered prior to the onset of, at, or after the initiation of inflammation. When used prophylactically, the compounds are preferably provided in advance of any inflammatory response or symptom. Administration of the compounds may prevent or attenuate inflammatory responses or symptoms.

In another embodiment, sirtuin-modulating compounds that increase the level and/or activity of a sirtuin protein may be used to treat or prevent allergies and respiratory conditions, including asthma, bronchitis, pulmonary fibrosis, allergic rhinitis, oxygen toxicity, emphysema, chronic bronchitis, acute respiratory distress syndrome, and any chronic obstructive pulmonary disease (COPD). The compounds may be used to treat chronic hepatitis infection, including hepatitis B and hepatitis C.

Additionally, sirtuin-modulating compounds that increase the level and/or activity of a sirtuin protein may be used to treat autoimmune diseases and/or inflammation associated with autoimmune diseases such as organ-tissue autoimmune diseases (e.g., Raynaud's syndrome), scleroderma, myasthenia gravis, transplant rejection, endotoxin shock, sepsis, psoriasis, eczema, dermatitis, multiple sclerosis, autoimmune thyroiditis, uveitis, systemic lupus erythematosis, Addison's disease, autoimmune polyglandular disease (also known as autoimmune polyglandular syndrome), and Grave's disease.

In certain embodiments, one or more sirtuin-modulating compounds that increase the level and/or activity of a sirtuin protein may be taken alone or in combination with other compounds useful for treating or preventing inflammation.

### Flushing

In another aspect, sirtuin-modulating compounds that increase the level and/or activity of a sirtuin protein may be used for reducing the incidence or severity of flushing and/or hot flashes which are symptoms of a disorder in a subject having a T373N or L107X Sirt1 polymorphic variant. For instance, the subject method includes the use of sirtuin-modulating compounds that increase the level and/or activity of a sirtuin protein, alone or in combination with other agents, for reducing incidence or severity of flushing and/or hot flashes in cancer patients. In other embodiments, the method provides for the use of sirtuin-modulating compounds that increase the level and/or activity of a sirtuin protein to reduce the incidence or severity of flushing and/or hot flashes in menopausal and post-menopausal woman.

In another aspect, sirtuin-modulating compounds that increase the level and/or activity of a sirtuin protein may be used as a therapy for reducing the incidence or severity of flushing and/or hot flashes which are side-effects of another drug therapy, e.g., drug-induced flushing. In certain embodiments, a method for treating and/or preventing drug-induced flushing comprises administering to a patient in need thereof a formulation comprising at least one flushing inducing compound and at least one sirtuin-modulating compound that increases the level and/or activity of a sirtuin protein. In other embodiments, a method for treating drug induced flushing comprises separately administering one or more compounds that induce flushing and one or more sirtuin-modulating compounds, e.g., wherein the sirtuin-modulating compound and flushing inducing agent have not been formulated in the same compositions. When using separate formulations, the sirtuin-modulating compound may be administered (1) at the same as administration of the flushing inducing agent, (2) intermittently with the flushing inducing agent, (3) staggered relative to administration of the flushing inducing agent, (4) prior to administration of the flushing inducing agent, (5) subsequent to administration of the flushing inducing agent, and (6) various combination thereof. Exemplary flushing inducing agents include, for example, niacin, faloxifene, antidepressants, anti-psychotics, chemotherapeutics, calcium channel blockers, and antibiotics.

In one embodiment, sirtuin-modulating compounds that increase the level and/or activity of a sirtuin protein may be used to reduce flushing side effects of a vasodilator or an antilipemic agent (including anticholesteremic agents and lipotropic agents). In an exemplary embodiment, a sirtuin-modulating compound that increases the level and/or activity of a sirtuin protein may be used to reduce flushing associated with the administration of niacin.

In another embodiment, the invention provides a method for treating and/or preventing hyperlipidemia with reduced flushing side effects. In another representative embodiment, the method involves the use of sirtuin-modulating compounds that increase the level and/or activity of a sirtuin protein to reduce flushing side effects of raloxifene. In another representative embodiment, the method involves the use of sirtuin-modulating compounds that increase the level and/or activity of a sirtuin protein to reduce flushing side effects of antidepressants or anti-psychotic agent. For instance, sirtuin-modulating compounds that increase the level and/or activity of a sirtuin protein can be used in conjunction (administered separately or together) with a serotonin reuptake inhibitor, or a 5HT2 receptor antagonist.

In certain embodiments, sirtuin-modulating compounds that increase the level and/or activity of a sirtuin protein may be used as part of a treatment with a serotonin reuptake inhibitor (SRI) to reduce flushing. In still another representative embodiment, sirtuin-modulating compounds that increase the level and/or activity of a sirtuin protein may be used to reduce flushing side effects of chemotherapeutic agents, such as cyclophosphamide and tamoxifen.

In another embodiment, sirtuin-modulating compounds that increase the level and/or activity of a sirtuin protein may be used to reduce flushing side effects of calcium channel blockers, such as amlodipine.

In another embodiment, sirtuin-modulating compounds that increase the level and/or activity of a sirtuin protein may be used to reduce flushing side effects of antibiotics. For example, sirtuin-modulating compounds that increase the level and/or activity of a sirtuin protein can be used in combination with levofloxacin.

### Ocular Disorders

One aspect of the present invention is a method for inhibiting, reducing or otherwise treating vision impairment by administering to a subject a therapeutic dosage of sirtuin modulator, wherein the subject has a T373N or L107X Sirt1 polymorphic variant.

In certain aspects of the invention, the vision impairment is caused by damage to the optic nerve or central nervous system. In particular embodiments, optic nerve damage is caused by high intraocular pressure, such as that created by glaucoma. In other particular embodiments, optic nerve damage is caused by swelling of the nerve, which is often associated with an infection or an immune (e.g., autoimmune) response such as in optic neuritis.

In certain aspects of the invention, the vision impairment is caused by retinal damage. In particular embodiments, retinal damage is caused by disturbances in blood flow to the eye (e.g., arteriosclerosis, vasculitis). In particular embodiments, retinal damage is caused by disruption of the macula (e.g., exudative or non-exudative macular degeneration).

Exemplary retinal diseases include Exudative Age Related Macular Degeneration, Nonexudative Age Related Macular Degeneration, Retinal Electronic Prosthesis and RPE Transplantation Age Related Macular Degeneration, Acute Multifocal Placoid Pigment Epitheliopathy, Acute Retinal Necrosis, Best Disease, Branch Retinal Artery Occlusion, Branch Retinal Vein Occlusion, Cancer Associated and Related Autoimmune Retinopathies, Central Retinal Artery Occlusion, Central Retinal Vein Occlusion, Central Serous Chorioretinopathy, Eales Disease, Epimacular Membrane, Lattice Degeneration, Macroaneurysm, Diabetic Macular Edema, Irvine-Gass Macular Edema, Macular Hole, Subretinal Neovascular Membranes, Diffuse Unilateral Subacute Neuroretinitis, Nonpseudophakic Cystoid Macular Edema, Presumed Ocular Histoplasmosis Syndrome, Exudative Retinal Detachment, Postoperative Retinal Detachment, Proliferative Retinal Detachment, Rhegmatogenous Retinal Detachment, Tractional Retinal Detachment, Retinitis Pigmentosa, CMV Retinitis, Retinoblastoma, Retinopathy of Prematurity, Birdshot Retinopathy, Background Diabetic Retinopathy, Proliferative Diabetic Retinopathy, Hemoglobinopathies Retinopathy, Purtscher Retinopathy, Valsalva Retinopathy, Juvenile Retinoschisis, Senile Retinoschisis, Terson Syndrome and White Dot Syndromes.

Other exemplary diseases include ocular bacterial infections (e.g. conjunctivitis, keratitis, tuberculosis, syphilis, gonorrhea), viral infections (e.g. Ocular Herpes Simplex Virus, Varicella Zoster Virus, Cytomegalovirus retinitis, Human Immunodeficiency Virus (HIV)) as well as progressive outer retinal necrosis secondary to HIV or other HIV-associated and other immunodeficiency-associated ocular diseases. In addition, ocular diseases include fungal infections (e.g. Candida choroiditis, histoplasmosis), protozoal infections (e.g. toxoplasmosis) and others such as ocular toxocariasis and sarcoidosis.

One aspect of the invention is a method for inhibiting, reducing or treating vision impairment in a subject undergoing treatment with a chemotherapeutic drug (e.g., a neurotoxic drug, a drug that raises intraocular pressure such as a steroid), by administering to the subject in need of such treatment a therapeutic dosage of a sirtuin modulator disclosed herein.

Another aspect of the invention is a method for inhibiting, reducing or treating vision impairment in a subject undergoing surgery, including ocular or other surgeries performed in the prone position such as spinal cord surgery, by administering to the subject in need of such treatment a therapeutic dosage of a sirtuin modulator disclosed herein. Ocular surgeries include cataract, iridotomy and lens replacements.

Another aspect of the invention is the treatment, including inhibition and prophylactic treatment, of age related ocular diseases include cataracts, dry eye, age-related macular degeneration (AMD), retinal damage and the like, by administering to the subject in need of such treatment a therapeutic dosage of a sirtuin modulator disclosed herein.

Another aspect of the invention is the prevention or treatment of damage to the eye caused by stress, chemical insult or radiation, by administering to the subject in need of such treatment a therapeutic dosage of a sirtuin modulator disclosed herein. Radiation or electromagnetic damage to the eye can include that caused by CRT's or exposure to sunlight or UV.

In one embodiment, a combination drug regimen may include drugs or compounds for the treatment or prevention of ocular disorders or secondary conditions associated with these conditions. Thus, a combination drug regimen may include one or more sirtuin activators and one or more therapeutic agents for the treatment of an ocular disorder.

In one embodiment, a sirtuin modulator can be administered in conjunction with a therapy for reducing intraocular pressure. In another embodiment, a sirtuin modulator can be administered in conjunction with a therapy for treating and/or preventing glaucoma. In yet another embodiment, a sirtuin modulator can be administered in conjunction with a therapy for treating and/or preventing optic neuritis. In one embodiment, a sirtuin modulator can be administered in conjunction with a therapy for treating and/or preventing CMV Retinopathy. In another embodiment, a sirtuin modulator can be administered in conjunction with a therapy for treating and/or preventing multiple sclerosis.

### Mitochondrial-Associated Diseases and Disorders

In certain embodiments, the invention provides methods for treating diseases or disorders that would benefit from increased mitochondrial activity in subject having a T373N or L107X Sirt1 polymorphic variant. The methods involve administering to a subject in need thereof a therapeutically effective amount of a sirtuin activating compound. Increased mitochondrial activity refers to increasing activity of the mitochondria while maintaining the overall numbers of mitochondria (e.g., mitochondrial mass), increasing the numbers of mitochondria thereby increasing mitochondrial activity (e.g., by stimulating mitochondrial biogenesis), or combinations thereof. In certain embodiments, diseases and disorders that would benefit from increased mitochondrial activity include diseases or disorders associated with mitochondrial dysfunction.

In certain embodiments, methods for treating diseases or disorders that would benefit from increased mitochondrial activity may comprise identifying a subject suffering from a mitochondrial dysfunction. Methods for diagnosing a mitochondrial dysfunction may involve molecular genetic, pathologic and/or biochemical analyses. Diseases and disorders associated with mitochondrial dysfunction include diseases and disorders in which deficits in mitochondrial respiratory chain activity contribute to the development of pathophysiology of such diseases or disorders in a mammal. Diseases or disorders that would benefit from increased mitochondrial activity generally include for example, diseases in which free radical mediated oxidative injury leads to tissue degeneration, diseases in which cells inappropriately undergo apoptosis, and diseases in which cells fail to undergo apoptosis.

In certain embodiments, the invention provides methods for treating a disease or disorder that would benefit from increased mitochondrial activity that involves administering to a subject in need thereof one or more sirtuin activating compounds in combination with another therapeutic agent such as, for example, an agent useful for treating mitochondrial dysfunction or an agent useful for reducing a symptom associated with a disease or disorder involving mitochondrial dysfunction.

In exemplary embodiments, the invention provides methods for treating diseases or disorders that would benefit from increased mitochondrial activity by administering to a subject a therapeutically effective amount of a sirtuin activating compound. Exemplary diseases or disorders include, for example, neuromuscular disorders (e.g., Friedreich's Ataxia, muscular dystrophy, multiple sclerosis, etc.), disorders of neuronal instability (e.g., seizure disorders, migraine, etc.), developmental delay, neurodegenerative disorders (e.g., Alzheimer's Disease, Parkinson's Disease, amyotrophic lateral sclerosis, etc.), ischemia, renal tubular acidosis, age-related neurodegeneration and cognitive decline, chemotherapy fatigue, age-related or chemotherapy-induced menopause or irregularities of menstrual cycling or ovulation, mitochondrial myopathies, mitochondrial damage (e.g., calcium accumulation, excitotoxicity, nitric oxide exposure, hypoxia, etc.), and mitochondrial deregulation.

Muscular dystrophy refers to a family of diseases involving deterioration of neuromuscular structure and function, often resulting in atrophy of skeletal muscle and myocardial dysfunction, such as Duchenne muscular dystrophy. In certain embodiments, sirtuin activating compounds may be used for reducing the rate of decline in muscular functional capacities and for improving muscular functional status in patients with muscular dystrophy.

In certain embodiments, sirtuin modulating compounds may be useful for treatment mitochondrial myopathies. Mitochondrial myopathies range from mild, slowly progressive weakness of the extraocular muscles to severe, fatal infantile myopathies and multisystem encephalomyopathies. Some syndromes have been defined, with some overlap between them. Established syndromes affecting muscle include progressive external ophthalmoplegia, the Kearns-Sayre syndrome (with ophthalmoplegia, pigmentary retinopathy, cardiac conduction defects, cerebellar ataxia, and sensorineural deafness), the MELAS syndrome (mitochondrial encephalomyopathy, lactic acidosis, and stroke-like episodes), the MERFF syndrome (myoclonic epilepsy and ragged red fibers), limb-girdle distribution weakness, and infantile myopathy (benign or severe and fatal).

In certain embodiments, sirtuin activating compounds may be useful for treating patients suffering from toxic damage to mitochondria, such as, toxic damage due to calcium accumulation, excitotoxicity, nitric oxide exposure, drug induced toxic damage, or hypoxia.

In certain embodiments, sirtuin activating compounds may be useful for treating diseases or disorders associated with mitochondrial deregulation.

### Muscle Performance

In other embodiments, the invention provides methods for enhancing muscle performance in a subject, by administering a therapeutically effective amount of a sirtuin activating compound to a subject having a T373N or L107X Sirt1 polymorphic variant. For example, sirtuin activating compounds may be useful for improving physical endurance (e.g., ability to perform a physical task such as exercise, physical labor, sports activities, etc), inhibiting or retarding physical fatigues, enhancing blood oxygen levels, enhancing energy in healthy individuals, enhance working capacity and endurance, reducing muscle fatigue, reducing stress, enhancing cardiac and cardiovascular function, improving sexual ability, increasing muscle ATP levels, and/or reducing lactic acid in blood. In certain embodiments, the methods involve administering an amount of a sirtuin activating compound that increase mitochondrial activity, increase mitochondrial biogenesis, and/or increase mitochondrial mass.

Sports performance refers to the ability of the athlete's muscles to perform when participating in sports activities. Enhanced sports performance, strength, speed and endurance are measured by an increase in muscular contraction strength, increase in amplitude of muscle contraction, shortening of muscle reaction time between stimulation and contraction. Athlete refers to an individual who participates in sports at any level and who seeks to achieve an improved level of strength, speed and endurance in their performance, such as, for example, body builders, bicyclists, long distance runners, short distance runners, etc. Enhanced sports performance in manifested by the ability to overcome muscle fatigue, ability to maintain activity for longer periods of time, and have a more effective workout.

In the arena of athlete muscle performance, it is desirable to create conditions that permit competition or training at higher levels of resistance for a prolonged period of time.

It is contemplated that the methods of the present invention will also be effective in the treatment of muscle related pathological conditions, including acute sarcopenia, for example, muscle atrophy and/or cachexia associated with burns, bed rest, limb immobilization, or major thoracic, abdominal, and/or orthopedic surgery.

In certain embodiments, the invention provides novel dietary compositions comprising sirtuin modulators, a method for their preparation, and a method of using the compositions for improvement of sports performance. Accordingly, provided are therapeutic compositions, foods and beverages that have actions of improving physical endurance and/or inhibiting physical fatigues for those people involved in broadly-defined exercises including sports requiring endurance and labors requiring repeated muscle exertions. Such dietary compositions may additional comprise electrolytes, caffeine, vitamins, carbohydrates, etc.

### Other Uses

Sirtuin-modulating compounds that increase the level and/or activity of a sirtuin protein may be used, in patients having a T373N or L107X Sirt1 polymorphic variant, for treating or preventing viral infections (such as infections by influenza, herpes or papilloma virus) or as antifungal agents. In certain embodiments, sirtuin-modulating compounds that increase the level and/or activity of a sirtuin protein may be administered as part of a combination drug therapy with another therapeutic agent for the treatment of viral diseases. In another embodiment, sirtuin-modulating compounds that increase the level and/or activity of a sirtuin protein may be administered as part of a combination drug therapy with another anti-fungal agent.

Subjects that may be treated as described herein include eukaryotes, such as mammals, e.g., humans, ovines, bovines, equines, porcines, canines, felines, non-human primate, mice, and rats, having a mutation in a residue equivalent to nucleotide 373 or amino acid 107 of human Sirt1. Cells that may be treated include eukaryotic cells, e.g., from a subject described above.

### 6. Sirtuin Modulating Compounds

In various embodiments, the methods described herein involve administration of a sirtuin modulating compound to a subject having a T373N or L107X Sirt1 polymorphic variant. A sirtuin-modulating compound refers to a compound that may either up regulate (e.g., activate or stimulate), down regulate (e.g., inhibit or suppress) or otherwise change a functional property or biological activity of a sirtuin protein. Sirtuin-modulating compounds may act to modulate a sirtuin protein either directly or indirectly. In certain embodiments, a sirtuin-modulating compound may be a sirtuin-activating compound or a sirtuin-inhibiting compound.

A sirtuin-activating compound refers to a compound that increases the level of a sirtuin protein and/or increases at least one activity of a sirtuin protein. In an exemplary embodiment, a sirtuin-activating compound may increase at least one biological activity of a sirtuin protein by at least about 10%, 25%, 50%, 75%, 100%, or more. In exemplary embodiments, sirtuin activating compounds increase deacetylase activity of a sirtuin protein, e.g., increased deacetylation of one or more sirtuin substrates. Exemplary sirtuin activating compounds include flavones, stilbenes, flavanones, isoflavanones, catechins, chalcones, tannins and anthocyanidins. Exemplary stilbenes include hydroxystilbenes, such as trihydroxystilbenes, e.g., 3,5,4′-trihydroxystilbene (“resveratrol”). Resveratrol is also known as 3,4′,5-stilbenetriol. Tetrahydroxystilbenes, e.g., piceatannol, are also encompassed. Hydroxychalones including trihydroxychalones, such as isoliquiritigenin, and tetrahydroxychalones, such as butein, can also be used. Hydroxyflavones including tetrahydroxyflavones, such as fisetin, and pentahydroxyflavones, such as quercetin, can also be used. Other sirtuin activating compounds are described in U.S. Patent Application Publication No. 2005/0096256 and PCT Application Nos. PCT/US06/002092, PCT/US06/007746, PCT/US06/007744, PCT/US06/007745, PCT/US06/007778, PCT/US06/007656, PCT/US06/007655, PCT/US06/007773, PCT/US06/030661, PCT/US06/030512, PCT/US06/030511, PCT/US06/030510, and PCT/US06/030660.

Methods of determining the activity of a putative sirtuin modulator are known in the art. For example, see PCT Publication No. Wo 2006/094239 and WO 2007/064902. Known methods may be used to assay the activity of different Sirt1 polymorphic variants, including wild-type Sirt1 and Sirt1 having an L107X mutation, wherein X is an amino acid other than leucine.

A sirtuin-inhibiting compound refers to a compound that decreases the level of a sirtuin protein and/or decreases at least one activity of a sirtuin protein. In an exemplary embodiment, a sirtuin-inhibiting compound may decrease at least one biological activity of a sirtuin protein by at least about 10%, 25%, 50%, 75%, 100%, or more. In exemplary embodiments, sirtuin inhibiting compounds decrease deacetylase activity of a sirtuin protein, e.g., decreased deacetylation of one or more sirtuin substrates. Exemplary sirtuin inhibitors include, for example, sirtinol and analogs thereof (see e.g., Napper et al., J. Med. Chem. 48: 8045-54 (2005)), nicotinamide (NAD⁺) and suramin and analogs thereof. Other sirtuin inhibiting compounds are described in U.S. Patent Application Publication No. 2005/0096256, PCT Publication No. WO2005/002527, and PCT Application Nos. PCT/US06/007746, PCT/US06/007744, PCT/US06/007745, PCT/US06/007778, PCT/US06/007656, PCT/US06/007655, PCT/US06/007773 and PCT/US06/007742.

Exemplary sirtuin activating compounds are provided below. In one embodiment, the methods disclosed herein utilize administration of a sirtuin-modulating compound of Formula (I):

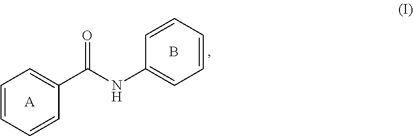

or a salt thereof, where:

Ring A is optionally substituted, fused to another ring or both; and

Ring B is substituted with at least one carboxy, substituted or unsubstituted arylcarboxamine, substituted or unsubstituted aralkylcarboxamine, substituted or unsubstituted heteroaryl group, substituted or unsubstituted heterocyclylcarbonylethenyl, or polycyclic aryl group or is fused to an aryl ring and is optionally substituted by one or more additional groups.

In one embodiment, the methods disclosed herein utilize administration of a sirtuin-modulating compound of Formula (II):

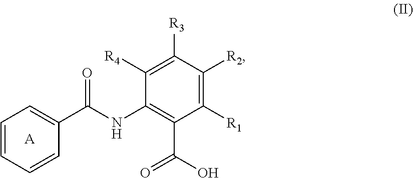

or a salt thereof, where:

Ring A is optionally substituted;

R₁, R₂, R₃ and R₄ are independently selected from the group consisting of —H, halogen, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙR₅, —S(O)ₙOR₅, —S(O)ₙNR₅R₆, —NR₅R₆, —NR₅C(O)OR₆, —NR₅C(O)R₆ and —NO₂;

R₅ and R₆ are independently —H, a substituted or unsubstituted alkyl group, a substituted or unsubstituted aryl group or a substituted or unsubstituted heterocyclic group; and

n is 1 or 2.

I In one embodiment, the methods disclosed herein utilize administration of a sirtuin-modulating compound of Formula (III):

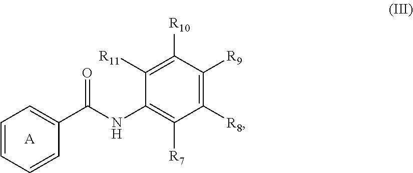

or a salt thereof, where:

Ring A is optionally substituted;

R₅ and R₆ are independently —H, a substituted or unsubstituted alkyl group, a substituted or unsubstituted aryl group or a substituted or unsubstituted heterocyclic group;

R₇, R₉, R₁₀ and R₁₁ are independently selected from the group consisting of —H, halogen, —R₅, —OR₅, —CN, —CO₂R₅, —OCOR₅, —OCO₂R₅, —C(O)NR₅R₆, —OC(O)NR₅R₆, —C(O)R₅, —COR_{S}, —SR₅, —OSO₃H, —S(O)ₙR₅, —S(O)ₙOR₅, —S(O)ₙNR₅R₆, —NR₅R₆, —NR₅C(O)OR₆, —NR₅C(O)R₆ and —NO₂;

R₈ is a polycyclic aryl group; and

n is 1 or 2.

In one embodiment, the methods disclosed herein utilize administration of a sirtuin-modulating compound of Formula (IV):

Ar-L-J-M-K-Ar′ (IV)

or a salt thereof, wherein:

each Ar and Ar′ is independently an optionally substituted carbocyclic or heterocyclic aryl group;

L is an optionally substituted carbocyclic or heterocyclic arylene group;

each J and K is independently NR₁′, O, S, or is optionally independently absent; or when J is NR₁′, R₁′ is a C1-C4 alkylene or C2-C4 alkenylene attached to Ar′ to form a ring fused to Ar′; or when K is NR₁′, R₁′ is a C1-C4 alkylene or C2-C4 alkenylene attached to L to form a ring fused to L;

each M is C(O), S(O), S(O)₂, or CR₁′R₁′;

each R₁′ is independently selected from H, C1-C10 alkyl; C2-C10 alkenyl; C2-C10 alkynyl; C3-C10 cycloalkyl; C4-C10 cycloalkenyl; aryl; R₅′; halo; haloalkyl; CF₃; SR₂′; OR₂′; NR₂′R₂′; NR₂′R₃′; COOR₂′; NO₂; CN; C(O)R₂′; C(O)C(O)R₂′; C(O)NR₂′R₂′; OC(O)R₂′; S(O)₂R₂′; S(O)₂NR₂′R₂′; NR₂′C(O)NR₂′R₂′; NR₂′C(O)C(O)R₂′; NR₂′C(O)R₂′; NR₂′(COOR₂′); NR₂′C(O)R₅′; NR₂′S(O)₂NR₂′R₂′; NR₂′S(O)₂R₂′; NR₂′S(O)₂R₅′; NR₂′C(O)C(O)NR₂′R₂′; NR₂′C(O)C(O)NR₂′R₃′; C1-C10 alkyl substituted with aryl, R₄′ or R₅′; or C2-C10 alkenyl substituted with aryl, R₄′ or R₅′;

each R₂′ is independently H; C1-C10 alkyl; C2-C10 alkenyl; C2-C10 alkynyl; C3-C10 cycloalkyl; C4-C10 cycloalkenyl; aryl; R₆′; C1-C10 alkyl substituted with 1-3 independent aryl, R₄′ or R₆′ groups; C3-C10 cycloalkyl substituted with 1-3 independent aryl, R₄′ or R₆′ groups; or C2-C10 alkenyl substituted with 1-3 independent aryl, R₄′ or R₆′;

each R₃′ is independently C(O)R₂′, COOR₂′, or S(O)₂R₂′;

each R₄′ is independently halo, CF₃, SR₇′, OR₇′, OC(O)R₇′, NR₇′R₇′, NR₇′R₈′, NR₈′R₈′, COOR₇′, NO₂, CN, C(O)R₇′, or C(O)NR₇′R₇′;

each R₅′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; aryl; R₆′; halo; sulfur; oxygen; CF₃; haloalkyl; SR₂′; OR₂′; OC(O)R₂′; NR₂′R₂′; NR₂′R₃′; NR₃′R₃′; COOR₂′; NO₂; CN; C(O)R₂′; C(O)NR₂′R₂′; C1-C10 alkyl substituted with 1-3 independent R₄′, R₆′, or aryl; or C2-C10 alkenyl substituted with 1-3 independent R₄′, R₆′, or aryl;

each R₆ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; halo; sulfur; oxygen; CF₃; haloalkyl; SR₇′; OR₇′; NR₇′R₇′; NR₇′R₈′; NR₈′R₈′; COOR₇′; NO₂; CN; C(O)R₇′; or C(O)NR₇′R₇′;

each R₇′ is independently H, C1-C10 alkyl; C2-C10 alkenyl; C2-C10 alkynyl; C3-C10 cycloalkyl; C4-C10 cycloalkenyl; haloalkyl; C1-C10 alkyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′; or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₈′ is independently C(O)R₇′, COOR₇′, or S(O)₂R₇′;

each R₉′ is independently H, C1-C10 alkyl, C2-C10 alkenyl, C2-C10 alkynyl, C3-C10 cycloalkyl, C4-C10 cycloalkenyl, or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₁₀′ is independently H; C1-C10 alkyl; C2-C10 alkenyl; C2-C10 alkynyl; C3-C10 cycloalkyl; C4-C10 cycloalkenyl; C1-C10 alkyl optionally substituted with halo, CF₃, OR₁₁′, SR₁₁′, NR₁₁′R₁₁′, COOR₁₁′, NO₂, CN; or phenyl optionally substituted with halo, CF₃, OR₁₁′, SR₁₁′, NR₁₁′R₁₁′, COOR₁₁′, NO₂, CN;

each R₁₁′ is independently H; C1-C10 alkyl; C3-C10 cycloalkyl or phenyl;

each haloalkyl is independently a C1-C10 alkyl substituted with one or more halogen atoms, selected from F, Cl, Br, or I, wherein the number of halogen atoms may not exceed that number that results in a perhaloalkyl group; and

each aryl is independently optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′)C(O)R₉′; N(R₉′)(COOR₉′); N(R₉′)S(O)₂R₉′; S(O)₂NR₉′R₉′; OC(O)R₉′; NR₉′C(O)NR₉′R₉′; NR₉′C(O)C(O)R₉′; NR₉′C(O)R₆′; NR₉′S(O)₂NR₉′R₉′; NR₉′S(O)₂R₆′; NR₉′C(O)C(O)NR₉′R₉′; C1-C10 alkyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; C2-C10 alkenyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; or R₉′.

In one embodiment, the methods disclosed herein utilize administration of a sirtuin-modulating compound of Formula (IVa):

Het-L-Q-Ar′ (IVa)

or a salt thereof, where:

Het is an optionally substituted heterocyclic aryl group;

L is an optionally substituted carbocyclic or heterocyclic arylene group;

Ar′ is an optionally substituted carbocyclic or heterocyclic aryl group; and

Q is selected from —NR₁′—C(O)—, —NR₁′—S(O)₂—, —NR₁′—C(O)—NR₁′—, —NR₁′—C(S)—NR₁′—, —NR₁′—C(O)—CR₁′R′₁—NR₁′—, —NR₁′—C(═NR₁′)—NR₁′—, —C(O)—NR₁′—, —C(O)—NR₁′—S(O)₂—, —NR₁′—, —CR₁′R′₁—, —NR₁′—C(O)—CR₁′═CR₁′—, —NR₁′—S(O)₂—NR₁′—, —NR₁′—C(O)—NR₁′—S(O)₂—, —NR₁′—CR₁′R′₁—C(O)—NR₁′—, —CR₁′R′₁—C(O)—NR₁′—, —NR₁′—C(O)—CR₁′═CR₁′—CR₁′R₁′—, —NR₁′—C(═N—CN)—NR₁′—, —NR₁′—C(O)—CR₁′R′₁—,

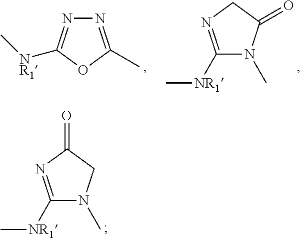

and

each R₁′ is independently selected from H or optionally substituted C₁-C₃ straight or branched alkyl, wherein:

when Het is a polycyclic heteroaryl, L is an optionally substituted phenylene, Q and Het are attached to L in a meta orientation, and Ar′ is optionally substituted phenyl; then Q is not —NH—C(O)—.

In one embodiment, the methods disclosed herein utilize administration of a sirtuin-modulating compound of Formula (V):

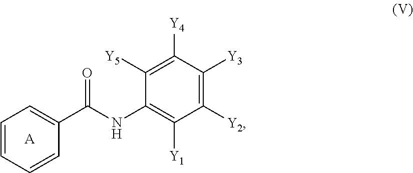

or a salt thereof, wherein:

Ring A is optionally substituted with at least one R₁′ group;

Y₁, Y₂, Y₃, Y₄, and Y₅ are independently R₁′;

each R₁′ is independently selected from H, C1-C10 alkyl; C2-C10 alkenyl; C2-C10 alkynyl; C3-C10 cycloalkyl; C4-C10 cycloalkenyl; aryl; R₅′; halo; haloalkyl; CF₃; SR₂′; OR₂′; NR₂′R₂′; NR₂′R₃′; COOR₂′; NO₂; CN; C(O)R₂′; C(O)C(O)R₂′; C(O)NR₂′R₂′; OC(O)R₂′; S(O)₂R₂′; S(O)₂NR₂′R₂′; NR₂′C(O)NR₂′R₂′; NR₂′C(O)C(O)R₂′; NR₂′C(O)R₂′; NR₂′(COOR₂′); NR₂′C(O)R₅′; NR₂′S(O)₂NR₂′R₂′; NR₂′S(O)₂R₂′; NR₂′S(O)₂R₅′; NR₂′C(O)C(O)NR₂′R₂′; NR₂′C(O)C(O)NR₂′R₃′; C1-C10 alkyl substituted with aryl, R₄′ or R₅′; or C2-C10 alkenyl substituted with aryl, R₄′ or R₅′;

each R₂′ is independently H; C1-C10 alkyl; C2-C10 alkenyl; C2-C10 alkynyl; C3-C10 cycloalkyl; C4-C10 cycloalkenyl; aryl; R₆′; C1-C10 alkyl substituted with 1-3 independent aryl, R₄′ or R₆′ groups; C3-C10 cycloalkyl substituted with 1-3 independent aryl, R₄′ or R₆′ groups; or C2-C10 alkenyl substituted with 1-3 independent aryl, R₄′ or R₆′;

each R₃′ is independently C(O)R₂′, COOR₂′, or S(O)₂R₂′;

each R₄′ is independently halo, CF₃, SR₇′, OR₇′, OC(O)R₇′, NR₇′R₇′, NR₇′R₈′, NR₈′R₈′, COOR₇′, NO₂, CN, C(O)R₇′, or C(O)NR₇′R₇′;

each R₅′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; aryl; R₆′; halo; sulfur; oxygen; CF₃; haloalkyl; SR₂′; OR₂′; OC(O)R₂′; NR₂′R₂′; NR₂′R₃′; NR₃′R₃′; COOR₂′; NO₂; CN; C(O)R₂′; C(O)NR₂′R₂′; C1-C10 alkyl substituted with 1-3 independent R₄′, R₆′, or aryl; or C2-C10 alkenyl substituted with 1-3 independent R₄′, R₆′, or aryl;

each R₆′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; halo; sulfur; oxygen; CF₃; haloalkyl; SR₇′; OR₇′; NR₇′R₇′; NR₇′R₈′; NR₈′R₈′; COOR₇′; NO₂; CN; C(O)R₇′; or C(O)NR₇′R₇′;

each R₇′ is independently H, C1-C10 alkyl; C2-C10 alkenyl; C2-C10 alkynyl; C3-C10 cycloalkyl; C4-C10 cycloalkenyl; haloalkyl; C1-C10 alkyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′; or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₈′ is independently C(O)R₇′, COOR₇′, or S(O)₂R₇′;

each R₉′ is independently H, C1-C10 alkyl, C2-C10 alkenyl, C2-C10 alkynyl, C3-C10 cycloalkyl, C4-C10 cycloalkenyl, or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₁₀′ is independently H; C1-C10 alkyl; C2-C10 alkenyl; C2-C10 alkynyl; C3-C10 cycloalkyl; C4-C10 cycloalkenyl; C1-C10 alkyl optionally substituted with halo, CF₃, OR₁₁′, SR₁₁′, NR₁₁′R₁₁′, COOR₁₁′, NO₂, CN; or phenyl optionally substituted with halo, CF₃, OR₁₁′, SR₁₁′, NR₁₁′R₁₁′, COOR₁₁′, NO₂, CN;

each R₁₁′ is independently H; C1-C10 alkyl; C3-C10 cycloalkyl or phenyl;

each haloalkyl is independently a C1-C10 alkyl substituted with one or more halogen atoms, selected from F, Cl, Br, or I, wherein the number of halogen atoms may not exceed that number that results in a perhaloalkyl group; and

each aryl is independently a 5- to 7-membered monocyclic ring system or a 9- to 12-membered bicyclic ring system optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′)C(O)R₉′; N(R₉′)(COOR₉′); N(R₉′)S(O)₂R₉′; S(O)₂NR₉′R₉′; OC(O)R₉′; NR₉′C(O)NR₉′R₉′; NR₉′C(O)C(O)R₉′; NR₉′C(O)R₆′; NR₉′S(O)₂NR₉′R₉′; NR₉′S(O)₂R₆′; NR₉′C(O)C(O)NR₉′R₉′; C1-C10 alkyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; C2-C10 alkenyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; or R₉′.

In one embodiment, the methods disclosed herein utilize administration of a sirtuin-modulating compound of Formula (VI):

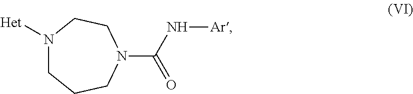

or a salt thereof, wherein:

Het is an optionally substituted heterocyclic aryl group; and

Ar′ is an optionally substituted carbocyclic or heterocyclic aryl group.

The invention also includes prodrugs and metabolites of the compounds disclosed herein.

In one embodiment, the methods disclosed herein utilize administration of a sirtuin-modulating compound of Formula (VII):

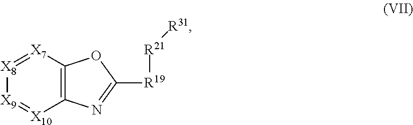

or a salt thereof, wherein:

each of X₇, X₈, X₉ and X₁₀ is independently selected from N, CR²⁰, or CR₁′, wherein:

each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

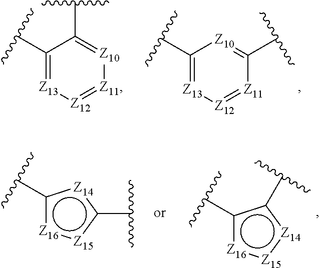

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R′₁—CR₁′R′₁—, —NR₁′—C(S)—NR₁′—CR₁′R′₁—CR₁′R′₁—, —NR₁′—C(O)—O—,

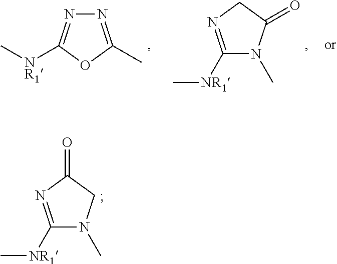

and

R³¹ is selected from an optionally substituted monocyclic or bicyclic aryl, or an optionally substituted monocyclic or bicyclic heteroaryl, with the provisos that said compound is not:

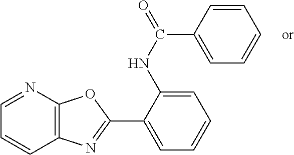

that when R¹⁹ is

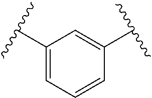

and R₂₁ is —NHC(O)—, R³¹ is not an optionally substituted phenyl.

In certain embodiments, compounds of Structural Formula (VII) have the following values:

each of X₇, X₈, X₉ and X₁₀ is independently selected from N, CR²⁰, or CR₁′, wherein:

each R²⁰ is independently selected from H or a solubilizing group;

each R₁′ is independently selected from H or optionally substituted C₁-C₃ straight or branched alkyl;

one of X₇, X₈, X₉ and X₁₀ is N and the others are selected from CR²⁰ or CR₁′; and

zero to one R²⁰ is a solubilizing group;

R¹⁹ is selected from:

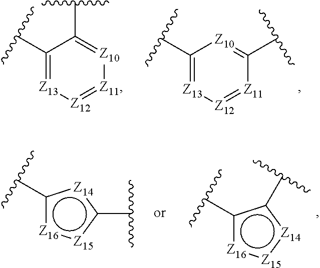

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′, —C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

said compound is not:

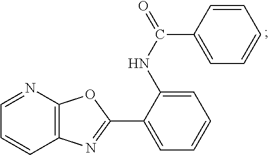

and

when X₈ and X₉ are each independently selected from CR²⁰ or CR₁′, R¹⁹ is

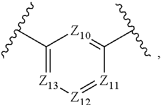

and each of Z₁₀, Z₁₁, Z₁₂ and Z₁₃ is independently selected from CR²⁰, or CR₁′, then:
a) at least one of X₈ and X₉ is not CH; or
b) at least one of Z₁₀, Z₁₁, Z₁₂ and Z₁₃ is CR²⁰, wherein R²⁰ is a solubilizing group.

In one embodiment, the methods disclosed herein utilize administration of a sirtuin-modulating compound of Formula (VIII):

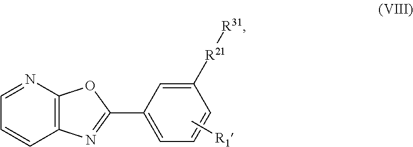

or a salt thereof, wherein:

R₁′ is selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R′₁—CR₁′R′₁—, —NR₁′—C(S)—NR₁′—CR₁′R′₁—CR₁′R′₁—, —NR₁′—C(O)—O—,

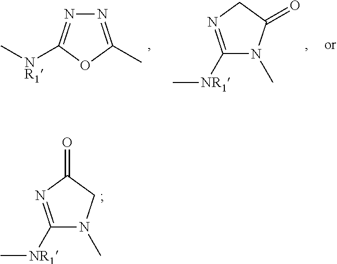

and

R³¹ is selected from an optionally substituted monocyclic or bicyclic aryl, or an optionally substituted monocyclic or bicyclic heteroaryl, with the provisos that:

when R₁′ is methyl, and R²¹ is —NH—C(O)—, R³¹ is not

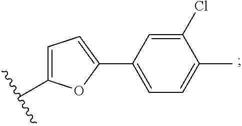

1-methoxynaphthyl; 2-methoxynaphthyl; or unsubstituted 2-thienyl;

when R₁′ is methyl, and R²¹ is —NH—C(O)—CH═CH—, R³¹ is not

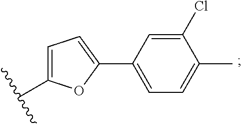

when R₁′ is methyl, and R²¹ is —NH—C(O)—CH—O—, R³¹ is not unsubstituted naphthyl; 2-methoxy, 4-nitrophenyl; 4-chloro, 2-methylphenyl; or 4-t-butylphenyl; and

when R²¹ is —NH—C(O)—, R³¹ is not optionally substituted phenyl.

In one embodiment, the methods disclosed herein utilize administration of a sirtuin-modulating compound of Formula (IX):

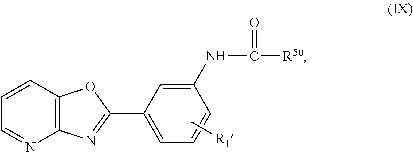

or a salt thereof, wherein:

R₁′ is selected from H or optionally substituted C₁-C₃ straight or branched alkyl; and

R⁵⁰ is selected from 2,3-dimethoxyphenyl, phenoxyphenyl, 2-methyl-3-methoxyphenyl, 2-methoxy-4-methylphenyl, or phenyl substituted with 1 to 3 substituents, wherein one of said substituents is a solubilizing group; with the provisos that R⁵⁰ is not substituted simultaneously with a solubilizing group and a nitro group, and R⁵⁰ is not singly substituted at the 4-position with cyclic solubilizing group or at the 2-position with a morpholino group.

In one embodiment, the methods disclosed herein utilize administration of a sirtuin-modulating compound of Formula (X):

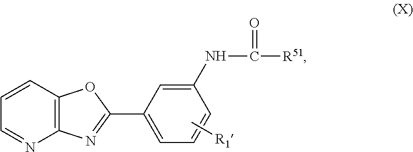

or a salt thereof, wherein:

R₁′ is selected from H or optionally substituted C₁-C₃ straight or branched alkyl; and

R⁵¹ is selected from an optionally substituted monocyclic heteroaryl, an optionally substituted bicyclic heteroaryl, or an optionally substituted naphthyl, wherein R⁵¹ is not chloro-benzo(b)thienyl, unsubstituted benzodioxolyl, unsubstituted benzofuranyl, methyl-benzofuranyl, unsubstituted furanyl, phenyl-, bromo-, or nitro-furyl, chlorophenyl-isoxazolyl, oxobenzopyranyl, unsubstituted naphthyl, methoxy-, methyl-, or halo-naphthyl, unsubstituted thienyl, unsubstituted pyridinyl, or chloropyridinyl.

In one embodiment, the methods disclosed herein utilize administration of a sirtuin-modulating compound of Formula (XI):

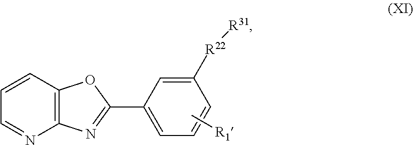

or a salt thereof, wherein:

R₁′ is selected from H or optionally substituted C₁-C₃ straight or branched alkyl;

R²² is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R′₁—CR₁′R′₁—, —NR₁′—C(S)—NR₁′—CR₁′R′₁—CR₁′R′₁—, —NR₁′—C(O)—O— or —NR₁′—C(O)—CR₁′R₁′—, wherein R²³ is an optionally substituted C₁-C₃ straight or branched alkyl; and

R³¹ is selected from an optionally substituted monocyclic or bicyclic aryl, or an optionally substituted monocyclic or bicyclic heteroaryl, with the provisos that:

when R²² is —NH—C(O)—CH═CH—, R³¹ is not unsubstituted furyl, 5-(2-methyl-3-chlorophenyl)-furanyl, 2,4-dichlorophenyl, 3,5-dichloro-2-methoxyphenyl, 3-nitrophenyl, 4-chlorophenyl, 4-chloro-3-nitrophenyl, 4-isopropylphenyl, 4-methoxyphenyl, 2-methoxy-5-bromophenyl, or unsubstituted phenyl;

when R²² is —NH—C(O)—CH₂—, R³¹ is not 3,4-dimethoxyphenyl, 4-chlorophenyl, or unsubstituted phenyl;

when R²² is —NH—C(O)—CH₂—O—, R³¹ is not 2,4-dimethyl-6-nitrophenyl, 2- or 4-nitrophenyl, 4-cyclohexylphenyl, 4-methoxyphenyl, unsubstituted naphthyl, or unsubstituted phenyl, or phenyl monosubstituted, disubstituted or trisubstituted solely with substituents selected from straight- or branched-chain alkyl or halo;

when R²² is —NH—C(O)—CH(CH₃)—O—, R³¹ is not 2,4-dichlorophenyl, 4-chlorophenyl, or unsubstituted phenyl; and

when R²² is —NH—S(O)₂—, R³¹ is not unsubstituted phenyl.

In one embodiment, the methods disclosed herein utilize administration of a sirtuin-modulating compound of Formula (XII):

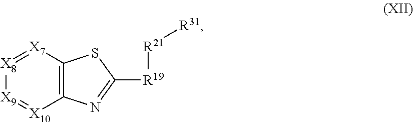

or a salt thereof, wherein:
each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

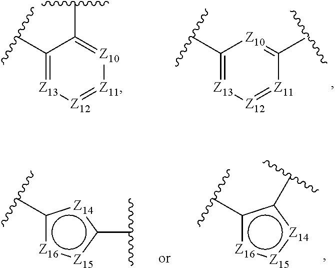

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R′₁, —, —NR₁′—C(S)—NR₁′—CR₁′R′₁, —CR₁′R′₁—, —NR₁′—C(O)—O—,

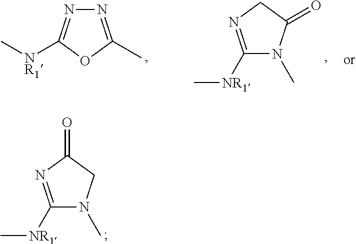

and

R³¹ is selected from an optionally substituted monocyclic or bicyclic aryl, or an optionally substituted monocyclic or bicyclic heteroaryl,

with the proviso that when R¹⁹ is

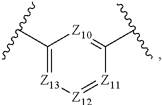

Z₁₀, Z₁₁, Z₁₂ and Z₁₃ are each CH, and R²¹ is —NHC(O)—, R³¹ is not an optionally substituted phenyl.

In certain embodiments, the compounds of Structural Formula (XI) have the following values:

each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

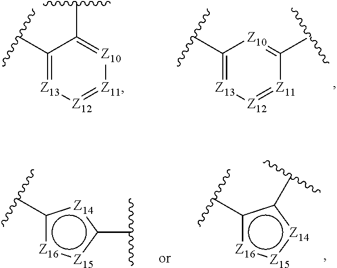

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R′₁—CR₁′R′₁—, —NR₁′—C(S)—NR₁′—CR₁′R′₁—CR₁′R′₁—, —NR₁′—C(O)—O— or —NR₁′—C(O)—CR₁′R₁′—; and

R³¹ is selected from an optionally substituted monocyclic or bicyclic aryl, or an optionally substituted monocyclic or bicyclic heteroaryl, with the proviso that:

when X₇ is N, R¹⁹ is

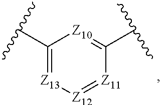

and each of Z₁₀, Z₁₁, Z₁₂ and Z₁₃ is independently selected from CR²⁰, or CR₁′, then:
a) at least one of X₈, X₉ or X₁₀ is C—(C₁-C₃ straight or branched alkyl) or C-(solubilizing group); or
b) at least one of Z₁₀, Z₁₁, Z₁₂ and Z₁₃ is CR²⁰, wherein R²⁰ is a solubilizing group.

In one embodiment, the methods disclosed herein utilize administration of a sirtuin-modulating compound of Formula (XIII):

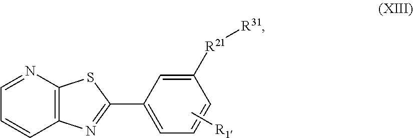

or a salt thereof, wherein:

R₁′ is selected from H or optionally substituted C1-C3 straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R′₁—CR₁′R′₁—, —NR₁′—C(S)—NR₁′—CR₁′R′₁—CR₁′R′₁—, —NR₁′—C(O)—O—,

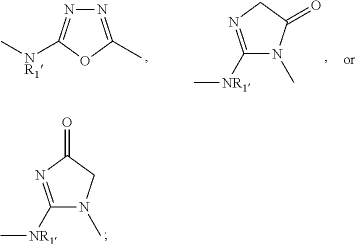

and

R³¹ is selected from an optionally substituted monocyclic or bicyclic aryl, or an optionally substituted monocyclic or bicyclic heteroaryl, with the provisos that:

when R²¹ is —NH—C(O)—, R³¹ is not unsubstituted furyl, 5-bromofuryl, unsubstituted phenyl, phenyl monosubstituted with halo or methyl, 3- or 4-methoxyphenyl, 4-butoxyphenyl, 4-t-butylphenyl, 3-trifluoromethylphenyl, 2-benzoylphenyl, 2- or 4-ethoxyphenyl, 2,3-, 2,4-, 3,4-, or 3,5-dimethoxyphenyl, 3,4,5-trimethoxyphenyl, 2,4- or 2-6 difluorophenyl, 3,4-dioxymethylene phenyl, 3,4- or 3,5-dimethlyphenyl, 2-chloro-5-bromophenyl, 2-methoxy-5-chlorophenyl, unsubstituted quinolinyl, thiazolyl substituted simultaneously with methyl and phenyl, or ethoxy-substituted pyridinyl;

when R²¹ is —NH—C(O)—CH(CH₂—CH₃)—, R³¹ is not unsubstituted phenyl;

when R²¹ is —NH—C(O)—CH₂—, R³¹ is not unsubstituted phenyl, 3-methylphenyl, 4-chlorophenyl, 4-ethoxyphenyl, 4-fluorophenyl or 4-methoxyphenyl;

when R²¹ is —NH—C(O)—CH₂—O—, R³¹ is not unsubstituted phenyl or 4-chlorophenyl; and

when R²¹ is —NH—S(O)₂—, R³¹ is not 3,4-dioxymethylene phenyl, 2,4,5-trimethylphenyl, 2,4,6-trimethylphenyl, 2,4- or 3,4-dimethylphenyl, 2,5-difluorophenyl, 2,5- or 3,4-dimethoxyphenyl, fluorophenyl, 4-chlorophenyl, 4-bromophenyl, 4-ethylphenyl, 4-methylphenyl, 3-methyl-4-methoxyphenyl, unsubstituted phenyl, unsubstituted pyridinyl, unsubstituted thienyl, chloro-substituted thienyl, or methyl-substituted benzothiazolyl.

In one embodiment, the methods disclosed herein utilize administration of a sirtuin-modulating compound of Formula (XIV):

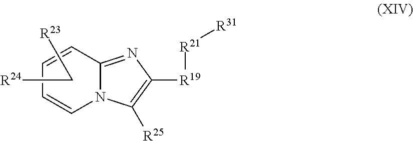

or a salt thereof, wherein:

each of R²³ and R²⁴ is independently selected from H, —CH₃ or a solubilizing group;

R²⁵ is selected from H, or a solubilizing group; and

R¹⁹ is selected from:

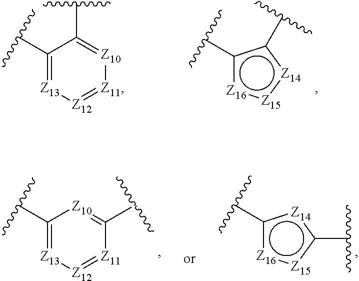

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group; and
zero to one R₁′ is an optionally substituted C₁-C₃ straight or branched alkyl;
each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R′₁—CR₁′R′₁—, —NR₁′—C(S)—NR₁′—CR₁′R′₁—CR₁′R′₁—, —NR₁′—C(O)—O—,

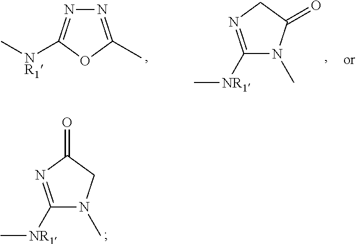

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl,
wherein when R¹⁹ is

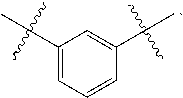

R²¹ is —NH—C(O)— and R²⁵ is —H, R³¹ is not an optionally substituted phenyl group, and wherein said compound is not 2-chloro-N-[3-[3-(cyclohexylamino)imidazo[1,2-a]pyridin-2-yl]phenyl]-4-nitrobenzamide.

In another aspect, the invention provides sirtuin-modulating compounds of Structural Formula (XV):

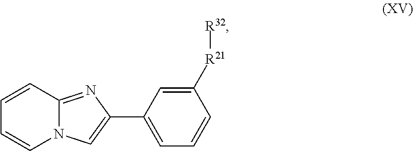

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; NR₁′—C(O)—CR₁′R′₁—CR₁′R′₁—, —NR₁′—C(S)—NR₁′—CR₁′R′₁—CR₁′R′₁—, —NR₁′—C(O)—O—,

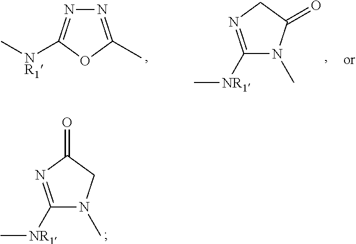

and

each R₁′ is independently selected from H or optionally substituted C₁-C₃ straight or branched alkyl; and

R³² is selected from an optionally substituted bicyclic aryl, or an optionally substituted monocyclic or bicyclic heteroaryl, wherein:

when R²¹ is —NH—C(O)—, R³² is not unsubstituted 2-furyl, 2-(3-bromofuryl), unsubstituted 2-thienyl, unsubstituted 3-pyridyl, unsubstituted 4-pyridyl,

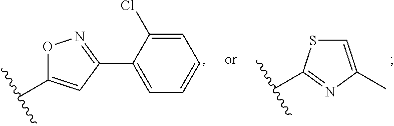

and

when R²¹ is —NR₁′—S(O)₂—, R³² is not unsubstituted 2-thienyl or unsubstituted naphthyl.

In one embodiment, the methods disclosed herein utilize administration of a sirtuin-modulating compound of Formula (XVI):

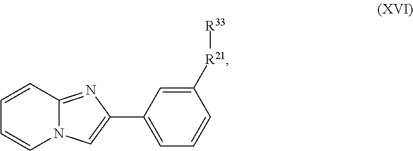

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R′₁—CR₁′R′₁—, —NR₁′—C(S)—NR₁′—CR₁′R′₁—CR₁′R′₁—, —NR₁′—C(O)—O—,

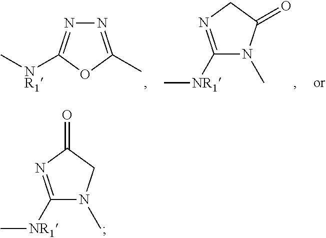

and

each R₁′ is independently selected from H or optionally substituted C₁-C₃ straight or branched alkyl; and

R³³ is an optionally substituted phenyl, wherein:

when R²¹ is —NH—C(O)—, R³³ is a substituted phenyl other than phenyl singly substituted with halo, methyl, nitro or methoxy; 2-carboxyphenyl; 4-n-pentylphenyl; 4-ethoxyphenyl; 2-carboxy-3-nitrophenyl; 2-chloro-4-nitrophenyl; 2-methoxy-5-ethylphenyl; 2,4-dimethoxyphenyl; 3,4,5-trimethoxyphenyl; 2,4 dichlorophenyl; 2,6-difluorophenyl; 3,5-dinitrophenyl; or 3,4-dimethylphenyl;

when R²¹ is —NR₁′—C(O)—CR₁′R₁′— or —NH—C(O)—CH(CH₃)—O, R³³ is a substituted phenyl;

when R²¹ is —NH—C(O)—CH₂, R³³ is not unsubstituted phenyl, 4-methoxyphenyl; 3,4-dimethoxyphenyl or 4-chlorophenyl;

when R²¹ is —NH—C(O)—CH₂—O, R³³ is not 2,4-bis(1,1-dimethylpropyl)phenyl;

when R²¹ is —NH—C(O)—NH—, R³³ is not 4-methoxyphenyl; and

when R²¹ is —NH—S(O)₂—, R³³ is a substituted phenyl other than 3-methylphenyl, 3-trifluoromethylphenyl, 2,4,5- or 2,4,6-trimethylphenyl, 2,4- or 3,4-dimethylphenyl, 2,5- or 3,4-dimethoxyphenyl, 2,5-dimethoxy-4-chlorophenyl, 3,6-dimethoxy, 4-methylphenyl, 2,5- or 3,4-dichlorophenyl, 2,5-diethoxyphenyl, 2-methyl-5-nitrophenyl, 2-ethoxy-5-bromophenyl, 2-methoxy-5-bromophenyl, 2-methoxy-3,4-dichlorophenyl, 2-methoxy-4-methyl-5-bromophenyl, 3,5-dinitro-4-methylphenyl, 3-methyl-4-methoxyphenyl, 3-nitro-4-methylphenyl, 3-methoxy-4-halophenyl, 3-methoxy-5-chlorophenyl, 4-n-butoxyphenyl, 4-halophenyl, 4-ethylphenyl, 4-methylphenyl, 4-nitrophenyl, 4-ethoxyphenyl, 4-acetylaminophenyl, 4-methoxyphenyl, 4-t-butylphenyl, or para-biphenyl.

In a further aspect, the invention provides sirtuin-modulating compounds of Structural Formula (XVII):

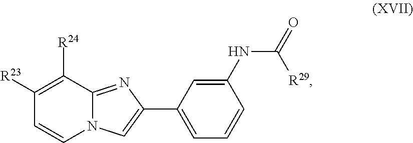

or a salt thereof, wherein:

each of R²³ and R²⁴ is independently selected from H or —CH₃, wherein at least one of R²³ and R²⁴ is H; and

R²⁹ is phenyl substituted with:

a) two —O—CH₃ groups;

b) three —O—CH₃ groups located at the 2,3 and 4 positions; or

c) one —N(CH₃)₂ group; and;

d) when R²³ is CH₃, one —O—CH₃ group at the 2 or 3 position,

wherein R²⁹ is optionally additionally substituted with a solubilizing group.

In one embodiment, the methods disclosed herein utilize administration of a sirtuin-modulating compound of Formula (XVIII):

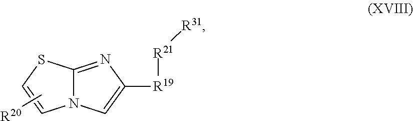

or a salt thereof, wherein

R¹⁹ is selected from:

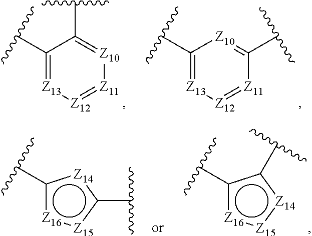

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:

zero to two of Z₁₀, Z₁₁, Z₁₂ or Z₁₃ are N;

at least one of Z₁₄, Z₁₅ and Z₁₆ is N, NR₁′, S or O;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group; and

zero to one R₁′ is an optionally substituted C₁-C₃ straight or branched alkyl;

each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R′₁—CR₁′R′₁—, —NR₁′—C(S)—NR₁′—CR₁′R′₁—CR₁′R′₁—, —NR₁′—C(O)—O—,

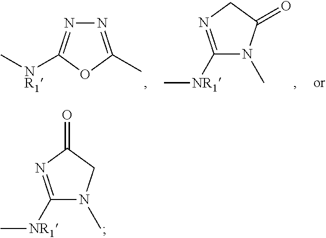

wherein each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl, with the proviso that when R¹⁹ is

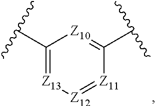

Z₁₀, Z₁₁, Z₁₂ and Z₁₃ are each CH, R²⁰ is H, and R²¹ is —NHC(O)—, R³¹ is not an optionally substituted phenyl.

In another aspect, the invention provides sirtuin-modulating compounds of Structural Formula (XX):

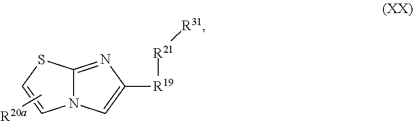

or a salt thereof, wherein

R¹⁹ is selected from:

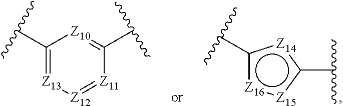

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group; and
zero to one R₁′ is an optionally substituted C₁-C₃ straight or branched alkyl;

each R²⁰ is independently selected from H or a solubilizing group;

R^{20a} is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂₋₅—NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R′₁—CR₁′R′₁—, —NR₁′—C(S)—NR₁′—CR₁′R′₁—CR₁′R′₁—, —NR₁′—C(O)—O—,

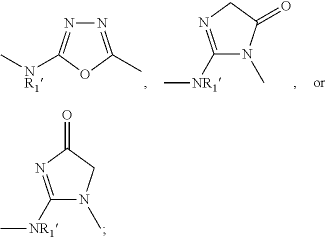

wherein
each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl, wherein when R¹⁹ is

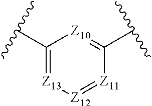

and Z₁₀, Z₁₁, Z₁₂ and Z₁₃ are each CH, R^{20a} is a solubilizing group.

In one embodiment, the methods disclosed herein utilize administration of a sirtuin-modulating compound of Formula (XXI):

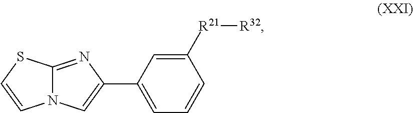

or a salt thereof, wherein

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R′₁—CR₁′R′₁—, —NR₁′—C(S)—NR₁′—CR₁′R′₁—CR₁′R′₁—, —NR₁′—C(O)—O—,

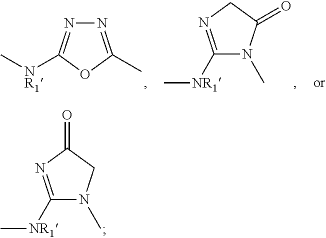

wherein
each R₁′ is independently selected from H or optionally substituted C₁-C₃ straight or branched alkyl; and

R³² is an optionally substituted monocyclic or bicyclic heteroaryl, or an optionally substituted bicyclic aryl, wherein:

when R²¹ is —NH—C(O)—CH₂—, R³² is not unsubstituted thien-2-yl;

when R²¹ is —NH—C(O)—, R³² is not furan-2-yl, 5-bromofuran-2-yl, or 2-phenyl-4-methylthiazol-5-yl;

when R²¹ is —NH—S(O)₂—, R³² is not unsubstituted naphthyl or 5-chlorothien-2-yl.

In one embodiment, the methods disclosed herein utilize administration of a sirtuin-modulating compound of Formula (XXII):

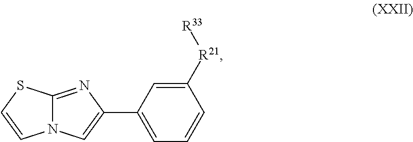

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R′₁—CR₁′R′₁—, —NR₁′—C(S)—NR₁′—CR₁′R′₁—CR₁′R′₁—, —NR₁′—C(O)—O—,

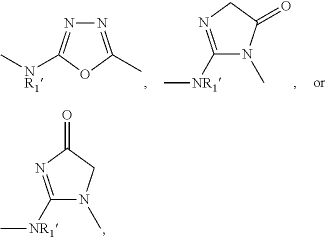

wherein each R₁′ is independently selected from H or optionally substituted C₁-C₃ straight or branched alkyl; and

R³³ is an optionally substituted phenyl, wherein:

when R²¹ is —NR₁′—C(O)—, R₁′ is not H;

when R²¹ is —NH—C(O)—CH₂ or —NH—C(O)—CH₂—O—, R³³ is not unsubstituted phenyl or 4-halophenyl; and

when R²¹ is —NH—S(O)₂—, R³³ is not unsubstituted phenyl, 2,4- or 3,4-dimethylphenyl, 2,4-dimethyl-5-methoxyphenyl, 2-methoxy-3,4-dichlorophenyl, 2-methoxy, 5-bromophenyl-3,4-dioxyethylenephenyl, 3,4-dimethoxyphenyl, 3,4-dichlorophenyl, 3,4-dimethylphenyl, 3- or 4-methylphenyl, 4-alkoxyphenyl, 4-phenoxyphenyl, 4-halophenyl, 4-biphenyl, or 4-acetylaminophenyl.

In one embodiment, the methods disclosed herein utilize administration of a sirtuin-modulating compound of Formula (XXII):

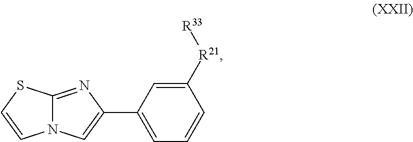

or a salt thereof wherein:

R²¹ is selected from —NH—C(O)—, or —NH—C(O)—CH₂—; and

R³³ is phenyl substituted with

a) one —N(CH₃)₂ group;

b) one CN group at the 3 position;

c) one —S(CH₃) group; or

d)

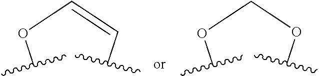

bridging the 3 and 4 positions.

In one embodiment, the methods disclosed herein utilize administration of a sirtuin-modulating compound of Formula (XOH):

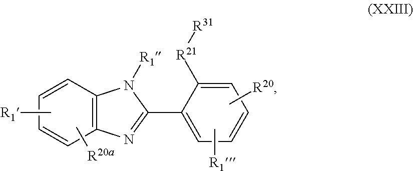

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R′₁—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R′₁—C(O)—NR₁′—, —NR₁′—C(O)—CR₁′═CR₁′—CR₁′R₁′—, —NR₁′—C(═N—CN)—NR₁′—, —NR₁′—C(O)—CR₁′R′₁—O—, —NR₁′—C(O)—CR₁′R₁′—CR₁′R₁′—O—, —NR₁′—S(O)₂—CR₁′R′₁—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—, R³¹ is not is not 3,5-dinitrophenyl, 4-butoxyphenyl,

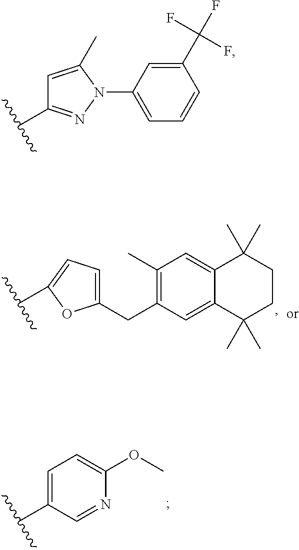

when R²¹ is —NH—C(O)— and each of R²⁰, R^{20a}, R₁′, R₁″ and R₁′″ is hydrogen, R³¹ is not

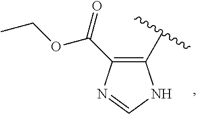

unsubstituted phenyl, 2- or 4-nitrophenyl, 2,4-dinitrophenyl, 2- or 4-chlorophenyl, 2-bromophenyl, 4-fluorophenyl, 2,4-dichlorophenyl, 2-carboxyphenyl, 2-azidophenyl, 2- or 4-aminophenyl, 2-acetamidophenyl, 4-methylphenyl, or 4-methoxyphenyl;

when R²¹ is —NH—C(O)—, R₁″ is methyl; and each of R²⁰, R^{20a}, R₁′ and R₁′″ is hydrogen, R³¹ is not 2-methylaminophenyl,

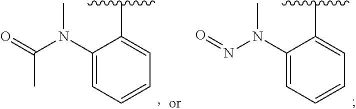

when R²¹ is —NH—C(O)—CH₂— or NH—C(S)—NH—, and each of R²⁰, R^{20a}, R₁′, R₁″ and R₁′″ is hydrogen, R³¹ is not unsubstituted phenyl;

when R²¹ is —NH—S(O)₂—, R₁″ is hydrogen or methyl, and each of R²⁰, R^{20a}, R₁′ and R₁′″ is hydrogen, R³¹ is not 4-methylphenyl; and

when R²¹ is —NH—S(O)₂—, R^{20a} is hydrogen or —CH₂—N(CH₂CH₃)₂, and each of R²⁰, R₁′, R₁″ and R₁′″ is hydrogen, R³¹ is not

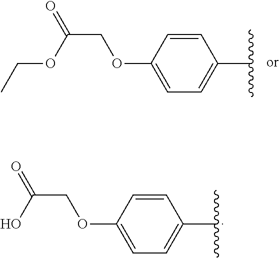

In one embodiment, the methods disclosed herein utilize administration of a sirtuin-modulating compound of Formula (XOH):

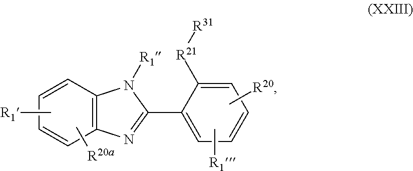

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R′₁—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R′₁—C(O)—NR₁′—, —NR₁′—C(O)—CR₁′═CR₁′—CR₁′R₁′—, —NR₁′—C(═N—CN)—NR₁′—, —NR₁′—C(O)—CR₁′R′₁—O—, —NR₁′—C(O)—CR₁′R₁′—CR₁′R₁′—O—, —NR₁′—S(O)₂—CR₁′R′₁—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, wherein:

i) at least one R²⁰ is a solubilizing group or at least one R₁′″ is an optionally substituted C₁-C₃ straight or branched alkyl or both; or
ii) R^{20a} is a solubilizing group other than CH₂—N(CH₂CH₃)₂.

In one embodiment, the methods disclosed herein utilize administration of a sirtuin-modulating compound of Formula (XXIV):

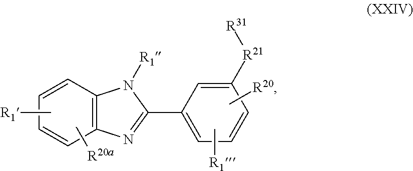

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl;

R²¹ is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R′₁, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R′₁—C(O)—NR₁′—, —NR₁′—C(O)—CR₁′═CR₁′—CR₁′R₁′—, —NR₁′—C(═N—CN)—NR₁′—, —NR₁′—C(O)—CR₁′R′₁—O—, —NR₁′—C(O)—CR₁′R₁′—CR₁′R₁′—O—, —NR₁′—S(O)₂—CR₁′R′₁—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—CH₂—, R³¹ is not 2-methylphenyl, or 3,4-dimethoxyphenyl;

when R²¹ is —NH—C(O)—CH═CH—, R³¹ is not 2-chlorophenyl;

when R²¹ is —NH—C(O)—NH—, R³¹ is not unsubstituted benzimidazolyl;

when R²¹ is —NH—S(O)₂—, and each of R²⁰, R^{20a}, R₁′, R₁″and R₁′″ is hydrogen, R³¹ is not unsubstituted phenyl, 4-chlorophenyl, 4-methylphenyl, or 4-acetoamidophenyl;

when R²¹ is —NH—S(O)₂—, each of R₁′ and R₁′″ is methyl or hydrogen, and each of R²⁰, R^{20a}, and R₁″ is hydrogen, R³¹ is not 4-nitrophenyl;

when R²¹ is —NH—C(O)—CH₂—O—, R₁′″ is methyl or hydrogen, and each of R²⁰, R^{20a}, R₁′, and R₁″ is hydrogen, R³¹ is not 2,3-, 2,5-, 2,6-, 3,4- or 3,5-dimethylphenyl, 2,4-dichloromethyl, 2,4-dimethyl-6-bromophenyl, 2- or 4-chlorophenyl, 2-(1-methylpropyl)phenyl, 5-methyl-2-(1-methylethyl)phenyl, 2- or 4-methylphenyl, 2,4-dichloro-6-methylphenyl, nitrophenyl, 2,4-dimethyl-6-nitrophenyl, 2- or 4-methoxyphenyl, 4-acetyl-2-methoxyphenyl, 4-chloro-3,5-dimethylphenyl, 3-ethylphenyl, 4-bromophenyl, 4-cyclohexyphenyl, 4-(1-methylpropyl)phenyl, 4-(1-methylethyl)phenyl, 4-(1,1-dimethylethyl)phenyl, or unsubstituted phenyl;

when R²¹ is —NH—C(O)—CH₂—, R₁′″ is methyl or hydrogen, and each of R²⁰, R^{20a}, R₁′, and R₁″ is hydrogen, R³¹ is not unsubstituted naphthyl, 4-chlorophenyl, 4-nitrophenyl, 4-methoxyphenyl, unsubstituted phenyl, unsubstituted thienyl

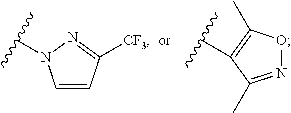

when R²¹ is —NH—C(O)—CH₂—, R₁′ is methyl, and each of R²⁰, R^{20a}, R₁″, and R₁′″ is hydrogen, R³¹ is not unsubstituted phenyl;

when R²¹ is —NH—C(O)—CH═CH, R₁′″ is methyl or hydrogen, and each of R²⁰, R^{20a}, R₁′, and R₁″ is hydrogen, R³¹ is not unsubstituted furyl, nitrophenyl-substituted furyl, 2,4-dichlorophenyl, 3,5-dichloro-2-methoxyphenyl, 3- or 4-nitrophenyl, 4-methoxyphenyl, unsubstituted phenyl, or nitro-substituted thienyl;

when R²¹ is —NH—C(O)—CH(CH₂CH₃)—, and each of R²⁰, R^{20a}, R₁′, R₁″, and R₁′″ is hydrogen, R³¹ is not unsubstituted phenyl;

when R²¹ is —NH—C(O)—CH(CH₃)—O—, R₁′″ is methyl or hydrogen, and each of R²⁰, R^{20a}, R₁′, and R₁″ is hydrogen, R³¹ is not 2,4-dichlorophenyl.

In one embodiment, the methods disclosed herein utilize administration of a sirtuin-modulating compound of Formula (XXIV):

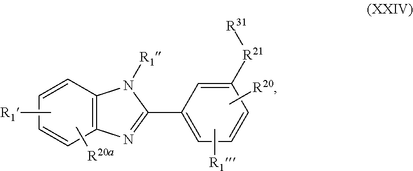

or a salt thereof, wherein:

each R²⁰ and R^{20a} is independently selected from H or a solubilizing group and at least one of R²⁰ and R^{20a} is a solubilizing group;

each R₁′, R₁″ and R₁′″ is independently selected from H or optionally substituted C₁-C₃ straight or branched alkyl;

R²¹ is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R′₁—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R′₁—C(O)—NR₁′—, —NR₁′—C(O)—CR₁′═CR₁′—CR₁′R₁′—, —NR₁′—C(═N—CN)—NR₁′—, —NR₁′—C(O)—CR₁′R′₁—O—, —NR₁′—C(O)—CR₁′R₁′—CR₁′R₁′—O—, —NR₁′—S(O)₂—CR₁′R′₁—, —NR₁′—S(O)₂—CR₁′R₁′—CR₁′R₁′—, or —NR₁′—C(O)—CR₁′R₁′—, wherein R²³ is an optionally substituted C1-C3 straight or branched alkyl; and

R³¹ is selected from an optionally substituted monocyclic or bicyclic aryl, or an optionally substituted monocyclic or bicyclic heteroaryl.

In one embodiment, the methods disclosed herein utilize administration of a sirtuin-modulating compound of Formula (XXV):

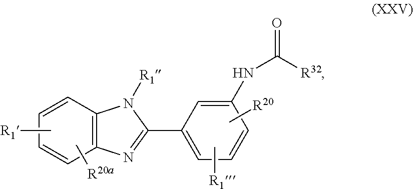

or a salt thereof, wherein:

each R²⁰ and R^{20a} is independently selected from H or a solubilizing group, wherein at least one of R²⁰ and R^{20a} is a solubilizing group;

each R₁′, R₁″ and R₁′″ is independently selected from H or optionally substituted C₁-C₃ straight or branched alkyl; and

R³² is an optionally substituted phenyl.

In one embodiment, the methods disclosed herein utilize administration of a sirtuin-modulating compound of Formula (XXVI):

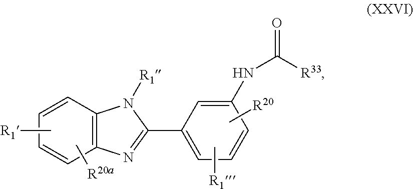

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl; and

R³³ is selected from an optionally substituted heteroaryl or an optionally substituted bicyclic aryl, with the provisos that:

when each of R₁′ and R₁′″ is hydrogen or methyl and each of R₁″, R₂₀ and R₂₀ₐ is hydrogen, R³³ is not 5,6,7,8-tetrahydronaphthyl, unsubstituted benzofuryl, unsubstituted benzothiazolyl, chloro- or nitro-substituted benzothienyl, unsubstituted furyl, phenyl-, bromo- or nitro-substituted furyl, dimethyl-substituted isoxazolyl, unsubstituted naphthyl, 5-bromonaphthyl, 4-methylnaphthyl, 1- or 3-methoxynaphthyl, azo-substituted naphthyl, unsubstituted pyrazinyl, S-methyl-substituted pyridyl, unsubstituted pyridyl, thienyl- or phenyl-substituted quinolinyl, chloro-, bromo- or nitro-substituted thienyl, unsubstituted thienyl, or

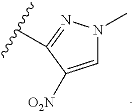

In one embodiment, the methods disclosed herein utilize administration of a sirtuin-modulating compound of Formula (XXVI):

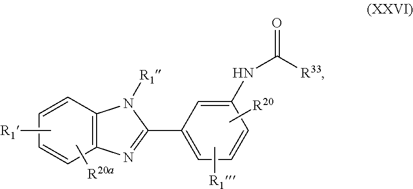

or a salt thereof, wherein:

each R²⁰ and R^{20a} is independently selected from H or a solubilizing group, wherein at least one of R²⁰ or R^{20a} is a solubilizing group;

each R₁′, R₁″ and R₁′″ is independently selected from H or optionally substituted C₁-C₃ straight or branched alkyl; and

R³³ is selected from an optionally substituted heteroaryl or an optionally substituted bicyclic aryl.

In one embodiment, the methods disclosed herein utilize administration of a sirtuin-modulating compound of Formula (XXVII):

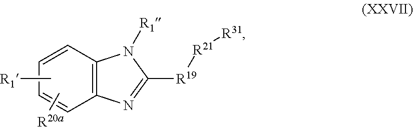

or a salt thereof, wherein:
each R²⁰ and R^{20a} is independently selected from H or a solubilizing group;
each R₁′ and R₁″ is independently selected from H or optionally substituted C₁-C₃ straight or branched alkyl;

R¹⁹ is selected from:

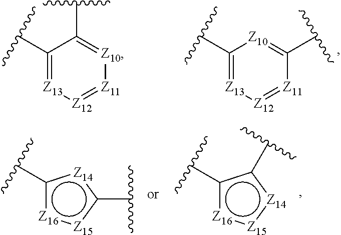

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and
R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂₋₅—NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R′₁—, or —NR₁′—C(O)—CR₁′R₁′—; and

R³¹ is selected from an optionally substituted monocyclic or bicyclic aryl, or an optionally substituted monocyclic or bicyclic heteroaryl,
provided that when R²¹ is —NH—C(O)— and R¹⁹ is

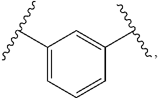

R³¹ is not unsubstituted pyridyl, 2,6-dimethoxyphenyl, 3,4,5-trimethoxyphenyl or unsubstituted furyl.

In a particular aspect, the invention provides sirtuin-modulating compounds of Structural Formula (XXVII):

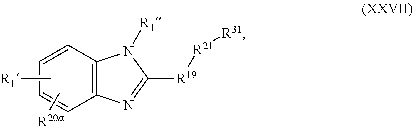

or a salt thereof, wherein:
each R²⁰ and R^{20a} is independently selected from H or a solubilizing group;
each R₁′ and R₁″ is independently selected from H or optionally substituted C₁-C₃ straight or branched alkyl;

R¹⁹ is selected from:

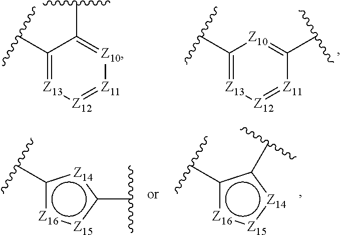

wherein:

each Z₁₀, Z₁₁, Z₁₂ and Z₁₃ is independently selected from N, CR²⁰, or CR₁′; and

each Z₁₄, Z₁₅ and Z₁₆ is independently selected from N, NR₁′, S, O, CR²⁰, or CR₁′, wherein:

zero to two of Z₁₀, Z₁₁, Z₁₂ or Z₁₃ are N;

at least one of Z₁₄, Z₁₅ and Z₁₆ is N, NR₁′, S or O;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group;

zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R′₁—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R′₁₋₅—NR₁′—C(O)—CR₁′═CR₁′—, —NR₁′—S(O)₂—NR₁′—, —NR₁′—C(O)—NR₁′—S(O)₂—, —NR₁′—CR₁′R′₁—C(O)—NR₁′—, —CR₁′R′₁—C(O)—NR₁′—, —NR₁′—C(O)—CR₁′═CR₁′—CR₁′R₁′—, —NR₁′—C(═N—CN)—NR₁′—, —NR₁′—C(O)—CR₁′R′₁—O—, —NR₁′—C(O)—CR₁′R₁′—CR₁′R₁′—O—, —NR₁′—S(O)₂—CR₁′R′₁—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—, R¹⁹ is not pyrazolyl;

when R²¹ is —NH—, and R¹⁹ is thiazolyl, R³¹ is not optionally substituted phenyl or optionally substituted pyridyl;

when R²¹ is —NH—C(O)—CH₂—, and R¹⁹ is pyrazolyl, R³¹ is not unsubstituted indolyl or unsubstituted phenyl;

when R²¹ is —NH—C(O)—CH₂—, and R¹⁹ is

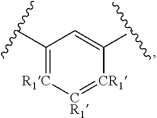

R³¹ is not 2-methylphenyl or 3,4-dimethoxyphenyl;

when R²¹ is —NH—C(O)—CH═CH—, and R¹⁹ is

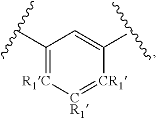

R³¹ is not 2-chlorophenyl;

when R²¹ is —NH—C(O)—NH—, and R¹⁹ is pyrazolyl, R³¹ is not unsubstituted isoxazolyl, unsubstituted naphthyl, unsubstituted phenyl, 2,6-difluorophenyl, 2,5-dimethylphenyl, 3,4-dichlorophenyl, or 4-chlorophenyl;

when R²¹ is —NH—C(O)—NH—, and R¹⁹ is

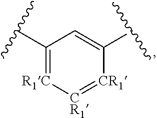

R³¹ is not unsubstituted benzimidazolyl;

when R²¹ is —NH—, and R¹⁹ is pyrazolyl, R³¹ is not unsubstituted pyridyl;

when R^{20a} is a solubilizing group, R¹⁹ is 1-methylpyrrolyl and R²¹ is —NH—C(O)—, R³¹ is not unsubstituted phenyl, unsubstituted furyl, unsubstituted pyrrolyl, unsubstituted pyrazolyl, unsubstituted isoquinolinyl, unsubstituted benzothienyl, chloro-substituted benzothienyl, 2-fluoro-4-chlorophenyl or phenyl singly substituted with a solubilizing group;

when R^{20a} is a solubilizing group, R¹⁹ is thienyl and R²¹ is —NH—C(O)—, R³¹ is not unsubstituted phenyl;

when R^{20a} is a solubilizing group, R¹⁹ is methylimidazolyl and R²¹ is —NH—C(O)—, R³¹ is not 1-methyl-4-(1,1-dimethylethyloxycarbonylamino)pyrrol-2-yl or phenyl singly substituted with a solubilizing group;

when R²¹ is —NH— and R¹⁹ is pyridyl, oxadiazolyl or thiadiazolyl, R³¹ is not unsubstituted phenyl, 3-methoxyphenyl or 4-methoxyphenyl;

when R²¹ is —NH—C(O)— and R¹⁹ is thiazolyl or pyrimidinyl, R³¹ is not unsubstituted phenyl;

when R²¹ is —NH—C(O)— and R¹⁹ is

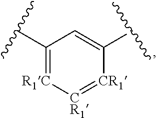

R³¹ is not unsubstituted pyridyl, unsubstituted thienyl, unsubstituted phenyl, 2-methylphenyl, 4-fluorophenyl, 4-methoxyphenyl, 4-methylphenyl, 3,4-dioxyethylenephenyl, 3-acetylamino-4-methylphenyl, 3-[(6-amino-1-oxohexyl)amino]-4-methylphenyl, 3-amino-4-methylphenyl, 2,6-dimethoxyphenyl, 3,5-dimethoxyphenyl, 3-halo-4-methoxyphenyl, 3-nitro-4-methylphenyl, 4-propoxyphenyl, 3,4,5-trimethoxyphenyl or unsubstituted furyl;

when R²¹ is —NH—C(O)— and R¹⁹ is

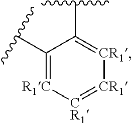

R³¹ is not 3,5-dinitrophenyl, 4-butoxyphenyl,

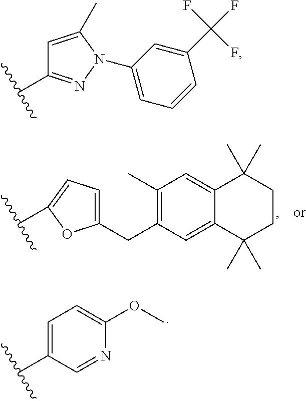

In a more particular embodiment, the invention provides sirtuin-modulating compounds of Structural Formula (XXVII):

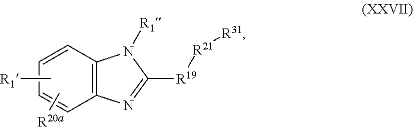

or a salt thereof, wherein:

each R²⁰ and R^{20a} is independently selected from H or a solubilizing group;

each R₁′ and R₁″ is independently selected from H or optionally substituted C₁-C₃ straight or branched alkyl;

R¹⁹ is selected from:

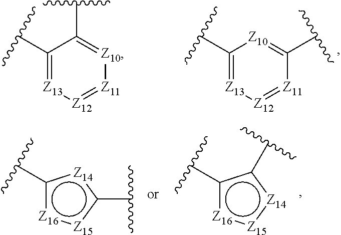

wherein:

each Z₁₀, Z₁₁, Z₁₂ and Z₁₃ is independently selected from N, CR²⁰, or CR₁′; and

each Z₁₄, Z₁₅ and Z₁₆ is independently selected from N, NR₁′, S, O, CR²⁰, or CR₁′, wherein:

one to two of Z₁₀, Z₁₁, Z₁₂ or Z₁₃ are N;

at least one of Z₁₄, Z₁₅ and Z₁₆ is N, NR₁′, S or O;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group;

zero to one R₁′″ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R′₁—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R′₁—C(O)—NR₁′—, —NR₁′—C(O)—CR₁′═CR₁′—CR₁′R₁′—, —NR₁′—C(═N—CN)—NR₁′—, —NR₁′—C(O)—CR₁′R′₁—O—, —NR₁′—C(O)—CR₁′R₁′—CR₁′R₁′—O—, —NR₁′—S(O)₂—CR₁′R′₁—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—, R¹⁹ is not pyrazolyl;

when R²¹ is —NH—C(O)—CH₂—, and R¹⁹ is pyrazolyl, R³¹ is not unsubstituted indolyl or unsubstituted phenyl;

when R²¹ is —NH—C(O)—NH—, and R¹⁹ is pyrazolyl, R³¹ is not unsubstituted isoxazolyl, unsubstituted naphthyl, unsubstituted phenyl, 2,6-difluorophenyl; 2,5-dimethylphenyl; 3,4-dichlorophenyl; or 4-chlorophenyl;

when R^{20a} is a solubilizing group, R¹⁹ is 1-methylpyrrolyl and R²¹ is —NH—C(O)—, R³¹ is not unsubstituted phenyl; unsubstituted furyl; unsubstituted pyrrolyl; unsubstituted pyrazolyl; unsubstituted isoquinolinyl; unsubstituted benzothienyl; chloro-substituted benzothienyl; 2-fluoro-4-chlorophenyl or phenyl singly substituted with a solubilizing group;

when R^{20a} is a solubilizing group, R¹⁹ is thienyl and R²¹ is —NH—C(O)—, R³¹ is not unsubstituted phenyl;

when R^{20a} is a solubilizing group, R¹⁹ is methylimidazolyl and R²¹ is —NH—C(O)—, R³¹ is not 1-methyl-4-(1,1-dimethylethyloxycarbonylamino)pyrrol-2-yl or phenyl singly substituted with a solubilizing group; and

when R²¹ is —NH—C(O)— and R¹⁹ is thiazolyl or pyrimidinyl, R³¹ is not unsubstituted phenyl.

In one embodiment, the methods disclosed herein utilize administration of a sirtuin-modulating compound of Formula (XXVIII):

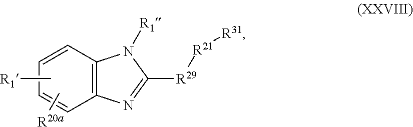

or a salt thereof, wherein:

each R²⁰ and R^{20a} is independently selected from H or a solubilizing group;

each R₁′ and R₁″ is independently selected from H or optionally substituted C₁-C₃ straight or branched alkyl;

R²⁹ is selected from:

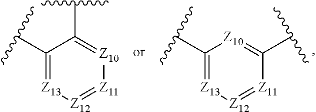

wherein:

each Z₁₀, Z₁₁, Z₁₂ and Z₁₃ is independently selected from N, CR²⁰, or CR₁′, wherein one of Z₁₀, Z₁₁, Z₁₂ or Z₁₃ is N; and

zero to one R²⁰ is a solubilizing group;

zero to one R₁′″ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R′₁—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R′₁—C(O)—NR₁′—, —NR₁′—C(O)—CR₁′═CR₁′—CR₁′R₁′—, —NR₁′—C(═N—CN)—NR₁′—, —NR₁′—C(O)—CR₁′R′₁—O—, —NR₁′—C(O)—CR₁′R₁′—CR₁′R₁′—O—, —NR₁′—S(O)₂—CR₁′R′₁—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl.

The methods disclosed herein may also utilize pharmaceutical compositions comprising one or more compounds of Formulas (I)-(XXVIII) or a salt, prodrug or metabolite thereof.

### 7. Kits and Screening Assays

Provided herein are kits that may be used to determine the presence or absence of a T373N or L107X Sirt1 polymorphic variant. Such kits may be used to diagnose, or predict a subject's susceptibility to, a Sirt1 mediated disease or disorder. This information could then be used, for example, to optimize treatment with a sirtuin modulating compound for subjects having a T373N or L107X Sirt1 polymorphic variant.

In preferred embodiments, the kit comprises a probe or primer which is capable of hybridizing to a T373N polymorphic variant of a Sirt1 gene thereby determining whether the Sirt1 gene contains a polymorphic variant that is associated with a risk of having or developing a Sirt1 mediated disease or disorder. The kit may further comprise instructions for use in diagnosing a subject as having, or having a predisposition, towards developing a Sirt1 mediated disease or disorder. The probe or primers of the kit can be a probe or primer that binds to at least a portion of SEQ ID NO: 1 comprising nucleotide residue 373, or a sequence complementary thereto.

Kits for amplifying a region of a gene comprising a T373N variant of Sirt1 may comprise one, two or more primers. Exemplary primers are provided in Example 1.

In an exemplary embodiment, a kit may comprise a microarray suitable for detection of a T373N Sirt1 polymorphic variant. Examples of such microarrays are described further herein above.

In other embodiments, the kits provided herein may comprise an antibody that is capable of specifically recognizing a L107X Sirt1 polymorphic variant. In an exemplary embodiment, the kit may include a panel of antibodies able to specifically bind to a variety of polypeptide variants of Sirt1. The kits may further comprise additional components such as substrates for an enzymatic reaction. The antibodies may be used for research, diagnostic, and/or therapeutic purposes.

In yet other embodiments, the kits provided herein may comprise reagents for detecting deacetylase activity of a Sirt1 polypeptide, such as, a wild-type Sirt1 polypeptide or a L107X Sirt1 polymorphic variant. For example, the kits may comprise a Sirt1 substrate, buffers, detection reagents, etc.

In other embodiments, methods for identifying sirtuin modulating compounds are provided. The methods may involve for example, correlating the presence or absence of a T373N or L107X Sirt1 polymorphic variant with the activity or efficacy of a sirtuin modulating compound. Such methods may be carried out using in vitro or in vivo methods for determining Sirt1 activity and/or efficacy.

Intact cells or whole animals expressing a T373N or L107X Sirt1 polymorphic variant, can be used in screening methods to identify candidate drugs. For example, a permanent cell line may be established from an individual exhibiting a T373N or L107X Sirt1 polymorphic variant. Alternatively, cells (including without limitation mammalian, insect, yeast, or bacterial cells) may be programmed to express a gene comprising a T373N Sirt1 nucleic acid variant or encoding a L107X Sirt1 polypeptide variant by introduction of appropriate DNA into the cells. Identification of candidate sirtuin modulating compounds can be achieved using any suitable Sirt1 deacetylase assay. A variety of assays are known in the art or are commercially available. Exemplary sirtuin deacetylase assays are described herein below. Such assays may include without limitation (i) assays that measure selective binding of test compounds to L107X polypeptide variants of Sirt1; and (ii) assays that measure the ability of a test compound to modify (i.e., inhibit or enhance) a measurable activity or function of a L107X polypeptide variant of Sirt1.

In other embodiments, transgenic animals are created in which (i) a human Sirt1 gene having a T373N or L107X Sirt1 polymorphic variant is stably inserted into the genome of the transgenic animal; and/or (ii) the endogenous Sirt1 gene may be inactivated and replaced with the human Sirt1 variant sequence. See, e.g., Coffman, Semin. Nephrol. 17:404, 1997; Esther et al., Lab. Invest. 74:953, 1996; Murakami et al., Blood Press. Suppl. 2:36, 1996. Such animals can be treated with candidate compounds and monitored, for example, for one or more clinical markers of disease, expression levels (mRNA and/or protein) of Sirt1, activity of Sirt1, etc.

### EXEMPLIFICATION

The invention now being generally described, it will be more readily understood by reference to the following examples which are included merely for purposes of illustration of certain aspects and embodiments of the present invention, and are not intended to limit the invention in any way.

### Example 1

### Genetic Analysis of Sirt1

Type 1 diabetes is diagnosed in lean and young individuals displaying hyperglycemia, markers of auto-immune destruction of the insulin producing β-cell, and rapidly becoming dependent on insulin for survival (Diagnosis and classification of diabetes mellitus. Diabetes Care 31 Suppl 1, S55-60 (2008)). Type 2 diabetes is associated with ageing and is characterised by hyperglycemia due to insulin resistance and progressive β-cell dysfunction. Sirt1 is a protein deacetylase implicated in ageing and in the beneficial effects of calorie restriction. Administration of Sirt1 activators are proposed to prevent β-cell secretory failure and insulin resistance in patients with type 2 diabetes (Westphal, C. H., Dipp, M. A. & Guarente, L. A therapeutic role for sirtuins in diseases of aging? Trends Biochem Sci 32, 555-60 (2007); Milne, J. C. et al. Small molecule activators of Sirt1 as therapeutics for the treatment of type 2 diabetes. Nature 450, 712-6 (2007)). Although increasing evidence points to a common denominator in the pathogenesis of type 1 and 2 diabetes, the conditions are considered to be two separate entities with different predisposing factors (Donath, M. Y. & Ehses, J. A. Type 1, type 1.5, and type 2 diabetes: NOD the diabetes we thought it was. Proc Natl Acad Sci USA 103, 12217-8 (2006)).

The classification of diabetes distinguishes between type 1 diabetes, characterized by autoimmune β-cell destruction, and type 2 diabetes, caused by a relative insulin deficiency in the face of insulin resistance (Diagnosis and classification of diabetes mellitus. Diabetes Care 31 Suppl 1, S55-60 (2008)). Increasing clinical evidence is emerging that points to some overlap between these two diabetic conditions (Donath, M. Y. & Ehses, J. A. Type 1, type 1.5, and type 2 diabetes: NOD the diabetes we thought it was. Proc Natl Acad Sci USA 103, 12217-8 (2006)). For example, obesity, which is associated with insulin resistance and type 2 diabetes, correlates with the recent increased incidence of type 1 diabetes (Kibirige, M., Metcalf, B., Renuka, R. & Wilkin, T. J. Testing the accelerator hypothesis: the relationship between body mass and age at diagnosis of type 1 diabetes. Diabetes Care 26, 2865-70 (2003); Hypponen, E., Virtanen, S. M., Kenward, M. G., Knip, M. & Akerblom, H. K. Obesity, increased linear growth, and risk of type 1 diabetes in children. Diabetes Care 23, 1755-60 (2000); Libman, I. M., Pietropaolo, M., Arslanian, S. A., LaPorte, R. E. & Becker, D. J. Changing prevalence of overweight children and adolescents at onset of insulin-treated diabetes. Diabetes Care 26, 2871-5 (2003)). Furthermore, inflammatory mediators have been implicated in insulin resistance and in the failure of the β-cell of type 1 and 2 diabetes (Hotamisligil, G. S., Shargill, N. S. & Spiegelman, B. M. Adipose expression of tumor necrosis factor-alpha: direct role in obesity-linked insulin resistance. Science 259, 87-91 (1993); Donath, M. Y., Storling, J., Berchtold, L. A., Billestrup, N. & Mandrup-Poulsen, T. Cytokines and beta-Cell Biology: from Concept to Clinical Translation. Endocr Rev (2007)). Although multiple gene defects are associated with overall susceptibly to type 1 and type 2 diabetes, single gene defects leading to diabetes have been identified only in a small subgroup of patients. These monogenic forms are termed maturity-onset diabetes of the young and are characterized by onset of hyperglycemia at an early age due to impaired insulin secretion (Diagnosis and classification of diabetes mellitus. Diabetes Care 31 Suppl 1, S55-60 (2008)). Abnormalities at six genetic loci on different chromosomes have been identified, inherited in an autosomal dominant pattern. However, none of them is associated with auto-immune destruction of the β-cell and/or abnormal insulin sensitivity.

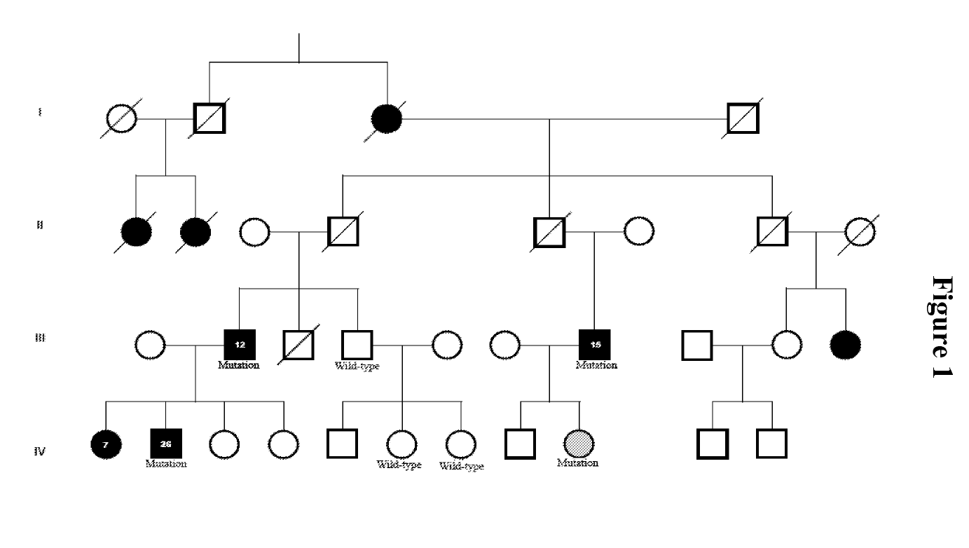

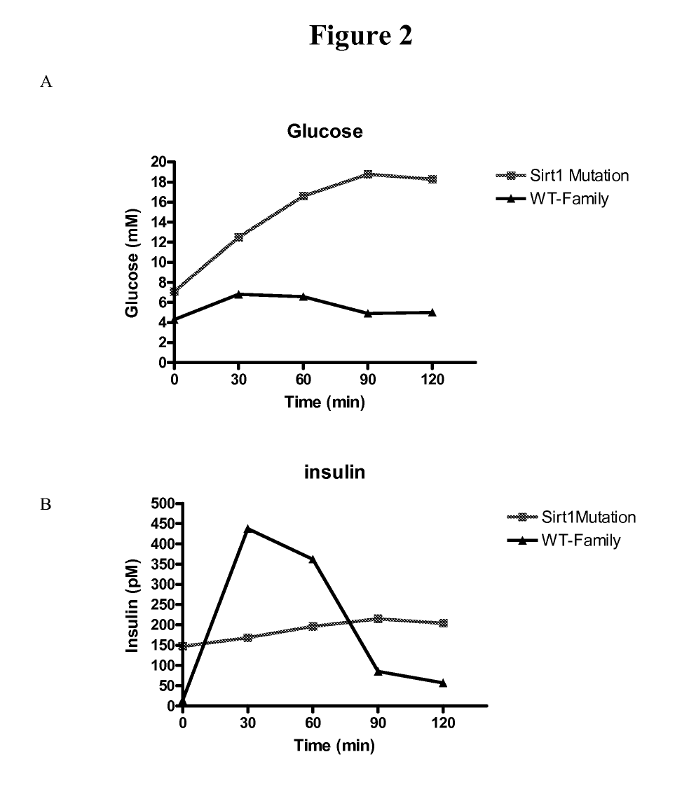

Type 1 diabetes was diagnosed in a 26-year-old Ashkenazy Jewish man on the basis of hyperglycaemia, lean body mass index of 21.5 Kg/m², signs of β-cell autoimmunity (auto-antibodies to glutamic acid decarboxylase 1150 U/L, islet-cell autoantibody-2 3.0 U/L) and rapid insulin dependence. His father and sister were also diagnosed with type 1 diabetes at the ages of 12 and 7 years, respectively, and several other family members were also affected (FIG. 1). The pattern of the affected family members were compatible with an autosomal dominant mutation. To further characterize the disease, an oral glucose-tolerance test was performed 10 months after onset of diabetes. As expected β-cell function was severely impaired with almost no increase in insulin release following a stimulation by an oral glucose load (FIG. 2). However, basal insulin levels were surprisingly high compared to a control 20-year-old non-affected male family member. Interestingly, an intravenous injection of glucose combined with glucagon and arginine (Larsen, C. M. et al. Interleukin-1-receptor antagonist in type 2 diabetes mellitus. N Engl J Med 356, 1517-26 (2007)) led to strong increase in serum insulin level up to 603 pmol/l along with 2460 μmol/l c-peptide. This suggests that the exocytotic machinery is functional and indicates a defect in metabolism-secretion coupling. The patient also exhibited unexpectedly severe insulin resistance in the same range as patients with type 2 diabetes (Larsen 2007, supra). The clinical relevance of the insulin resistance was demonstrated by 6 months of therapy with metformin. During the period of metformin administration, the patient did not require basal treatment with insulin in order to maintain normal glycaemia.

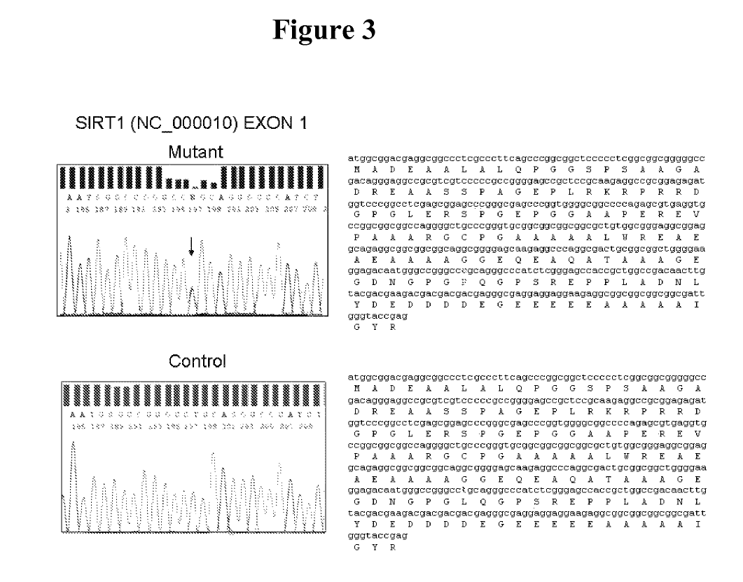

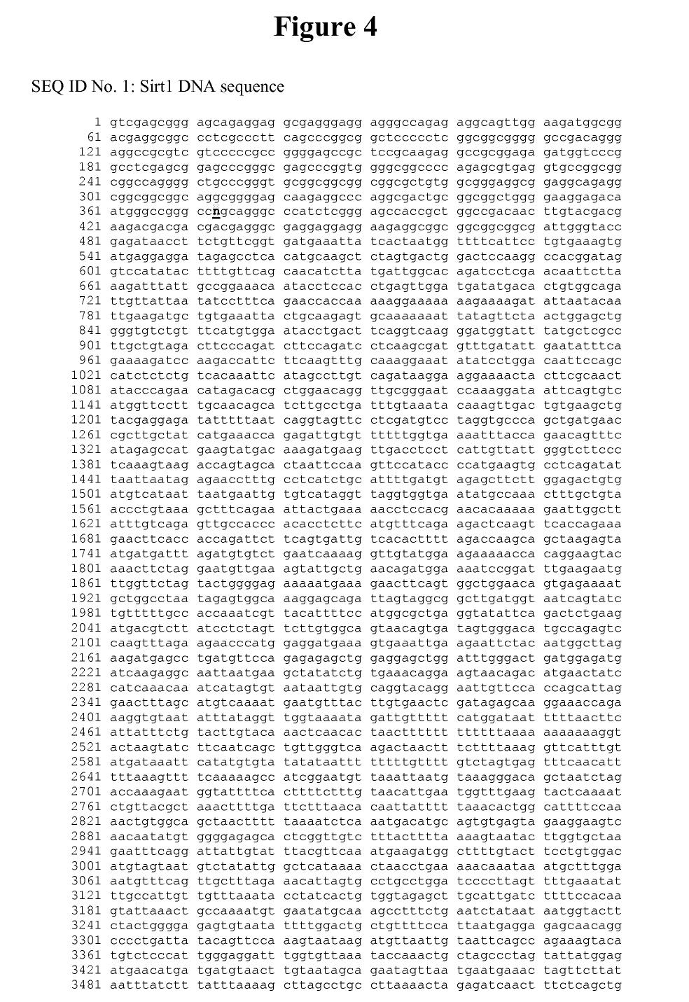

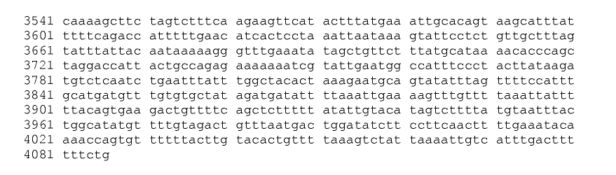

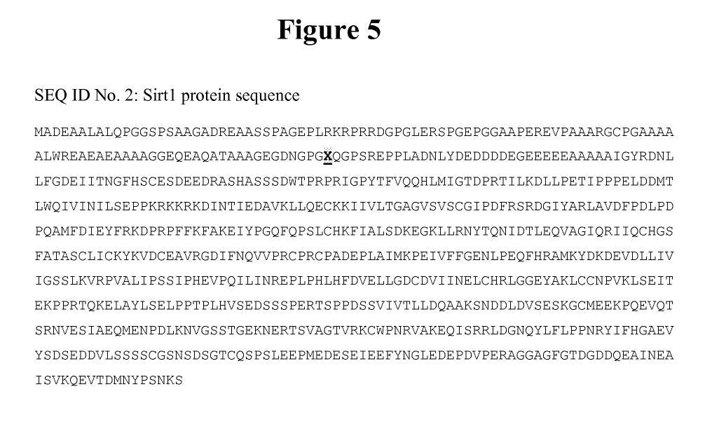

The patients were screened for mutations in candidate genes that would explain a combined dysfunction of insulin secretion due to a defect in metabolism-secretion coupling along with insulin resistance. Sirt1 is preferentially expressed in β-cells and regulates insulin secretion. Indeed, Sirt1 knockout mice have increased UCP2 expression with consecutive failure of β-cells to increase ATP levels after glucose stimulation (Bordone, L. et al. Sirt1 regulates insulin secretion by repressing UCP2 in pancreatic beta cells. PLoS Biol 4, e31 (2006)). Conversely, targeted over-expression of Sirt1 in the β-cell enhances insulin secretion (Moynihan, K. A. et al. Increased dosage of mammalian Sir2 in pancreatic beta cells enhances glucose-stimulated insulin secretion in mice. Cell Metab 2, 105-17 (2005)). In parallel, Sirt1 is directly involved in insulin sensitivity (Sun, C. et al. Sirt1 improves insulin sensitivity under insulin-resistant conditions by repressing PTP1B. Cell Metab 6, 307-19 (2007)). Direct sequencing of the Sirt1 gene revealed that the affected individuals were heterozygous for a T to C mutation in exon 1 (c.T373C), leading to a Leucine 107 to Proline mutation in the Sirt1 protein (FIG. 3). Furthermore, the presence of a mutation in three other genes known to have such a dual effect in animal models, TCF7L2, IRS2 and SOCS2 (Lyssenko, V. et al. Mechanisms by which common variants in the TCF7L2 gene increase risk of type 2 diabetes. J Clin Invest 117, 2155-63 (2007); Rhodes, C. J. Type 2 diabetes-a matter of beta-cell life and death? Science 307, 380-4 (2005); Ronn, S. G., Billestrup, N. & Mandrup-Poulsen, T. Diabetes and suppressors of cytokine signaling proteins. Diabetes 56, 541-8 (2007)), was excluded. FIG. 2 shows that a representative non-affected family member was healthy with normal insulin secretion and action (FIG. 2). Every affected family member was diagnosed for type 1 diabetes except for an 18 year old woman with a severe ulcerative colitis which manifested at the age of 16 year and requested maintenance therapy with azathioprine. Increased levels of anti-nuclear antibodies (titer: 1:640) and ANCA (titer: 1:160) reflected the autoimmune character of the disease. Metabolic evaluation under ongoing immunosuppression revealed no abnormality.

### Methods Summary

After obtaining informed consent, genomic DNA was extracted from peripheral blood leukocytes using the QIAGEN DNA blood and cell culture kit (QIAGEN GmbH, Hilden, Germany) and used to perform polymerase chain reaction (PCR) exonic amplifications. DNA from 50 unrelated individuals (30 Caucasian, 8 of whom from Turkey; 10 Asian; 10 Blacks, 2 of whom from Central America and 2 Hispanic) was used as controls for polymorphisms. The coding region of Sirt1 (NC_{—}00001.0) was also analyzed and the PCR products were sequenced using the Big Dye Terminator Cycle Sequencing Kit and analyzed on an ABI Prism 310 Genetic Analyzer (Applied Biosystems). Primer sequences are displayed in the table below. The coding regions of TCF7L2 (Transcription factor 7-like 2, NC_{—}000010.9), IRS2 (Insulin Receptor Substrate 2, NC_{—}000013.9) and SOCS2 (Suppressor of cytokine signaling 2, NC_{—}000012.10) were also examined.

| | | | SIZE | | SEQ ID |
|---|---|---|---|---|---|
| EXON | PRIMER NAME | SEQUENCE (5′-3′) | (BP) | Opt T (max) | NO |
| 1 | Sirt1Ex1-5′ | GCGGGAGCAGAGGAGGCGAGGGA | 608 | 71.1 (72) | 4 |
| | Sirt1Ex1-3′ | GGGCCGGGGTGGGGAGGGGAACG | | | 5 |
| | Sirt1Ex1-dir | GGCAGTTGGAAGATGGCGGACGA | | | 6 |
| | Sirt1Ex1-dir2 | GGAGCAAGAGGCCCAGGCGACTG | | | 7 |
| | Sirt1Ex1-rev | GTACCCAATCGCCGCCGCCGCCG | | | 8 |
| 2 | Sirt1Ex2-5′ | ACACACGCCGTGTAAATGTTAAA | 521 | 57.6 (72) | 9 |
| | Sirt1Ex2-3′ | CGGATCACCTGAGGTCGGGAGTT | | | 10 |
| | Sirt1Ex2-dir | GCCGTGTAAATGTTAAATGCTGT | | | 11 |
| | Sirt1Ex2-rev | ACTCCAGCCTCCGCAACAAGAGC | | | 12 |
| 3 | Sirt1Ex3-5′ | GTCTTCCCAACTCTTCATTAGAT | 566 | 52.3 (68.8) | 13 |
| | Sirt1Ex3-3′ | AGTTCCCAAACATCCATTATCAT | | | 14 |
| | Sirt1Ex3-dir | CTTAAAGATTTATTGCCGGAAAC | | | 15 |
| | Sirt1Ex3-rev | GTTTCCGGCAATAAATCTTAAG | | | 16 |
| 4 | Sirt1Ex4-5′ | CTGAAATAAGGGTAGGGTTGTAT | 439 | 52.6 (68.7) | 17 |
| | Sirt1Ex4-3′ | ATCTTACTCCGCCACAGTAGCAG | | | 18 |
| | Sirt1Ex4-dir | GATGGTATTTATGCTCGCCTTGC | | | 19 |
| | Sirt1Ex4-rev | TTACTCCGCCACAGTGCAGCAT | | | 20 |
| | Sirt1Ex4-rev2 | AGTCTACAGCAAGGCGAGCATAA | | | 21 |
| 5 | Sirt1Ex5-5′ | AGAGATGTTTATGGGCCGACTTT | 585 | 52.9 (70.6) | 22 |
| | Sirt1Ex5-3′ | TTAACTGTCAAGGCCAATACTTC | | | 23 |
| | Sirt1Ex5-dir | CCTTTGGATTCCCGCAACCTGTT | | | 24 |
| | Sirt1Ex5-rev | AACAGGTTGCGGGAATCCAAAGG | | | 25 |
| 6 | Sirt1Ex6-5′ | CAAAACAAAAATCCTTAAACCC | 448 | 51.1 (65.1) | 26 |
| | Sirt1Ex6-3′ | ACATTTTATGTTCGGCTTAGATA | | | 27 |
| | Sirt1Ex6-dir | AAAGTTGACTGTGAAGCTGTACG | | | 28 |
| | Sirt1Ex6-rev | CGTACAGCTTCACAGTCAACTTT | | | 29 |
| 7 | Sirt1Ex7-5′ | GACTATTTCTAACTTGGGCTTAC | 322 | 52.6 (66.6) | 30 |
| | Sirt1Ex7-3′ | GTCTATTATACAGACCCACAACC | | | 31 |
| | Sirt1Ex7-dir | GGGCTTACTCTTTGCTTCTCTAC | | | 32 |
| | Sirt1Ex7-rev | TATAGAAATGTTCTCCAAAAACC | | | 33 |
| 8 | Sirt1Ex8-5′ | CCCTTGTTGGATTTTTGCATAAT | 761 | 52.9 (66.5) | 34 |
| | Sirt1Ex8-3′ | AATCAACTACATAAAGCTACCCT | | | 35 |
| | Sirt1Ex8-dir | TTATTTTTCACCCTATTTAGGTT | | | 36 |
| | Sirt1Ex8-rev | CGATTAACCTGCCGAAATAGCTA | | | 37 |
| | Sirt1Ex8rev2 | GAACCAACATTCTTCAAATCCGG | | | 38 |
| 9 | Sirt1Ex9-5′ | AAGGCAGAGCTGGAACCCACACT | 671 | 53.7 (70.7) | 39 |
| (CDS) | Sirt1Ex9-3′ | CCAACAGCTGATTGAAGATACTT | | | 40 |
| | Sirt1Ex9-dir | TGCCAGAGTCCAAGTTTAGAAGA | | | 41 |
| | Sirt1Ex9-rev | TCTTCTAAACTTGGACTCTGGCA | | | 42 |

Two-hour oral glucose-tolerance tests were performed as described (Larsen (2007), supra). Measurements of plasma glucose, proinsulin, insulin, and C-peptide followed by intravenous injection of 0.3 g of glucose per kilogram of body weight, 0.5 mg of glucagon, and 5 g of arginine, euglycemic—hyperinsulinemic clamp studies and muscle biopsies were performed.

### Example 2

### Analysis of Sirtuin Activity

The following example describes methods for the identification and characterization of Sirt1 activators. Human Sirt1 is expressed from the pSirt1FL vector which places expression under the control of the T7 promoter. The protein is expressed in E. coli BL21(DE3)Star as an N-terminal fusion to a hexa-histidine affinity tag. The expressed protein is purified by Ni²⁺-chelate chromatography. The eluted protein is then purified by size exclusion chromatography followed by ion exchange. The resulting protein is typically >95% pure as assessed by SDS-PAGE analysis. The mass spectrometry based assay utilizes a peptide having 20 amino acid residues as follows: Ac-Glu-Glu-Lys(Biotin)-Gly-Gln-Ser-Thr-Ser-Ser-His-Ser-Lys(Ac)-Nle-Ser-Thr-Glu-Gly-Lys(5TMR)-Glu-Glu-NH2 (SEQ ID NO: 3) wherein K(Ac) is an acetylated lysine residue and Nle is a norleucine. The peptide is labeled with the fluorophore 5TMR (excitation 540 nm/emission 580 nm) at the C-terminus for use in the FP assay described above. The sequence of the peptide substrate is based on p53 with several modifications.

The mass spectrometry assay is conducted as follows: 0.5 μM peptide substrate and 120 μM βNAD⁺ is incubated with 10 nM Sirt1 for 25 minutes at 25° C. in a reaction buffer (50 mM Tris-acetate pH 8, 137 mM NaCl, 2.7 mM KCl, 1 mM MgCl₂, 5 mM DTT, 0.05% BSA). Test compounds are added to the reaction or vehicle control, DMSO. After the incubation with Sirt1, 10% formic acid is added to stop the reaction. Determination of the mass of the substrate peptide allows for precise determination of the degree of acetylation (i.e. starting material) as compared to deacetylated peptide (product).

### Example 3

### Mass Spectrometry Analysis of Sirtuin Activity

The following example describes an alternative mass spec based assay for determination of Sirt1 deacetylase activity. Instead of relying on purified or recombinant enzyme, the reaction utilizes endogenous Sirt1 enzyme from cell or tissue extracts. The endogenous Sirt1 may have leucine or an amino acid other than leucine (such as proline) at position 107. This assay allows for the determination of endogenous sirtuin activity. For tissues, cells or samples of human origin, the Sirt1 haplotype can also be determined and correlated with Sirt1 enzymatic activity. The cells or tissues can be pretreated with Sirt1 modulators or other control compounds either following isolation or following pharmacological intervention in vivo. Alternatively, this measurement of endogenous sirtuin activity can be measured in various clinical samples following physiological manipulation (diet, exercise, age, disease progression, etc.) or following pharmacological intervention including studies designed to study dose responsiveness and escalation, vehicle or placebo control, dosing regimen, drug combination and synergy, etc.

This example describes the procedure for isolating viable (living) white blood cells (WBC) (also called “Peripheral Blood Mononuclear Cells”) from whole blood. The isolated WBC can then be used to determine Sirt1 gene haplotype as described herein, measure citrate synthase (CS, EC 4.1.3.7) activity or mitochondrial DNA (mtDNA) content. These latter two parameters represent markers of mitochondrial content in WBC. Changes in WBC CS activity or mtDNA over time in a given individual reflect changes in WBC mitochondrial oxidative capacity. Depending on the treatment (e.g. activation of mitochondriogenesis through a factor that is expressed in all tissues), changes in WBC mitochondrial oxidative capacity can reflect changes in mitochondrial oxidative capacity in other tissues (e.g. skeletal muscle, white adipose tissue).

This procedure is based on approximately 6 ml of whole blood (Vacutainer format). This is the content of a standard tube (Becton Dickinson Vacutainer™ CPT™ Cell Preparation Tubes with Sodium Heparin, cat. # 362753). Mix the blood before centrifugation by 10 times gently inverting the tube up and down. Centrifuge the CPT-tubes 20 minutes at 1700 RCF (3100 RPM) at room temperature (18-25° C.) with the brake off. Open the CPT tube and remove the plasma (4 ml) without disturbing the cell phase. Store the plasma if necessary. Remove the cell phase (ca. 2 ml, containing WBC, platelets and some plasma) with a plastic Pasteur (transfer) pipette and transfer this phase to a 15 ml conical Falcon-tube. Add phosphate buffered saline (PBS) to the cells to bring the volume up to 13 ml. Mix carefully by inverting the tube. Centrifuge the 15 ml conical tube at 300 RCF (1200 RPM) for 15 minutes at room temperature (18-25° C., no brake). Aspirate the supernatant (PBS, platelets and some plasma) without disturbing the cell pellet, and resuspend the cell pellet (WBC) in the remaining PBS (approximately 200 μl). Add PBS to the remaining cell suspension to bring the volume up to 13 ml, mix carefully by inverting the tube. Centrifuge at room temperature at 300 RCF (1200 RPM) for 15 minutes at room temperature (18-25° C., no brake). Aspirate the supernatant without disturbing the cell pellet, and resuspend the cell pellet in the remaining PBS (approximately 200 μl). Add PBS to the remaining cell suspension to bring the volume up to 10 ml, mix carefully by inverting the tube. Centrifuge at room temperature at 300 RCF (1200 RPM) for 15 minutes at room temperature (18-25° C., no brake). Aspirate the supernatant without disturbing the cell pellet. From this point keep the cells on ice.

Add 1 ml Freeze Medium without FBS(RPMI Medium 1640 with L-Glutamine; DMSO (dimethyl sulfoxide), 10% (vol:vol) final) to the remaining cell pellet and resuspend the cells gently. For some uses where plasma proteins do not interfere with the assay, e.g. for mtDNA quantification (but not for CS activity measurement), the WBC pellet can be resuspended and frozen in Freeze Medium with FBS (RPMI Medium 1640 with L-Glutamine; DMSO (dimethyl sulfoxide), 10% (vol:vol) final; FBS (Fetal Bovine Serum), heat inactivated 30 minutes at 56° C., 20% (vol:vol) final. Plasma proteins help maintain cell integrity when frozen. Once the Freeze Medium is added the cells must remain on wet ice for the remainder of the process and should be frozen as soon as possible. Transfer the cell suspension into cryovials (2 aliquots of 0.5 ml per sample). Freeze the cryovials by placing them into a −80° C. freezer. Keep the WBC samples at −80° C. until use. Six mililiters of blood gives around 10 million WBC, containing around 4 μg total RNA, 40 μg total cell proteins and 0.15 ng Sirt1 protein.

600-800 million WBC corresponding to ˜0.26 nM of Sirt1 in 20 μL of final lysate are used for a standard experiment to measure the activity of Sirt1 with five time points in triplicate for two given sets of experiments. The amount of Sirt1 in each preparation is determined initially by Western-Blot analysis using different amounts of WBC with a given Sirt1 standard (purified Sirt1, bacterially expressed).

The WBC are thawed and collected in a single 15 mL falcon tube at 4 degrees Celsius. The assay buffer consists of 10× reaction buffer, 5 mM DTT and 0.05% BSA. The reaction buffer is prepared as a 10× stock and consists of 500 mM Tris HCl pH 8.0, 1370 mM NaCl, 27 mM KCl, and 10 mM MgCl₂. The buffer is stored at room temperature. Prior to use the final assay buffer is chilled at 4 degrees Celsius. 700 μL of assay buffer is added to the collected WBC and gently mixed. Cells are sonicated on ice for 2 minutes with intervals (15 seconds sonication, 30 seconds pause) at a power output level of 1.5 with a small sonicator probe (Virsonic sonicator). The sonicated cells are centrifuged for 5 minutes at 3000 rpm and the supernatant (referred as “lysate”) is removed for further use in the activity assay.

Alternatively, lysates can be prepared from tissue, such as liver, fat or muscle. Typically, two to six pieces of one liver (approx, 500 mg) or two pieces of muscle (approx, 180 mg) corresponding to ˜0.26 nM of Sirt1 in 20 μL of final lysate are used for a standard experiment to measure the activity of Sirt1 with five time points in triplicate for two given sets of experiment. The amount of Sirt1 in each preparation is again determined initially by Western-Blot analysis using different amounts of mouse liver lysates or muscle lysates with a given Sirt1 standard (purified Sirt1, bacterially expressed). 700 μL of assay buffer are added to the collected tissues and gently mixed. Then these tissues are homogenized on ice using a Polytron for 20 seconds at maximum speed. (Omni International GLH). The homogenized tissues are centrifuged for 5 minutes at 13.000 rpm and the supernatant (referred as “lysate”) is removed for further use in the activity assay.

Finally, lysates can also be prepared from cell lines, such as those derived from liver, muscle, fat etc. The following describes preparation of lysates from myoblast C2C12 cell line. Myoblast cells are grown to 80% confluence and harvested with TrypLE (Invitrogen), then washed twice with PBS buffer (Invitrogen) and stored at −80 degree Celsius prior to use. A C2C12 myoblast cell pellet ˜100 to 200 mg corresponding to ˜0.26 nM of Sirt1 in 20 μL of final lysate is used for a standard experiment to measure the activity of Sirt1 with five time points in triplicate for two given sets of experiment. The amount of Sirt1 in each preparation is determined initially by Western-Blot analysis using different amounts of cells with a given Sirt1 standard (purified Sirt1, bacterially expressed). 700 μL of assay buffer are added to the collected myoblast cells and gently mixed. Then these cells are sonicated on ice for 2 minutes with intervals (15 seconds sonication, 30 seconds pause) at a power output level of 1.5 with a small sonicator probe (Virsonic sonicator). The sonicated cells are centrifuged for 5 minutes at 3000 rpm and the supernatant (referred as “lysate”) is removed for further use in the activity assay. 20 uL of lysate are taken typically for one well of a 96 well plate with a final total reaction volume of 100 uL.

20 uL of lysate are taken typically for one well of a 96 well plate with a final total reaction volume of 100 uL. 1 μL of DMSO is added to each of the wells to give a final concentration of 1%. 29 uL of assay buffer are added to an initial volume of 50 uL. Stop buffer (10% trichloroacetic acid and 500 mM Nicotinamide) is added to the wells designated to zero time points. The activity assay is started by adding 50 uL of substrate buffer to each well. The substrate buffer consists of 20 μM Tamra peptide Ac-Glu-Glu-Lys(Biotin)-Gly-Gln-Ser-Thr-Ser-Ser-His-Ser-Lys(Ac)-Nle-Ser-Thr-Glu-Gly-Lys(5TMR)-Glu-Glu-NH2 (SEQ ID NO: 3) wherein K(Ac) is an acetylated lysine residue and Nle is a norleucine. The peptide is labeled with the fluorophore 5TMR (excitation 540 nm/emission 580 nm) at the C-terminus for use in the FP assay described above. The peptide substrate is prepared as a 1 mM stock in distilled water and stored in aliquots at −20° C.), 5 mM DTT, 0.05% BSA, 4 mM NAD⁺ and 10× reaction buffer. The reaction is performed at RT. For each time point the reaction will be stopped with stop buffer. After the final time point is collected the plates are sealed and analyzed by mass spectrometry.

As controls, specific Sirt1 and HDAC inhibitors are also included in the assay. Lysate volumes are adjusted accordingly to the amount needed for this inhibition assay. The following inhibitors are used with their respective final concentrations: 6-chloro-2,3,4,9-tetrahydro-1-H-carbazole-1-carboxamide (5 μM), TSA (1 μM) and nicotinamide (5 mM). 6-chloro-2,3,4,9-tetrahydro-1-H-carbazole-1-carboxamide and TSA are prepared in DMSO. Nicotinamide preparations are made in water. The final concentration of DMSO in each well is 1%. 1 μL of DMSO is added to wells containing Nicotinamide as inhibitor. The reactions are run in duplicate over a time period of 90 to 120 minutes with at least 5 time points taken.

Assay plates are transferred to BioTrove, Inc. (Woburn, Mass.) on dry ice for mass spectrometry analysis. Thawed reactions are analyzed using an Agilent 1100 HPLC with a microplate autosampler linked in series with a Sciex API-4000 mass spectrometer. Proprietary equipment (developed by BioTrove, Inc.) has been incorporated into this LC-MS system to allow for rapid sampling and rapid sample clean-up (4-5 sec per well). Both substrate and product are tracked in the MS and the area of the MS curve for both product and substrate are reported back in arbitrary units.

Using Microsoft Excel, plot product on the x axis and reaction time on the y axis of a xy scatter plot. The reaction is run at saturating substrate conditions with deliver a maximal turnover of substrate to product over a fixed time period, necessary for the detection of the activity of Sirt1. The final readout will be a number/slope describing product accumulation/time/ng of enzyme. Inhibition of the enzymatic activity of Sirt1 results in low product yields that enable the differentiation between HDAC's and Sirt1.

### Example 4

### Evaluation of Sirt1 Polymorphisms

Above, Applicants have demonstrated the link between a T373C or L107P Sirt1 polymorphic variant and diabetes. Using the methods below, one may characterize the relationship between T373N or L107X Sirt1 polymorphic variants and a number of other diseases and traits. The following example describes a protocol for establishing the interrelationship between T373N or L107X Sirt1 polymorphic variants and either environmental or physiological status or manipulation (diet, exercise, age, disease progression, etc.) or following pharmacological intervention (including Sirt1 modulators or other therapeutic interventions) including studies designed to study dose responsiveness and escalation, vehicle or placebo control versus treatment groups, dosing regimen, drug combination and synergy, etc. Cells, tissue or clinical samples (herein referred to as sample) can be from heart, kidney, brain, liver, bone marrow, colon, stomach, upper and lower intestine, breast, prostate, thyroid, gall bladder, lung, adrenals, muscle, fat, nerve fibers, pancreas, skin, eye, etc. Preferred samples include blood, white blood cells, liver, muscle, fat and other tissues that are the target of Sirt1 pharmacological intervention. The cells or tissues can be pretreated with Sirt1 modulators or other pharmacological agents either in vivo or following isolation.

The genetic analysis of haplotypes, SNPs or alleles of the Sirt1 gene as described herein could be done on the samples collected above. It is of course understood that in general the genetic analysis need not be done on the same sample used for subsequent biochemical analysis. Any sample, tissue or biopsy obtained from the given patient should be sufficient to determine the genetic haplotype of the Sirt1 gene as well as genetic analysis of any other gene. The haplotype may be schematically represented as +/+, +/− or −/− for the Sirt1 allele of interest.

The sample can then be subjected to a number of other biochemical and/or biological studies. These include quantitative measurement of mRNA or protein by methods known in the art and described herein. Of particular interest would be the measurement of Sirt1 mRNA or protein. Other gene products of interest include the PGC-1α mRNA and protein and genes related to OXPHOS (Lin et al., 2002, J. Biol. Chem. 277, 1645-1648); the estrogen related receptor alpha (ERRα) and nuclear respiratory factor1 (NRF-1) mRNA and protein (Mootha et al., 2004, Proc Natl Acad Sci USA 101, 6570-6575; Patti et al., 2003, Proc Natl Acad Sci USA 100, 8466-8471); Mitochondrial transcription factor A (Tfam), a nuclear encoded mitochondrial transcription factor that is indispensable for the expression of key mitochondrial-encoded genes (Larsson et al., 1998, Nat. Genet. 18, 231-236) and a target of NRF-1; an array of additional downstream targets of PGC-1α (Lin et al., 2005, Cell Metab 1, 361-370), including genes involved in fatty acid oxidation (medium chain acyl-CoA dehydrogenase, MCAD), uncoupling and protection against ROS (uncoupling protein 3, UCP-3), and fiber type markers (myoglobin and troponin 1).

In addition to the measurement of protein and mRNA of specific gene products as described above, the measurement of endogenous activity can be measured. This includes the determination of endogenous sirtuin activity in various clinical samples with or without physiological manipulation or pharmacological intervention. Of particular interest is the measurement of endogenous sirtuin activity as described in Example 2 above. Other activities can also be measured, including citrate synthase as described above in Example 3, ATP synthase, or where possible, any of the other gene products described above in this example. Finally, other mRNA, protein and/or activities that could be measured include those associated with mitochondrial biogenesis and disease progression or pathogenesis as described elsewhere in this specification. This includes ATP levels, mitochondrial number and size, mitochondrial DNA, oxidative phosphorylation markers, reactive oxygen species, etc. Specific mRNA and protein levels can be measured for the following: Sirt1, PGC-1alpha, mtTFA (TFAM), UQCRB, Citrate synthase, Foxol, PPARgamma2, PPARdelta, LXRalpha, ABCA1, aP2, Fatty acid synthase, Adiponectin (13 genes), PGC-1beta, PPARgamma1, MIF (macrophage migration inhibition factor), MMP-9, TNFalpha, IL-1alpha, IL-1beta, IL-12alpha, IL-18, IL-18BP (IL-18 binding protein), COX2 (cyclooxygenase-2), Lipoprotein lipase (LPL), resistin, IL-8, IL8Receptor, MCPJ, MCP1-Receptor, MIP1alpha, MIP2alpha, MIP2beta, MMP-10, MIP1, VCAM, IL-6, TLR4, TLR2, ANGP1.

The correlation can then be made between Sirt1 haplotype (in combination with genetic analysis of other genes of interest) with mRNA, protein and activity of Sirt1 or any of the other gene products described above. This analysis can then be extended to preclinical or clinical outcome analysis, especially when looking at pharmacological intervention, herein referred to as pharmacogenetics or pharmacogenomics. This includes prevention and/or intervention in diseases or disorders including reversal of disease or slowing the rate of progression, attenuation of disease markers, or holding of disease status or limiting disease progression. Specific diseases or disorders include those related to aging or stress, diabetes, obesity, neurodegenerative diseases, diseases or disorders associated with mitochondrial dysfunction, chemotherapeutic induced neuropathy, neuropathy associated with an ischemic event, ocular diseases and/or disorders, cardiovascular disease, blood clotting disorders, inflammation, oncology, asthma, COPD, rheumatoid arthritis, irritable bowel syndrome, psoriasis, and/or flushing, etc. Efficacy readouts for metabolic, diabetes or obesity related indications include glycosylated HbA1C, fasting or post prandial glucose levels, glucose tolerance or insulin sensitivity, plasma insulin levels, etc. for metabolic indications. Other readouts include core body temperature, exercise endurance, energy expenditure, reactive oxygen species (ROS) levels, and other measurements of mitochondrial function or biogenesis as described herein. Neurological indications and clinical readouts include those known in the art and include such diseases as, for example, AD (Alzheimer's Disease), multiple sclerosis (MS), ADPD (Alzheimer's Disease and Parkinsons's Disease), HD (Huntington's Disease), PD (Parkinson's Disease), Friedreich's ataxia and other ataxias, amyotrophic lateral sclerosis (ALS) and other motor neuron diseases, optic neuritis, glaucoma and other related eye diseases, MELAS and LHON. Based on haplotype analysis, biochemical and clinical parameters, clinical intervention can be assessed based on dose responsiveness and escalation, vehicle or placebo control versus treatment groups, dosing regimen, drug combination and synergy, etc.

### EQUIVALENTS

The present invention provides among other things predictive and diagnostic methods using polymorphic variants of Sirt1. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

### INCORPORATION BY REFERENCE

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) (www.tigr.org) and/or the National Center for Biotechnology Information (NCBI) (www.ncbi.nlm.nih.gov).

1. An isolated Sirt1 nucleic acid having a T373N substitution comprising: SEQ ID NO: 1 or a nucleotide sequence having 97% identity to SEQ ID NO: 1, wherein the nucleotide at position 373 is adenine, guanine, or cytosine.

2. The isolated Sirt1 nucleic acid of claim 1, wherein N is cytosine.

3. The isolated Sirt1 nucleic acid of claim 1, comprising the amino acid sequence of SEQ ID NO: 1, wherein N is cytosine.

4. The isolated Sirt1 nucleic acid of claim 1, which is operably linked to a nucleotide tag sequence.

5. The isolated Sirt1 nucleic acid of claim 1, which is operably linked to a promoter sequence.

6. A vector comprising the isolated Sirt1 nucleic acid of claim 1.

7. An isolated oligonucleotide comprising 20 to 100 consecutive nucleotides of SEQ ID NO: 1 or the complement thereof, wherein nucleotide 373 is included in said oligonucleotide.

8. The isolated oligonucleotide of claim 7, which is attached to a solid substrate.

9. The isolated oligonucleotide of claim 7, wherein said oligonucleotide further comprises a detectable label.

10. A microarray comprising a plurality of oligonucleotides attached to a solid substrate, wherein at least one oligonucleotide is the oligonucleotide of claim 7.

11. An isolated Sirt1 polypeptide having a L107X substitution comprising: SEQ ID NO: 2 or an amino acid sequence having 99% identity to SEQ ID NO: 2, wherein the amino acid at position 107 is any amino acid other than leucine.

12. The isolated Sirt1 polypeptide of claim 11, wherein X is proline.

13. The isolated Sirt1 polypeptide of claim 11, comprising the amino acid sequence of SEQ ID NO: 2, wherein X is proline.

14. The isolated Sirt1 polypeptide of claim 11, which is operably linked to a polypeptide tag sequence.

15. An isolated nucleic acid encoding the Sirt1 polypeptide of claim 11.

16. A host cell comprising the nucleic acid of claim 1.

17. A transgenic non-human mammal comprising the nucleic acid of claim 1.

18. A method for evaluating a subject's risk of developing a sirtuin-mediated disease or disorder, comprising determining the identity of nucleotide 373 or amino acid 107 of Sirt1 in a biological sample from said subject, wherein a nucleotide other than thymine at position 373 or an amino acid other than leucine at position 107 is indicative of a subject having an altered risk for developing a sirtuin-mediated disease or disorder.

19. The method of claim 18, wherein the identity of nucleotide 373 is determined by nucleic acid sequencing, primer extension, restriction enzyme cleavage pattern, or by use of a nucleic acid probe that hybridizes to the nucleic acid sequence.


SEQUENCE LISTING (46 sequences)

SEQ ID NO: 1 - DNA, 4086 bp, Homo sapien
  misc_feature 373: n = A,T,C or G
gtcgagcgggagcagaggaggcgagggaggagggccagagaggcagttggaagatggcggacgaggcggccctcgcccttcagcccggcggctccccctcggcggcgggggccgacagggaggccgcgtcgtcccccgccggggagccgctccgcaagaggccgcggagagatggtcccggcctcgagcggagcccgggcgagcccggtggggcggccccagagcgtgaggtgccggcggcggccaggggctgcccgggtgcggcggcggcggcgctgtggcgggaggcggaggcagaggcggcggcggcaggcggggagcaagaggcccaggcgactgcggcggctggggaaggagacaatgggccgggccngcagggcccatctcgggagccaccgctggccgacaacttgtacgacgaagacgacgacgacgagggcgaggaggaggaagaggcggcggcggcggcgattgggtaccgagataaccttctgttcggtgatgaaattatcactaatggttttcattcctgtgaaagtgatgaggaggatagagcctcacatgcaagctctagtgactggactccaaggccacggataggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaacacaaaaagaattggcttatttgtcagagttgccacccacacctcttcatgtttcagaagactcaagttcaccagaaagaacttcaccaccagattcttcagtgattgtcacacttttagaccaagcagctaagagtaatgatgatttagatgtgtctgaatcaaaaggttgtatggaagaaaaaccacaggaagtacaaacttctaggaatgttgaaagtattgctgaacagatggaaaatccggatttgaagaatgttggttctagtactggggagaaaaatgaaagaacttcagtggctggaacagtgagaaaatgctggcctaatagagtggcaaaggagcagattagtaggcggcttgatggtaatcagtatctgtttttgccaccaaatcgttacattttccatggcgctgaggtatattcagactctgaagatgacgtcttatcctctagttcttgtggcagtaacagtgatagtgggacatgccagagtccaagtttagaagaacccatggaggatgaaagtgaaattgaagaattctacaatggcttagaagatgagcctgatgttccagagagagctggaggagctggatttgggactgatggagatgatcaagaggcaattaatgaagctatatctgtgaaacaggaagtaacagacatgaactatccatcaaacaaatcatagtgtaataattgtgcaggtacaggaattgttccaccagcattaggaactttagcatgtcaaaatgaatgtttacttgtgaactcgatagagcaaggaaaccagaaaggtgtaatatttataggttggtaaaatagattgtttttcatggataatttttaacttcattatttctgtacttgtacaaactcaacactaacttttttttttttaaaaaaaaaaaggtactaagtatcttcaatcagctgttgggtcaagactaactttcttttaaaggttcatttgtatgataaattcatatgtgtatatataattttttttgttttgtctagtgagtttcaacatttttaaagttttcaaaaagccatcggaatgttaaattaatgtaaagggacagctaatctagaccaaagaatggtattttcacttttctttgtaacattgaatggtttgaagtactcaaaatctgttacgctaaacttttgattctttaacacaattatttttaaacactggcattttccaaaactgtggcagctaactttttaaaatctcaaatgacatgcagtgtgagtagaaggaagtcaacaatatgtggggagagcactcggttgtctttacttttaaaagtaatacttggtgctaagaatttcaggattattgtatttacgttcaaatgaagatggcttttgtacttcctgtggacatgtagtaatgtctatattggctcataaaactaacctgaaaaacaaataaatgctttggaaatgtttcagttgctttagaaacattagtgcctgcctggatccccttagttttgaaatatttgccattgttgtttaaatacctatcactgtggtagagcttgcattgatcttttccacaagtattaaactgccaaaatgtgaatatgcaaagcctttctgaatctataataatggtacttctactggggagagtgtaatattttggactgctgttttccattaatgaggagagcaacaggcccctgattatacagttccaaagtaataagatgttaattgtaattcagccagaaagtacatgtctcccattgggaggatttggtgttaaataccaaactgctagccctagtattatggagatgaacatgatgatgtaacttgtaatagcagaatagttaatgaatgaaactagttcttataatttatctttatttaaaagcttagcctgccttaaaactagagatcaactttctcagctgcaaaagcttctagtctttcaagaagttcatactttatgaaattgcacagtaagcatttatttttcagaccatttttgaacatcactcctaaattaataaagtattcctctgttgctttagtatttattacaataaaaagggtttgaaatatagctgttctttatgcataaaacacccagctaggaccattactgccagagaaaaaaatcgtattgaatggccatttccctacttataagatgtctcaatctgaatttatttggctacactaaagaatgcagtatatttagttttccatttgcatgatgtttgtgtgctatagatgatattttaaattgaaaagtttgttttaaattatttttacagtgaagactgttttcagctctttttatattgtacatagtcttttatgtaatttactggcatatgttttgtagactgtttaatgactggatatcttccttcaacttttgaaatacaaaaccagtgttttttacttgtacactgttttaaagtctattaaaattgtcatttgacttttttctg

SEQ ID NO: 2 - protein, 747 aa, Homo sapien
  VARIANT 107: Xaa = Any Amino Acid
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGXQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRTQKELAYLSELPPTPLHVSEDSSSPERTSPPDSSVIVTLLDQAAKSNDDLDVSESKGCMEEKPQEVQTSRNVESIAEQMENPDLKNVGSSTGEKNERTSVAGTVRKCWPNRVAKEQISRRLDGNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 3 - protein, 20 aa, Artificial Sequence
  Synthetic peptide substrate for the SIRT1 enzyme
EEKGQSTSSHSKLSTEGKEE

SEQ ID NO: 4 - DNA, 23 bp, Artificial Sequence
  PCR primer
gcgggagcagaggaggcgaggga

SEQ ID NO: 5 - DNA, 23 bp, Artificial Sequence
  PCR primer
gggccggggtggggaggggaacg

SEQ ID NO: 6 - DNA, 23 bp, Artificial Sequence
  PCR primer
ggcagttggaagatggcggacga

SEQ ID NO: 7 - DNA, 23 bp, Artificial Sequence
  PCR primer
ggagcaagaggcccaggcgactg

SEQ ID NO: 8 - DNA, 23 bp, Artificial Sequence
  PCR primer
gtacccaatcgccgccgccgccg

SEQ ID NO: 9 - DNA, 23 bp, Artificial Sequence
  PCR primer
acacacgccgtgtaaatgttaaa

SEQ ID NO: 10 - DNA, 23 bp, Artificial Sequence
  PCR primer
cggatcacctgaggtcgggagtt

SEQ ID NO: 11 - DNA, 23 bp, Artificial Sequence
  PCR primer
gccgtgtaaatgttaaatgctgt

SEQ ID NO: 12 - DNA, 23 bp, Artificial Sequence
  PCR primer
actccagcctccgcaacaagagc

SEQ ID NO: 13 - DNA, 23 bp, Artificial Sequence
  PCR primer
gtcttcccaactcttcattagat

SEQ ID NO: 14 - DNA, 23 bp, Artificial Sequence
  PCR primer
agttcccaaacatccattatcat

SEQ ID NO: 15 - DNA, 23 bp, Artificial Sequence
  PCR primer
cttaaagatttattgccggaaac

SEQ ID NO: 16 - DNA, 22 bp, Artificial Sequence
  PCR primer
gtttccggcaataaatcttaag

SEQ ID NO: 17 - DNA, 23 bp, Artificial Sequence
  PCR primer
ctgaaataagggtagggttgtat

SEQ ID NO: 18 - DNA, 23 bp, Artificial Sequence
  PCR primer
atcttactccgccacagtagcag

SEQ ID NO: 19 - DNA, 23 bp, Artificial Sequence
  PCR primer
gatggtatttatgctcgccttgc

SEQ ID NO: 20 - DNA, 22 bp, Artificial Sequence
  PCR primer
ttactccgccacagtgcagcat

SEQ ID NO: 21 - DNA, 23 bp, Artificial Sequence
  PCR primer
agtctacagcaaggcgagcataa

SEQ ID NO: 22 - DNA, 23 bp, Artificial Sequence
  PCR primer
agagatgtttatgggccgacttt

SEQ ID NO: 23 - DNA, 23 bp, Artificial Sequence
  PCR primer
ttaactgtcaaggccaatacttc

SEQ ID NO: 24 - DNA, 23 bp, Artificial Sequence
  PCR primer
cctttggattcccgcaacctgtt

SEQ ID NO: 25 - DNA, 23 bp, Artificial Sequence
  PCR primer
aacaggttgcgggaatccaaagg

SEQ ID NO: 26 - DNA, 22 bp, Artificial Sequence
  PCR primer
caaaacaaaaatccttaaaccc

SEQ ID NO: 27 - DNA, 23 bp, Artificial Sequence
  PCR primer
acattttatgttcggcttagata

SEQ ID NO: 28 - DNA, 23 bp, Artificial Sequence
  PCR primer
aaagttgactgtgaagctgtacg

SEQ ID NO: 29 - DNA, 23 bp, Artificial Sequence
  PCR primer
cgtacagcttcacagtcaacttt

SEQ ID NO: 30 - DNA, 23 bp, Artificial Sequence
  PCR primer
gactatttctaacttgggcttac

SEQ ID NO: 31 - DNA, 23 bp, Artificial Sequence
  PCR primer
gtctattatacagacccacaacc

SEQ ID NO: 32 - DNA, 23 bp, Artificial Sequence
  PCR primer
gggcttactctttgcttctctac

SEQ ID NO: 33 - DNA, 23 bp, Artificial Sequence
  PCR primer
tatagaaatgttctccaaaaacc

SEQ ID NO: 34 - DNA, 23 bp, Artificial Sequence
  PCR primer
cccttgttggatttttgcataat

SEQ ID NO: 35 - DNA, 23 bp, Artificial Sequence
  PCR primer
aatcaactacataaagctaccct

SEQ ID NO: 36 - DNA, 23 bp, Artificial Sequence
  PCR primer
ttatttttcaccctatttaggtt

SEQ ID NO: 37 - DNA, 23 bp, Artificial Sequence
  PCR primer
cgattaacctgccgaaatagcta

SEQ ID NO: 38 - DNA, 23 bp, Artificial Sequence
  PCR primer
gaaccaacattcttcaaatccgg

SEQ ID NO: 39 - DNA, 23 bp, Artificial Sequence
  PCR primer
aaggcagagctggaacccacact

SEQ ID NO: 40 - DNA, 23 bp, Artificial Sequence
  PCR primer
ccaacagctgattgaagatactt

SEQ ID NO: 41 - DNA, 23 bp, Artificial Sequence
  PCR primer
tgccagagtccaagtttagaaga

SEQ ID NO: 42 - DNA, 23 bp, Artificial Sequence
  PCR primer
tcttctaaacttggactctggca

SEQ ID NO: 43 - DNA, 430 bp, Homo sapien
  CDS (1)...(430)
atggcggacgaggcggccctcgcccttcagcccggcggctccccctcggcggcgggggccgacagggaggccgcgtcgtcccccgccggggagccgctccgcaagaggccgcggagagatggtcccggcctcgagcggagcccgggcgagcccggtggggcggccccagagcgtgaggtgccggcggcggccaggggctgcccgggtgcggcggcggcggcgctgtggcgggaggcggaggcagaggcggcggcggcaggcggggagcaagaggcccaggcgactgcggcggctggggaaggagacaatgggccgggcccgcagggcccatctcgggagccaccgctggccgacaacttgtacgacgaagacgacgacgacgagggcgaggaggaggaagaggcggcggcggcggcgattgggtaccgag

SEQ ID NO: 44 - protein, 143 aa, Homo sapien
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGPQGPSREPPLADNLYDEDDDDEGEEEEEAAAAAIGYR

SEQ ID NO: 45 - DNA, 430 bp, Homo sapien
  CDS (1)...(430)
atggcggacgaggcggccctcgcccttcagcccggcggctccccctcggcggcgggggccgacagggaggccgcgtcgtcccccgccggggagccgctccgcaagaggccgcggagagatggtcccggcctcgagcggagcccgggcgagcccggtggggcggccccagagcgtgaggtgccggcggcggccaggggctgcccgggtgcggcggcggcggcgctgtggcgggaggcggaggcagaggcggcggcggcaggcggggagcaagaggcccaggcgactgcggcggctggggaaggagacaatgggccgggcctgcagggcccatctcgggagccaccgctggccgacaacttgtacgacgaagacgacgacgacgagggcgaggaggaggaagaggcggcggcggcggcgattgggtaccgag

SEQ ID NO: 46 - protein, 143 aa, Homo sapien
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYR